(19) United States
(12) Patent Application Publication
(10) Pub. No.: US 2023/0015616 A1
(43) Pub. Date: Jan. 19, 2023

(54) CORONAVIRUS VACCINES AND USES THEREOF

(71) Applicant: ARGORNA PHARMACEUTICALS LTD, Guangzhou, Guandong (CN)

(72) Inventors: Bill Biliang ZHANG, Guangzhou, Guangdong (CN); Man LI, Guangzhou, Guangdong (CN); Lin MA, Guangzhou, Guangdong (CN); Jian WEN, Guangzhou, Guangdong (CN); Hong ZHANG, Guangzhou, Guangdong (CN); Huiling ZHONG, Guangzhou, Guangdong (CN)

(21) Appl. No.: 17/437,266

(22) PCT Filed: Nov. 30, 2020
(86) PCT No.: PCT/CN2020/132609

§ 371 Date: Sep. 8, 2021

(51) Int. Cl. A61K 39/215; A61P 31/14; C07K 14/165

(52) U.S. Cl. CPC A61K 39/215; A61P 31/14; C07K 14/165; A61K 2039/53

(57) ABSTRACT

This disclosure relates to coronavirus vaccines and uses thereof. In one aspect, the disclosure provides a nucleic acid vaccine, comprising a sequence encoding a spike protein or fragment thereof derived from a coronavirus.

### TECHNICAL FIELD

This disclosure relates to coronavirus vaccines and uses thereof.

### BACKGROUND

Coronaviruses are enveloped, positive-sense single-strand RNA viruses with mammalian and avian hosts. Previous coronaviruses known to infect humans include 229E, NL63, OC43, HKU1, SARS-CoV, and MERS-CoV, which cause a range of mild seasonal illnesses to severe diseases outbreaks. Notably, the past outbreaks of severe acute respiratory syndrome (SARS) (2003) and Middle East respiratory syndrome (MERS) (2012) were caused by the coronaviruses SARS-CoV and MERS-CoV, respectively (See Human Coronavirus Types: Centers for Disease Control and Prevention). SARS-CoV-2, which emerged in December 2019, is the seventh known coronavirus to infect humans, and the third coronavirus to cross species barriers and cause severe respiratory infections in humans in less than two decades after SARS and MERS. It causes the coronavirus disease 2019 (COVID-19) (See Okba N M A, Muller M A, Li W, et al. Severe Acute Respiratory Syndrome Coronavirus 2-Specific Antibody Responses in Coronavirus Disease 2019 Patients. Emerg Infect Dis. 2020 Apr. 8; 26(7)) that is more contagious than SARS-CoV and MERS-CoV.

The high rate of infection and worldwide impact caused by the disease led the World Health Organization to declare COVID-19 a pandemic. As of October 2020, 39 million cases have been reported across 189 countries and territories, but the WHO estimates that around 800 million people in total may have been infected. The disease has killed 1.1 million people. There is an urgent need for effective vaccines against coronavirus.

### SUMMARY

This disclosure relates to coronavirus vaccines and the uses thereof.

In one aspect, the disclosure provides a nucleic acid encoding a modified spike protein or fragment thereof derived from a coronavirus. In some embodiments, the modified spike protein or fragment thereof is locked in a pre-fusion state. In some embodiments, the coronavirus is SARS-CoV-2.

In some embodiments, the modified spike protein or fragment thereof meets either one or both of the conditions: (a) the amino acid that corresponds to K986 of SEQ ID NO: 1 is not Lys; and (b) the amino acid that corresponds to V987 of SEQ ID NO: 1 is not Val.

In some embodiments, the amino acid in the modified spike protein or fragment thereof that corresponds to K986 of SEQ ID NO: 1 is proline. In some embodiments, the amino acids in the modified spike protein or fragment thereof that corresponds to V987 of SEQ ID NO: 1 is proline. In some embodiments, both of the amino acids in the modified spike protein or fragment thereof that correspond to K986 and V987 of SEQ ID NO: 1 are proline.

In some embodiments, the modified spike protein or fragment thereof comprises from N-terminus to C-terminus: an N-terminal domain (NTD), a receptor binding domain (RBD), and a heptad repeat region. In some embodiments, the heptad repeat region comprises a first heptad repeat (HR1) and a second heptad repeat (HR2).

In some embodiments, the N-terminal domain comprises a sequence that is at least 80% identical to amino acids 14-305 of SEQ ID NO: 1 In some embodiments, the receptor binding domain comprises a sequence that is at least 80%, 90%, 95% or 100% identical to amino acids 319-541 of SEQ ID NO: 1. In some embodiments, the heptad repeat region comprises a sequence that is at least 80%, 90%, 95% or 100% identical to amino acids 912-1213 of SEQ ID NO: 1. In some embodiments, the modified spike protein or fragment thereof further comprises a transmembrane domain (TM). In some embodiments, the transmembrane domain comprises a sequence that is at least 80% identical to amino acids 1214-1237 of SEQ ID NO: 1.

In some embodiments, the modified spike protein or fragment thereof comprises or consists of an extracellular domain. In some embodiments, the modified spike protein or fragment thereof does not have a membrane fusion peptide domain (e.g., the amino acids that correspond to positions 788-806 of SEQ ID NO: 1).

In some embodiments, the modified spike protein or fragment thereof is resistant to protease cleavage. In some embodiments, the amino acids in the modified spike protein or fragment thereof that correspond to positions 682-685 of SEQ ID NO: 1 are GGSG. In some embodiments, the amino acids in the modified spike protein or fragment thereof that correspond to positions 814 and 815 of SEQ ID NO: 1 are AN.

In some embodiments, the modified spike protein or fragment thereof further comprises a signal peptide. In some embodiments, the signal peptide comprises a sequence that is at least 80% identical to amino acids 1-13 of SEQ ID NO: 1. In some embodiments, the signal peptide is an immunoglobulin heavy chain variable region (IGVH) signal peptide. In some embodiments, the immunoglobulin heavy chain variable region (IGVH) signal peptide comprises a sequence that is at least 80%, 90%, 95% or 100% identical to SEQ ID NO: 39.

In some embodiments, the modified spike protein or fragment thereof further comprises a T4 phage fibritin trimer motif. In some embodiments, the T4 phage fibritin trimer motif comprises a sequence that is at least 80%, 90%, 95% or 100% identical to SEQ ID NO: 40.

In some embodiments, the modified spike protein or fragment thereof further comprises a linker peptide sequence (e.g., SAIG (SEQ ID NO: 54)).

In some embodiments, the nucleic acid comprises a 5′-UTR. In some embodiments, the 5′-UTR is a Kozak sequence (SEQ ID NO. 42) or the 5′-UTR of the following genes: HBB (Hemoglobin Subunit Beta), Hsp70, DNAH2 (Dynein Axonemal Heavy Chain 2), or HSD17B4 (Hydroxysteroid 17-Beta Dehydrogenase 4).

In some embodiments, the modified spike protein or fragment thereof forms a trimer.

In some embodiments, the modified spike protein or fragment thereof comprises a sequence that is at least 80%, 90%, 95% or 100% identical to any one of SEQ ID NOs: 3-38, with or without amino acids 1-13 of SEQ ID NOs: 3-38.

In some embodiments, the modified spike protein or fragment thereof comprises a sequence that is at least 80%, 90%, 95% or 100% identical to amino acids 14-1213 of SEQ ID NO: 29.

In some embodiments, the modified spike protein or fragment thereof comprises a sequence that is at least 80%, 90%, 95% or 100% identical to amino acids 14-1194 of SEQ ID NO: 32.

In some embodiments, the modified spike protein or fragment thereof comprises a sequence that is at least 80%, 90%, 95% or 100% identical to amino acids 14-1237 of SEQ ID NO: 35.

In some embodiments, the modified spike protein or fragment thereof comprises a sequence that is at least 80%, 90%, 95% or 100% identical to amino acids 14-1218 of SEQ ID NO: 38.

In some embodiments, the modified spike protein or fragment thereof further comprises is an immunoglobulin heavy chain variable region (IGVH) signal peptide.

In some embodiments, the modified spike protein or fragment thereof comprises a sequence that is at least 80%, 90%, 95% or 100% identical to SEQ ID NO: 56. In some embodiments, the modified spike protein or fragment thereof comprises a sequence that is at least 80%, 90%, 95% or 100% identical to SEQ ID NO: 57. In some embodiments, the modified spike protein or fragment thereof comprises a sequence that is at least 80%, 90%, 95% or 100% identical to SEQ ID NO: 58. In some embodiments, the modified spike protein or fragment thereof comprises a sequence that is at least 80%, 90%, 95% or 100% identical to SEQ ID NO: 59.

In one aspect, the disclosure provides a nucleic acid encoding a modified spike protein or fragment thereof derived from a coronavirus. In some embodiments, the nucleic acid comprises a sequence that is at least 80%, 90%, 95% or 100% identical to any one of SEQ ID NOs: 2 and 44-52.

In some embodiments, the nucleic acid comprises a sequence that is at least 80%, 90%, 95% or 100% identical to SEQ ID NO: 49. In some embodiments, the nucleic acid comprises a sequence that is at least 80%, 90%, 95% or 100% identical to SEQ ID NO: 50. In some embodiments, the nucleic acid comprises a sequence that is at least 80%, 90%, 95% or 100% identical to SEQ ID NO: 51. In some embodiments, the nucleic acid comprises a sequence that is at least 80%, 90%, 95% or 100% identical to SEQ ID NO: 52.

In some embodiments, the nucleic acid comprises from 5′ end to 3′ end the following elements: a) 5′-UTR; b) Kozak sequence; c) an open reading frame encoding the modified spike protein or fragment thereof; d) 3′-UTR; and e) a polyA tail.

In some embodiments, the nucleic acid is a DNA molecule. In some embodiments, the nucleic acid is an RNA molecule. In some embodiments, the nucleic acid is a RNA molecule with one or more modified nucleosides.

In some embodiments, the one or more modified nucleosides are selected from pseudo-UTP, 5-Me-CTP, rUTP, 1-N-Me-pseudo-UTP, or a combination thereof.

In one aspect, the disclosure provides an expression vector comprising the nucleic acid of as described herein and a promoter. In some embodiments, the promoter is operably linked to the nucleic acid.

In one aspect, the disclosure provides a modified coronavirus spike protein or fragment thereof, comprising from N-terminus to C-terminus: an N-terminal domain (NTD), a receptor binding domain (RBD), and a heptad repeat region. In some embodiments, the heptad repeat region comprises a first heptad repeat (HR1) and a second heptad repeat (HR2). In some embodiments, the modified spike protein or fragment thereof is locked in a pre-fusion state (e.g., a closed pre-fusion state).

In some embodiments, the modified spike protein or fragment thereof meets either one or both of the conditions: (a) the amino acid that corresponds to K986 of SEQ ID NO: 1 is not Lys (e.g., is Asp or Glu); and (b) the amino acid that corresponds to V987 of SEQ ID NO: 1 is not V (e.g., is Phe, Try, or Trp).

In some embodiments, either one or both of the amino acids corresponding to K986 and V987 of SEQ ID NO: 1 are proline.

In some embodiments, the N-terminal domain comprises a sequence that is at least 80%, 90%, 95% or 100% identical to amino acids 14-305 of SEQ ID NO: 1. In some embodiments, the receptor binding domain comprises a sequence that is at least 80%, 90%, 95% or 100% identical to amino acids 319-541 of SEQ ID NO: 1. In some embodiments, the heptad repeat region comprises a sequence that is at least 80%, 90%, 95% or 100% identical to amino acids 912-1213 of SEQ ID NO: 1.

In some embodiments, the modified coronavirus spike protein or fragment thereof further comprises a transmembrane domain (TM). In some embodiments, the transmembrane domain comprises a sequence that is at least 80%, 90%, 95% or 100% identical to amino acids 1214-1237 of SEQ ID NO: 1. In some embodiments, the modified coronavirus spike protein or fragment thereof does not have a membrane fusion peptide domain (e.g., the amino acids that correspond to positions 788-806 of SEQ ID NO: 1).

In some embodiments, the modified spike protein or fragment thereof is resistant to protease cleavage (e.g., Furin-like protease cleavage). In some embodiments, the amino acids that correspond to positions 682-685 of SEQ ID NO: 1 are GGSG (SEQ ID NO: 53). In some embodiments, the amino acids that correspond to positions 814 and 815 of SEQ ID NO: 1 are AN.

In some embodiments, the modified coronavirus spike protein or fragment thereof further comprises a signal peptide. In some embodiments, the signal peptide comprises a sequence that is at least 80%, 90%, 95% or 100% identical to amino acids 1-13 of SEQ ID NO: 1. In some embodiments, the signal peptide is an immunoglobulin heavy chain variable region (IGVH) signal peptide (e.g., a sequence that is at least 80%, 90%, 95% or 100% identical to SEQ ID NO: 39).

In some embodiments, the modified coronavirus spike protein or fragment thereof further comprises a linker peptide sequence and a T4 phage fibritin trimer motif. In some embodiments, the linker peptide sequence is SAIG (SEQ ID NO: 54). In some embodiments, the T4 phage fibritin trimer motif comprises a sequence that is at least 80%, 90%, 95% or 100% identical to SEQ ID NO: 40.

In one aspect, the disclosure provides a protein complex comprising three coronavirus spike proteins or fragments thereof. In some embodiments, each of the three coronavirus spike proteins or fragments comprises modified coronavirus spike protein or fragment thereof as described herein.

In one aspect, the disclosure provides a vaccine comprising the nucleic acids as described herein, the modified coronavirus spike protein or fragment thereof as described herein, or the protein complex as described herein.

In one aspect, the disclosure provides a pharmaceutical composition comprising the nucleic acids as described herein, the modified coronavirus spike protein or fragment thereof as described herein, or the protein complex as described herein; and a pharmaceutical carrier.

In one aspect, the disclosure provides a lipid nanoparticle comprising the nucleic acids as described herein, the modified coronavirus spike protein or fragment thereof as described herein, or the protein complex as described herein.

In one aspect, the disclosure provides a method of inducing an immune response to coronavirus in a subject. In some embodiments, the method comprises administering to a subject in need thereof the nucleic acids as described herein, the modified coronavirus spike protein or fragment thereof as described herein, the protein complex as described herein, the vaccine as described herein, the pharmaceutical composition as described herein, or the liposome nanoparticle as described herein. In some embodiments, the subject develops an immune response to coronavirus within 14 days of the administration.

In some embodiments, at least 2 doses are administered to the subject. In some embodiments, the second dose is administered at least 10 days (e.g., 14 days) after the first dose is administered to the subject.

In some embodiments, the subject maintains the immune response to coronavirus for at least 3 months. In some embodiments, the subject develops a neutralizing antibody against coronavirus in response.

In one aspect, the disclosure provides a method of increasing an immune response to coronavirus in a subject or treating a subject having coronavirus, the method comprising: administering to the subject in need thereof the nucleic acids as described herein, the modified coronavirus spike protein or fragment thereof as described herein, the protein complex as described herein, the vaccine as described herein, the pharmaceutical composition as described herein, or the liposome nanoparticle as described herein.

In some embodiments, the subject is in coronavirus incubation period.

In one aspect, the disclosure provides a method of making a nucleic acid vaccine comprising: synthesizing the nucleic acid as described herein.

In some embodiments, the nucleic acid has been optimized for expression and/or translation efficiency.

In some embodiments, the nucleic acid is synthesized in the presence of pseudo-UTP. In some embodiments, the nucleic acid is synthesized in the presence of 5-Me-CTP, and 1-N-Me-Pseudo-UTP. In some embodiments, the nucleic acid is synthesized in a solution in the presence of rUTP and 1-N-Me-Pseudo-UTP. In some embodiments, the ratio of rUTP to 1-N-Me-Pseudo-UTP is between 1.5:1 and 1:1.5 (e.g., roughly about 1:1). In some embodiments, the nucleic acid is synthesized in the presence of 1-N-Me-Pseudo-UTP.

In some embodiments, the method further comprises packaging the nucleic acid in a liposomal nanoparticle.

In one aspect, the disclosure provides a cell comprising the nucleic acids as described herein. In one aspect, the disclosure provides a cell expresses the modified coronavirus spike protein or fragment thereof as described herein.

In one aspect, the disclosure provides a method of making an antibody the specifically binds to S protein, the method comprising immunizing an animal with the nucleic acids as described herein, the modified coronavirus spike protein or fragment thereof as described herein, the protein complex as described herein, the vaccine as described herein, the pharmaceutical composition as described herein, and/or the liposome nanoparticle as described herein.

In some embodiments, the animal is a non-human mammal (e.g., a mouse). In some embodiments, the method further comprises humanizing the antibody.

The present disclosure is related to an mRNA vaccine that induces production of antibodies against coronavirus S protein. In some embodiments, the mRNA encodes a SARS-CoV-2 S protein in a pre-fusion stable form. In some embodiments, the S protein forms a trimer.

In one aspect, the disclosure is related to a spike protein (S protein) or polypeptide of SARS-CoV-2. In some embodiments, the modified S protein comprises a sequence that is at least or about 70%, 75%, 80%, 85%, 90%, 95%, 96%, 97%, 98%, 99%, or 100% identical to the ectodomain sequence of a wild-type S protein (e.g., amino acids 1-1213 of SEQ ID NO: 1). In some embodiments, the modified S protein comprises a sequence that is at least or about 70%, 75%, 80%, 85%, 90%, 95%, 96%, 97%, 98%, 99%, or 100% identical to the ectodomain and transmembrane domain sequence of a wild-type S protein (e.g., amino acids 1-1237). In some embodiments, the modified S protein is a soluble protein. In some embodiments, the modified S protein is a membrane-anchored protein.

In some embodiments, the modified S protein does not comprise the transmembrane and cytoplasmic region of a wild-type S protein (e.g., amino acids 1214-1273 of SEQ ID NO: 1). In some embodiments, the modified S protein does not comprise the cytoplasmic region of a wild-type S protein (e.g., amino acids 1238-1273 of SEQ ID NO: 1).

In some embodiments, the modified S protein comprises 0, 1, 2, 3, 4, or 5 (e.g., 2) amino acid mutations (e.g., to the proline residue). In some embodiments, the amino acid residue corresponding to position 986 and/or position 987 of SEQ ID NO: 1 are mutated to proline. In some embodiments, the amino acid mutations are K986P and/or V987P.

In some embodiments, the modified S protein does not comprise the fusion peptide domain (e.g., amino acids 788-806 of SEQ ID NO: 1).

In some embodiments, the modified S protein comprises a S1/S2 cleavage site comprising an amino acid sequence that is least 50%, 75%, or 100% identical to GGSG. In some embodiments, the S1/S2 cleavage site corresponds to amino acids 682-685 of SEQ ID NO: 1. In some embodiments, the S1/S2 cleavage site residues RRAR are mutated to GGSG.

In some embodiments, the modified S protein comprises a S2 cleavage site comprising an amino acid sequence that is at least 50%, or 100% identical to AN. In some embodiments, the S2 cleavage site corresponds to amino acids 814 and 815 of SEQ ID NO: 1. In some embodiments, the S2 cleavage site residues KR are mutated to AN.

In some embodiments, mutations at the S1/S2 cleavage site and/or the S2 cleavage site increases the stability of the modified S protein by least or about 10%, 20%, 30%, 40%, 50%, 60%, 70%, 80%, 90%, 100%, or more, as compared to the stability of a wild-type S protein.

In some embodiments, the modified S protein comprises a sequence that is at least or about 70%, 75%, 80%, 85%, 90%, 95%, 96%, 97%, 98%, 99%, or 100% identical to an amino acid sequence selected from the group consisting of SEQ ID NOs: 3-38 and 56-58.

In some embodiments, the modified S protein comprises a sequence that is at least or about 70%, 75%, 80%, 85%, 90%, 95%, 96%, 97%, 98%, 99%, or 100% identical to SEQ ID NO: 29, SEQ ID NO: 32, SEQ ID NO: 35, and/or SEQ ID NO: 38. In some embodiments, the modified S protein comprises a sequence that is at least or about 70%, 75%, 80%, 85%, 90%, 95%, 96%, 97%, 98%, 99%, or 100% identical to SEQ ID NO: 56, SEQ ID NO: 57, SEQ ID NO: 58, and/or SEQ ID NO: 59.

In some embodiments, the modified S protein comprises an immunoglobulin heavy chain variable region (IGVH) signal sequence. In some embodiments, the IGVH signal sequence comprises a sequence that is at least or about 80%, 85%, 90%, 95%, 96%, 97%, 98%, 99%, or 100% identical to SEQ ID NO: 39.

In some embodiments, the modified S protein comprises a linker sequence fused with T4 phage fibritin trimer motif. In some embodiments, the linker sequence comprises a sequence that is at least or about 80%, 85%, 90%, 95%, 96%, 97%, 98%, 99%, or 100% identical to SAIG. In some embodiments, the T4 phage fibritin trimer motif comprises a sequence that is at least or about 80%, 85%, 90%, 95%, 96%, 97%, 98%, 99%, or 100% identical to SEQ ID NO: 40.

In one aspect, the disclosure is related to a nucleic acid (e.g., mRNA) encoding the modified S protein described herein. In some embodiments, the modified S protein is in pre-fusion form. In some embodiments, the modified S protein is in post-fusion form.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a schematic diagram showing domains of SARS-CoV-2 S protein.

FIG. 1B shows 3D schematic structures of pre-fusion and post-fusion forms of SARS-CoV-2 S protein.

FIG. 2 is a schematic diagram showing the DNA plasmid template for in vitro transcription.

FIG. 3 shows the transcription starting site.

FIG. 4 shows gel electrophoresis results of in vitro transcribed mRNAs. The mRNA sequences were SEQ ID NO: 44 (NO.44), SEQ ID NO: 45 (NO. 45), SEQ ID NO: 46 (NO. 46), SEQ ID NO: 47 (NO. 47), and SEQ ID NO: 48 (NO. 48).

FIGS. 5A-5B show Western blot results of wild-type S protein (control) and S protein in pre-fusion form expressed in 293T cells.

FIG. 6 shows spot counts of T lymphocytes secreting INFγ in the spleen of vaccinated mice, measured ELISPOT. The mice were vaccinated with LNP preparations with different mRNA sequences.

FIG. 7 shows spot counts of T lymphocytes secreting INFγ in the spleen of vaccinated mice, measured ELISPOT. The mice were vaccinated with chemically modified mRNA LNP preparations.

FIG. 8 shows SARS-CoV2 S protein-specific IgG antibody titers in mice vaccinated with chemically modified mRNA LNP preparations. The immunization doses were 4 μg, or 50 μg.

FIG. 9 shows SARS-CoV2 S protein-specific IgG antibody titers in mice vaccinated with 4 μg, or 50 μg vaccine preparations.

FIG. 10 shows SARS-CoV2 S protein-specific IgG antibody titers in mice vaccinated with 4 μg, or 50 μg vaccine preparations. The titers were measured by ELISA in serum collected 1 month or 3 months after the booster immunization.

FIG. 11 shows SARS-CoV2 S protein-specific IgG antibody titers in vaccinated BALB/c mice, C57BL/6J mice and B6C3F1 mice. The immunization doses were 1 μg, 5 μg, or 20 μg.

FIG. 12A shows spot counts of T lymphocytes secreting IL2, INFγ, IL4, or IL5 in vaccinated mice. The immunization doses were 1 μg, 5 μg, or 20 μg.

FIG. 12B shows SARS-CoV2 S protein-specific antibody titers of IgG1 and IgG2a in vaccinated mice (**=p-value <0.01, ***=p-value <0.001).

FIG. 13A shows average body weight of male rats in solvent control group (control), and mRNA vaccine group after vaccine administration.

FIG. 13B shows average body weight of female rats in solvent control group (control), and mRNA vaccine group after vaccine administration.

FIG. 14A shows average food intake of male rats in solvent control group (control), and mRNA vaccine group after vaccine administration.

FIG. 14B shows average food intake of female rats in solvent control group (control), and mRNA vaccine group after vaccine administration.

FIG. 15 shows relative organ weight results in vaccinated rats.

FIG. 16 shows histopathological results of acute toxicity test of mRNA vaccine in rats.

FIG. 17 shows non-denaturing Western blot detection results of wild-type S protein (wild type S) and S protein in pre-fusion stable form (S-Trimer) expressed in HEK293T cells. The untransfected group was used as a control.

FIG. 18 lists sequences described in the disclosure.

### DETAILED DESCRIPTION

SARS-CoV-2 is a newly emergent coronavirus that causes COVID-19, which has adversely impacted human health and has led to a pandemic. There is an unmet need to develop vaccines and therapies against SARS-CoV-2 due to its severity and lack of treatment options.

The SARS-CoV-2 virion consists of a helical capsid formed by nucleocapsid (N) proteins bound to the RNA genome, which is enclosed by membrane (M) proteins, envelope (E) proteins and trimeric spike (S) proteins that render them their “corona-like” appearance (See Zhou P, Yang X L, Wang X G, et al. A pneumonia outbreak associated with a new coronavirus of probable bat origin. Nature. 2020 March; 579(7798):270-273). The S protein receptor binding domain (RBD) in the S1 subunit binds to the angiotensin converting enzyme (Angiotensin I Converting Enzyme 2; ACE2) on the cell membranes of type 2 pneumocytes and intestinal epithelial cells. Following binding, the S protein is cleaved by host cell transmembrane serine protease 2 (TMPRSS2), which facilitates subsequent viral entry into host cell (See Hoffmann M, Kleine-Weber H, Schroeder S, et al. SARS-CoV-2 Cell Entry Depends on ACE2 and TMPRSS2 and Is Blocked by a Clinically Proven Protease Inhibitor. Cell. 2020 Mar. 4).

SARS-CoV2 is extremely contagious and can rapidly spread to cause mild to severe infection, including death. Symptoms of COVID-19 can vary. But the two most common symptoms are fever and dry cough. Among those who develop symptoms, approximately one in five may become more seriously ill and have difficulty breathing. Emergency symptoms include difficulty breathing, persistent chest pain or pressure, sudden confusion, difficulty waking, and bluish face or lips; immediate medical attention is advised if these symptoms are present. Further development of the disease can lead to complications including pneumonia, acute respiratory distress syndrome, sepsis, septic shock, and kidney failure.

As is common with most infections, there is a delay for developing symptoms. This period is known as the incubation period, between the moment a person first becomes infected and the appearance of the first symptoms. The median incubation period for COVID-19 is four to five days. Most symptomatic people experience symptoms within two to seven days after exposure, and almost all symptomatic people will experience one or more symptoms before day twelve.

The present disclosure relates to coronavirus vaccines and uses thereof. In one aspect, the disclosure provides a nucleic acid vaccine, comprising a sequence encoding a spike protein or fragment thereof derived from a coronavirus. The present disclosure further shows that these nucleic acid vaccines can successfully induce immune response against coronavirus (e.g., generating neutralizing antibodies).

### Coronavirus Spike Protein

Coronavirus is an enveloped single-stranded positive-stranded RNA virus with the largest RNA virus genome, up to 27-32 kb, which can be divided into 4 genera, i.e., α, β, γ, and δ. Mammals are susceptible to the α and β genera, and γ and δ genera mainly infect poultry. So far, 7 human coronaviruses have been discovered. Among them, the deadly SARS-CoV-2, SARS-CoV and MERS-CoV viruses are categorized into to the β-coronavirus genus of the coronavirus family. The SARS-CoV-2 genome sequence is 29903 bp in length, with 79.5% identity with SARS-CoV gene sequence and 40% identity with MERS-CoV sequence. The main structure includes single-stranded positive-strand nucleic acid (ssRNA), spike protein (S), membrane protein (M), envelope protein (E), and nucelocapsid protein (N). Similar to other β-coronaviruses, the attachment and invasion process of SARS-CoV-2 virus mainly relies on the S protein. The S protein is assembled in the form of a homo-trimer, with a short cytoplasmic tail and a hydrophobic transmembrane domain, and the protein can be anchored into the cell membrane.

The S protein receptor binding domain (RBD) in the 51 subunit binds to the angiotensin converting enzyme (Angiotensin I Converting Enzyme 2; ACE2) on the cell membranes of type 2 pneumocytes and intestinal epithelial cells. Following binding, the S protein is cleaved by host cell transmembrane serine protease 2 (TMPRSS2), which facilitates subsequent viral entry into host cell (See Hoffmann M, Kleine-Weber H, Schroeder S, et al. SARS-CoV-2 Cell Entry Depends on ACE2 and TMPRSS2 and Is Blocked by a Clinically Proven Protease Inhibitor. Cell. 2020 Mar. 4).

As the trimeric spike (S) proteins that protrude through the envelope of the SARS-CoV-2 virion mediates virus entry into the host cells by interacting with the ACE2 human receptor, the major target for anti-SARS-CoV-2 neutralizing antibodies in development are to block the interaction of SARS-CoV-2 S1 protein with ACE2. This S protein is also a target for developing vaccines.

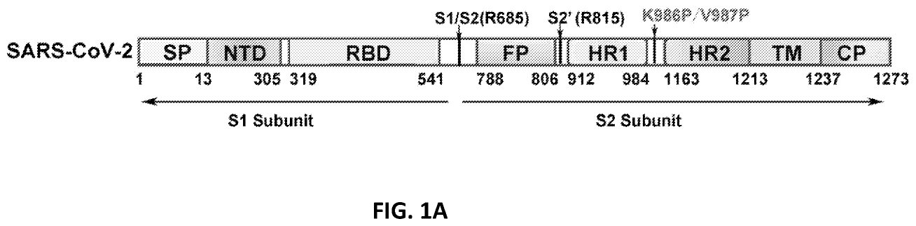

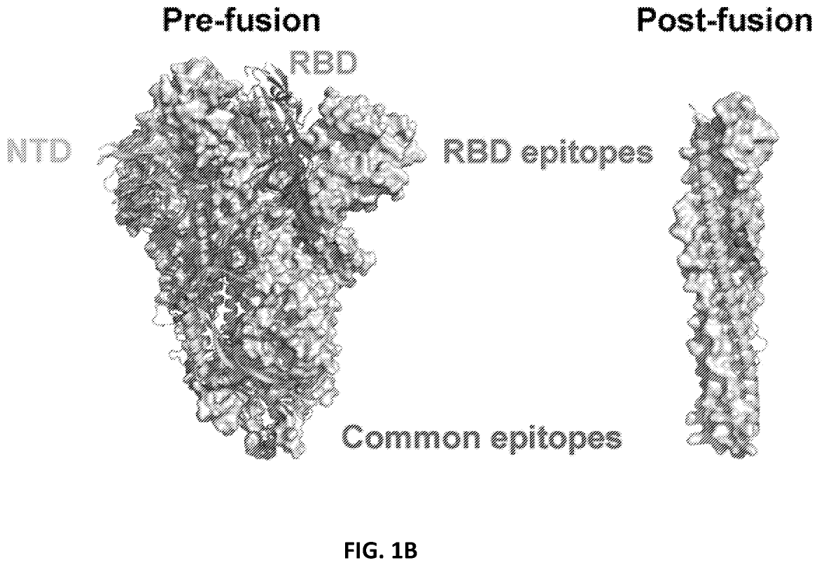

As shown in FIG. 1A, S protein (e.g., SEQ ID NO: 1) can be divided into receptor binding subunit 51 and membrane fusion subunit S2. The 51 subunit can be further divided into a signal peptide (SP), a N-terminal domain (NTD) and a receptor binding domain (RBD). The S2 subunit is anchored to the cell membrane through the transmembrane region. The S2 subunit contains the basic elements required for the membrane fusion process, including: an intrinsic membrane fusion peptide (FP), two heptad repeats (HR), a transmembrane domain (TM), and a C-terminal cytoplasmic domain (CP). After analyzing the pre-fusion structure of the S protein, it was found that the RBD domain of the 51 subunit undergoes a hinge-like conformational movement to hide or expose the key sites of receptor binding. When the RBD domain faces “down”, the receptor cannot bind (“closed”). When the RBD faces “up”, the S protein is in a relatively unstable state where the receptor can bind (“open”). This unstable conformation allows the S protein to easily bind to the host receptor angiotensin converting enzyme 2 (ACE2). When RBD binds to the receptor, the S2 subunit changes to the post-fusion conformation by inserting FP domain into the host cell membrane. HR1 and HR2 form an anti-parallel six-helix bundle (6HB), which together form a fusion core, and ultimately results in fusion of the viral membrane and host cell membrane. With the development of cryo-electron microscopy, a large number of trimeric glycosylated S protein domains have been identified in the pre-fusion conformation. The pre-fusion S protein retains a large number of neutralizing antibody sensitive epitopes, while the exposure of sensitive epitopes is minimized in the post-fusion conformation. Therefore, it is critical to design vaccines (e.g., mRNA vaccines) to induce expression of the S protein in pre-fusion confirmation, which retains the sensitive epitopes, and can induce antibody production to inhibit virus fusion.

mRNA vaccine is a new type of vaccine. The mRNA vaccine can deliver mRNA to cells in the body and express antigen proteins in vivo. The expressed antigen proteins can stimulate humoral immune system to generate antibodies against the antigen proteins, thereby preventing or treating diseases (e.g., COVID-19). Compared with traditional vaccines, advantages of mRNA vaccines include but not limited to: relatively short development and production time; no risk of infection caused by traditional vaccines; and no risk of genome integration of DNA vaccines. In addition, the production of antigens in vivo is usually more effective and convenient. Therefore, mRNA vaccines can be administered to patients to quickly control disease development and spreading.

In some aspects, the disclosure provides mRNAs and mRNA vaccines against SARS-CoV-2 virus. In some embodiments, the mRNAs described herein encode a spike protein or fragment thereof in per-fusion state. In some embodiments, the spike protein or fragment thereof can form a stable trimer.

### Modified Spike Proteins or Fragments Thereof

The disclosure is related to modified spike proteins or fragments thereof derived from a coronavirus (e.g., SARS-CoV-2). In some embodiments, the disclosure is related to a modified coronavirus spike protein or fragment thereof. In some embodiments, the coronavirus is SARS-CoV-2. In some embodiments, the modified spike protein or fragment thereof is locked in a pre-fusion state. In some embodiments, the modified spike protein or fragment thereof is not locked in a pre-fusion state. In some embodiments, the modified-spike protein or fragment thereof is in a stabilized pre-fusion state. In some embodiments, the modifications can inhibit the conversion from a pre-fusion state to a post-fusion state. In some embodiments, the modified-spike protein or fragment thereof is in a post-fusion state. In some embodiments, the RBD domain of the S1 subunit undergoes a hinge-like conformational movement to hide or expose the key sites of receptor binding and the receptor cannot bind to the RBD (“closed”). In some embodiments, the modified-spike protein or fragment thereof is locked or stabilized in this closed pre-fusion conformation. In some embodiments, the RBD faces “up” and exposes the key sites of receptor binding (“open”). In some embodiments, the modified-spike protein or fragment thereof is locked or stabilized in this open pre-fusion conformation.

In some embodiments, the modified spike protein or fragment thereof comprises an extracellular domain, a transmembrane domain, and a C-terminal cytoplasmic region. In some embodiments, the extracellular domain comprises a signal peptide, an N-terminal domain, a receptor-binding domain, a fusion peptide domain, and two heptad repeats. In some embodiments, the modified spike protein or fragment thereof only consists of extracellular domain.

In some embodiments, the modified spike protein or fragment thereof comprises or consists of an extracellular domain that corresponds to amino acids 1-1213 of a wild-type S protein (SEQ ID NO: 1). In some embodiments, the extracellular domain comprises an amino acid sequence that is at least 70%, 75%, 80%, 85%, 90%, 95%, 96%, 97%, 98%, 99%, or 100% identical to amino acids 1-1213 of SEQ ID NO: 1.

In some embodiments, the amino acid in the modified spike protein or fragment thereof that corresponds to K986 of SEQ ID NO: 1 is not Lys. In some embodiments, the amino acid in the modified spike protein or fragment thereof that corresponds to K986 of SEQ ID NO: 1 is a negatively charged amino acid, e.g., Gly, Asp or Glu. In some embodiments, the amino acid in the modified spike protein or fragment thereof that corresponds to K986 of SEQ ID NO: 1 is proline. In some embodiments, the amino acid in the modified spike protein or fragment thereof that corresponds to K986 of SEQ ID NO: 1 is a proline-like unnatural amino acid. In some embodiments, the amino acid in the modified spike protein or fragment thereof that corresponds to V987 of SEQ ID NO: 1 is not Val. In some embodiments, the amino acid in the modified spike protein or fragment thereof that corresponds to V987 of SEQ ID NO: 1 is an amino acid with a large side chain, e.g., Phe, Try, or Trp. In some embodiments, the amino acid in the modified spike protein or fragment thereof that corresponds to V987 is proline. In some embodiments, the amino acid in the modified spike protein or fragment thereof that corresponds to V987 is a proline-like unnatural amino acid.

In some embodiments, the modified spike protein or fragment thereof does not have the fusion peptide domain. In some embodiments, the fusion peptide domain corresponds to amino acids 788-806 of a wild-type S protein (SEQ ID NO: 1).

In some embodiments, the modified spike protein or fragment thereof does not have the C-terminal cytoplasmic region. In some embodiments, the C-terminal cytoplasmic region corresponds to amino acids 1238-1273 of a wild-type S protein (SEQ ID NO: 1). In some embodiments, the modified spike protein or fragment thereof comprises a transmembrane domain. In some embodiments, the transmembrane domain corresponds to amino acids 1214-1237 of a wild-type S protein (SEQ ID NO: 1). In some embodiments, the modified spike protein or fragment thereof comprises an amino acid sequence that is at least 70%, 75%, 80%, 85%, 90%, 95%, 96%, 97%, 98%, 99%, or 100% identical to amino acids 1-1237 of SEQ ID NO: 1.

In some embodiments, the amino acids in the modified spike protein or fragment thereof that correspond to the S1/S2 cleavage site are resistant to proteases (e.g., Furin-like proteases or lysosomal proteases). In some embodiments, the amino acids in the modified spike protein or fragment thereof that corresponds to amino acids 682-685 of SEQ ID NO: 1 are not RRAR. In some embodiments, the amino acids in the modified spike protein or fragment thereof that corresponds to amino acids 682-685 of SEQ ID NO: 1 are GGSG (SEQ ID NO: 53).

In some embodiments, the amino acids in the modified spike protein or fragment thereof that correspond to the S2 cleavage site are resistant to proteases (e.g., Furin-like proteases or lysosomal proteases). In some embodiments, the amino acids in the modified spike protein or fragment thereof that corresponds to amino acids 814 and 815 of SEQ ID NO: 1 are not KR. In some embodiments, the amino acids in the modified spike protein or fragment thereof that corresponds to amino acids 814 and 815 of SEQ ID NO: 1 are AN.

In some embodiments, the signal peptide in the modified spike protein or fragment thereof that corresponds to amino acids 1-13 of SEQ ID NO: 1 is replaced with an immunoglobulin heavy chain variable region signal peptide. In some embodiments, sequence of the immunoglobulin heavy chain variable region signal peptide is at least or about 70%, 75%, 80%, 85%, 90%, 95%, 96%, 97%, 98%, 99%, or 100% identical to SEQ ID NO: 39.

In some embodiments, the modified spike protein or fragment thereof comprises a T4 phage fibritin trimer motif. In some embodiments, sequence of the T4 phage fibritin trimer motif is at least or about 70%, 75%, 80%, 85%, 90%, 95%, 96%, 97%, 98%, 99%, or 100% identical to SEQ ID NO: 40. In some embodiments, the T4 phage fibritin trimer motif is linked to the rest of the modified spike protein or fragment thereof via a linker peptide. In some embodiments, the linker peptide comprises a sequence that is at least or about 70%, 75%, 80%, 85%, 90%, 95%, 96%, 97%, 98%, 99%, or 100% identical to SAIG (SEQ ID NO: 54).

In one aspect, the disclosure is related to a protein complex comprising the spike protein or fragment thereof described herein. In some embodiments, the protein complex comprise three spike proteins or fragments thereof described herein. In some embodiments, the three spike proteins or fragments thereof are the same. In some embodiments, the three spike proteins or fragments thereof are different.

In some embodiments, the modified spike protein or fragment thereof comprises a sequence that is at least 70%, 75%, 80%, 85%, 90%, 91%, 92%, 93%, 94%, 95%, 96%, 97%, 98%, 99%, or 100% identical to any one of SEQ ID NOs: 1, 3-38, and 56-59. In some embodiments, the modified spike protein or fragment thereof comprises a sequence that is at least 70%, 75%, 80%, 85%, 90%, 91%, 92%, 93%, 94%, 95%, 96%, 97%, 98%, 99%, or 100% identical to any one of SEQ ID NOs: 3-38 and 56-59 without amino acids 1-13. In some embodiments, the modified spike protein or fragment thereof comprises a sequence that is at least 70%, 75%, 80%, 85%, 90%, 91%, 92%, 93%, 94%, 95%, 96%, 97%, 98%, 99%, or 100% identical to amino acids 14-1213 of SEQ ID NO: 29. In some embodiments, the modified spike protein or fragment thereof comprises a sequence that is at least 70%, 75%, 80%, 85%, 90%, 91%, 92%, 93%, 94%, 95%, 96%, 97%, 98%, 99%, or 100% identical to amino acids 14-1194 of SEQ ID NO: 32. In some embodiments, the modified spike protein or fragment thereof comprises a sequence that is at least 70%, 75%, 80%, 85%, 90%, 91%, 92%, 93%, 94%, 95%, 96%, 97%, 98%, 99%, or 100% identical to amino acids 14-1237 of SEQ ID NO: 35. In some embodiments, the modified spike protein or fragment thereof comprises a sequence that is at least 70%, 75%, 80%, 85%, 90%, 91%, 92%, 93%, 94%, 95%, 96%, 97%, 98%, 99%, or 100% identical to amino acids 14-1218 of SEQ ID NO: 38. In some embodiments, the modified spike protein or fragment thereof further comprises an immunoglobulin heavy chain variable region signal peptide.

### Nucleic Acid Encoding the Spike Protein or Fragment Thereof

In some embodiments, the disclosure is related to a nucleic acid encoding the spike protein or fragment thereof described herein. As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof. In some embodiments, the nucleic acid includes ribonucleotides, deoxyribonucleotides, and/or unnatural nucleotides. In some embodiments, it can be recognized and served as a template for a polymerase (DNA polymerase or RNA polymerase). In some embodiments, it can be recognized and translated into a polypeptide by a ribosome.

In some embodiments, the nucleic acid described herein comprises a 5′ UTR (untranslated region). In some embodiments, the 5′ UTR is a hydroxysteroid (17-β) dehydrogenase (HSD17B4) gene 5′-UTR sequence or fragment thereof. In some embodiments, the 5′ UTR comprises a sequence that is at least or about 70%, 75%, 80%, 85%, 90%, 91%, 92%, 93%, 94%, 95%, 96%, 97%, 98%, 99%, or 100% identical to SEQ ID NO: 43. In some embodiments, the nucleic acid described herein comprises a 3′ UTR. In some embodiments, the 3′ UTR is a serum albumin gene (ALB) 3′-UTR sequence or fragment thereof. In some embodiments, the 3′ UTR comprises a sequence that is at least or about 70%, 75%, 80%, 85%, 90%, 91%, 92%, 93%, 94%, 95%, 96%, 97%, 98%, 99%, or 100% identical to SEQ ID NO: 43. In some embodiments, the nucleic acid described herein comprises a Kozak sequence. In some embodiments, the Kozak sequence comprises a sequence that is at least or about 70%, 75%, 80%, 85%, 90%, 91%, 92%, 93%, 94%, 95%, 96%, 97%, 98%, 99%, or 100% identical to SEQ ID NO: 42. In some embodiments, the nucleic acid described herein comprises a polyA tail. In some embodiments, the polyA tail comprises at least or about 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 150, 160, 170, 180, 190, or 200 adenosines.

In some embodiments, the nucleic acid described herein comprises from 5′ end to 3′ end the following elements: the 5′ UTR described herein; the Kozak sequence described herein; an open reading frame (ORF) encoding the modified spike protein or fragment thereof described herein; the 3′ UTR described herein; and the polyA tail described herein.

In some embodiments, the nucleic acid described herein comprises a multiple cloning site (MSC) between the stop codon (e.g., UGA) of the open reading frame (ORF) described herein and the 3′ UTR. In some embodiments, the multiple cloning site comprises at least or about 5, 6, 7, 8, 9, 10, 11, 12, 13, 14, 15, 16, 17, 18, 19, or 20 nucleotides In some embodiments, the multiple cloning site is about or at least 70%, 75%, 80%, 85%, 90%, 91%, 92%, 93%, 94%, 95%, 96%, 97%, 98%, 99%, or 100% identical to CUCGAGACUAGU (SEQ ID NO: 55).

In some embodiments, the nucleic acid described herein comprises a sequence that is at least or about 70%, 75%, 80%, 85%, 90%, 91%, 92%, 93%, 94%, 95%, 96%, 97%, 98%, 99%, or 100% identical to any one of SEQ ID NOs: 2 and 44-52. In some embodiments, the nucleic acid described herein comprises a sequence that is at least or about 70%, 75%, 80%, 85%, 90%, 91%, 92%, 93%, 94%, 95%, 96%, 97%, 98%, 99%, or 100% identical to SEQ ID NO: 44 or 49. In some embodiments, the nucleic acid described herein comprises a sequence that is at least or about 70%, 75%, 80%, 85%, 90%, 91%, 92%, 93%, 94%, 95%, 96%, 97%, 98%, 99%, or 100% identical to SEQ ID NO: 45 or 50. In some embodiments, the nucleic acid described herein comprises a sequence that is at least or about 70%, 75%, 80%, 85%, 90%, 91%, 92%, 93%, 94%, 95%, 96%, 97%, 98%, 99%, or 100% identical to SEQ ID NO: 46 or 51. In some embodiments, the nucleic acid described herein comprises a sequence that is at least or about 70%, 75%, 80%, 85%, 90%, 91%, 92%, 93%, 94%, 95%, 96%, 97%, 98%, 99%, or 100% identical to SEQ ID NO: 47 or 52.

In some embodiments, the nucleic acid described herein is a DNA molecule. In some embodiments, the nucleic acid described herein is an RNA molecule. In some embodiments, the RNA molecule is an mRNA molecule. In some embodiments, the RNA molecule is an mRNA molecule with modified nucleotides.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of illustration, the comparison of sequences and determination of percent identity between two sequences can be accomplished, e.g., using a Blossum 62 scoring matrix with a gap penalty of 12, a gap extend penalty of 4, and a frameshift gap penalty of 5.

Preparation of mRNA

In some embodiments, the disclosure is related to a recombinant vector (e.g., a plasmid that can be linearized as a DNA template for in vitro transcription) comprising any of the nucleic acid described herein. In some embodiments, the vector comprises a promoter (e.g., T7 promoter). In some embodiments, the T7 promoter comprises a sequence that is at least or about 70%, 75%, 80%, 85%, 90%, 91%, 92%, 93%, 94%, 95%, 96%, 97%, 98%, 99%, or 100% identical to TAATACGACTCACTATA. In some embodiments, the three nucleotides immediately following the promoter are AGG, GGG, or CGG. In some embodiments, the vector comprises one or more linearization sites, e.g., following the polyA tail described herein. In some embodiments, the one or more linearization sites comprises a restriction enzyme digestion site, e.g., XbaI or EcoRI

In some embodiments, the nucleic acid described herein is an mRNA. In some embodiments, the mRNA is synthesized by in vitro transcription. In some embodiments, the mRNA comprises from 5′ to 3′ direction: a 5′ cap, a 5′-UTR, an open reading frame (ORF), a 3′-UTR, and a polyA tail. In some embodiments, the mRNA comprises a sequence that is at least or about 70%, 75%, 80%, 85%, 90%, 95%, 96%, 97%, 98%, 99%, or 100% identical to SEQ ID NO: 44, SEQ ID NO: 45, SEQ ID NO: 46, and/or SEQ ID NO: 47. In some embodiments, the mRNA comprises a sequence that is at least or about 70%, 75%, 80%, 85%, 90%, 95%, 96%, 97%, 98%, 99%, or 100% identical to SEQ ID NO: 49, SEQ ID NO: 50, SEQ ID NO: 51, and/or SEQ ID NO: 52.

In some embodiments, the 5′cap have a structure of m⁷G5′ppp5′(2′-OMe)NpG, in which N is any nucleoside. In some embodiments, N is adenosine (A) or m⁶A (N⁶-Methyladenosine). In some embodiments, the 5′cap increases mRNA stability by at least or about 10%, 20%, 30%, 40%, 50%, 1 fold, 2 folds, 5 folds, 10 folds, 20 folds, 50 folds, or 100 folds as compared to an mRNA without the 5′ cap. In some embodiments, the 5′ cap increases mRNA translation efficiency by at least or about 10%, 20%, 30%, 40%, 50% as compared to an mRNA without the 5′ cap. In some embodiments, the 5′cap decreases mRNA degradation (e.g., by exonuclease) to less than 50%, less than 40%, less than 30%, less than 20%, less than 10%, less than 5%, less than 3%, less than 2%, or less than 1% as compared that of an mRNA without the 5′ cap.

In some embodiments, the mRNA comprises a 5′-UTR. In some embodiments, the 5′-UTR is a hydroxysteroid (17-β) dehydrogenase (HSD17B4) gene 5′-UTR sequence fragment (SEQ ID NO. 41), and/or a Kozak sequence (SEQ ID NO. 42). In some embodiments, the 5′-UTR is the 5′-UTR of HBB (Hemoglobin Subunit Beta), Hsp70 (70 kilodalton heat shock proteins), DNAH2 (Dynein Axonemal Heavy Chain 2), or HSD17B4.

In some embodiments, the 5′ UTR described herein decreases effects from cis-acting destabilizing sequences and/or upstream promoter sequences by at least or about 10%, 20%, 30%, 40%, or 50% as compared to those of an mRNA without the 5′ UTR.

In some embodiments, the mRNA comprises a 3′-UTR. In some embodiments, the 3′-UTR is a 3′-UTR of a homolog, fragment or variant thereof derived from the any one of the following genes: albumin gene, α-globin gene, β-globin gene, tyrosine hydroxylase gene, heat shock protein 70 gene, lipoxygenase gene and/or collagen alpha gene. In some embodiments, the 3′-UTR is the serum albumin gene (ALB) 3′-UTR sequence (SEQ ID NO. 43). In some embodiments, the 3′ UTR described herein increases stability (e.g., half-life) of the mRNA by at least or about 10%, 20%, 30%, 40%, 50%, 1 fold, 2 folds, 5 folds, 10 folds, 20 folds, 50 folds, or 100 folds as compared to that of an mRNA without the 3′UTR.

In some embodiments, the mRNA comprises a polyA tail. In some embodiments, the polyA tail comprises about 100-200 adenosines. In some embodiments, the polyA tail comprises about 100, 110, 120, 130, 140, 150, 160, 170, 180, 190, or 200 adenosines. In some embodiments, the polyA tail comprises about 120 adenosines. In some embodiments, the polyA tail decreases mRNA degradation (e.g., by exonuclease) to less than 50%, less than 40%, less than 30%, less than 20%, less than 10%, less than 5%, less than 3%, less than 2%, or less than 1% as compared that of an mRNA without the polyA tail.

In some embodiments, the mRNA is modified, e.g., to include naturally occurring and/or chemically-modified nucleoside. In some embodiments, the mRNA includes one or more naturally occurring nucleosides, e.g., pseudouridine, 2-thiouridine, 5-methyluridine, 5-methylcytidine, and/or N6-methyladenosine. In some embodiments, the mRNA includes one or more chemically-modified nucleosides, e.g., N1-methylpseudouridine, and/or 5-ethynyluridine. In some embodiments, the ratio of rUTP to N1-methylpseudouridine (1-N-Me-Pseudo-UTP) in the modified mRNA is from 2:1 to 1:2, e.g. between 1.5:1 and 1:1.5, between 1.2:1 and 1:1.2, between 1.1:1 and 1:1.1, or roughly about 1:1.

In some embodiments, the mRNA comprises one or more modified nucleosides. In some embodiments, the mRNA comprises one or more replacements of UTP with pseudo-UTP, N1-methyl-pseudo-UTP, rUTP, and/or 5-ethynyl-UTP. In some embodiments, the mRNA comprises one or more replacements of CTP with 5-methyl-CTP (5-Me-CTP). In some embodiments, the mRNA comprises one or more replacements of ATP with m⁶ATP. In some embodiments, the replacements described herein occur during mRNA synthesis, e.g., in vitro transcription.

In some embodiments, the mRNA comprises one or more chemically-modified nucleosides. In some embodiments, the one or more chemically-modified nucleosides increases stability (e.g., half-life) of the mRNA by at least or about 10%, 20%, 30%, 40%, 50%, 1 fold, 2 folds, 5 folds, 10 folds, 20 folds, 50 folds, or 100 folds as compared to that of an mRNA without the one or more chemically-modified nucleosides. In some embodiments, the one or more chemically-modified nucleosides increase cell-mediated immune response (e.g., INFγ release) more than or about 90%, 85%, 80%, 75%, 70%, 65%, 60%, 55%, or 50% as compared to that of an mRNA without the one or more chemically-modified nucleosides.

### Preparation of Vaccine Against Coronavirus

In some embodiments, the disclosure is related to a vaccine against coronavirus (e.g., SARS-CoV-2). In some embodiments, the vaccine comprises any of the nucleic acids (e.g., mRNA) described herein. In some embodiments, the vaccine comprises any of the spike proteins or fragments thereof described herein. In some embodiments, the vaccine comprises any of the protein complexes described herein.

In some embodiments, the vaccine is a nucleic acid vaccine. In some embodiments, the vaccine is an mRNA vaccine. In some embodiments, the mRNA vaccine comprises one or more of the mRNAs described herein. In some embodiments, the mRNA vaccine induces antibody (e.g., IgG) production against the spike protein or fragment described herein.

In some embodiments, the mRNA vaccine comprises a lipid nanoparticle (LNP). In some embodiments, the lipid nanoparticle encapsulates the any of the mRNAs described herein. In some embodiments, the lipid nanoparticle is a liposome. In some embodiments, the lipid nanoparticle comprises one or more of: (1) ionizable lipids, (2) PEGylated lipids (Lipid-PEG) or modified PEG-Lipid, (3) cholesterol and derivatives thereof, (4) phospholipids.

In some embodiments, the disclosure is related to a method to prepare an mRNA vaccine against coronavirus (e.g., SARS-CoV-2), comprising: a) mixing ionizable lipids, phospholipids, cholesterol and derivatives thereof, and/or PEGylated lipids to obtain the lipid nanoparticles described herein; b) mixing the mRNA described herein with the lipid nanoparticles using a microfluidic mixer; c) dialyzing and ultrafiltrating the mixture in step b; d) filtering the mixture through a membrane with an appropriate size.

In some embodiments, the ratio of different lipids, including but not limited to ionizable lipids, phospholipids, cholesterol and derivatives thereof, and/or PEGylated lipids is optimized. In some embodiments, the lipid nanoparticles are obtained by mixing the mixture of different lipids with the mRNA described herein in a microfluidic mixer. Parameters during the mixing include: ratio of the phosphate and nitrogen, flow rate, and/or flow velocity. In some embodiments, the lipid nanoparticles encapsulating the mRNA described herein are dialyzed, filtered through a membrane.

### Methods of Administration

In one aspect, the composition described herein induces production of antibody (e.g., IgG) the specifically binds to coronavirus (e.g., SARS-CoV-2). In some embodiments, the vaccine described herein provides immunity to a subject against coronavirus (e.g., SARS-CoV-2). As used herein, when referring to an antibody, the phrases “specifically binding” and “specifically binds” mean that the antibody interacts with its target molecule preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to the S protein may be referred to as an S protein-specific antibody or an anti-S protein antibody.

In one aspect, the disclosure provides methods for treating a coronavirus-related disease in a subject, methods of neutralizing a coronavirus, methods of blocking a coronavirus/ACE2 interaction, methods of inducing Fc-dependent antiviral functions, methods of blocking internalization of a coronavirus by a cell, methods of identifying a subject having a coronavirus-related disease. In some embodiments, the treatment can halt, slow, retard, or inhibit progression of a coronavirus-related disease. In some embodiments, the treatment can result in the reduction of in the number, severity, and/or duration of one or more symptoms of the coronavirus-related disease in a subject.

In one aspect, the disclosure features methods that include administering a therapeutically effective amount of a composition (e.g., polypeptides or nucleic acids) disclosed herein to a subject in need thereof (e.g., a subject at risk of having, vulnerable to, or identified or diagnosed as having, a coronavirus-related disease).

As used herein, the term “subject” refers to an animal, human or non-human, to whom the administration according to the methods of the present disclosure is provided. Veterinary and non-veterinary applications are contemplated in the present disclosure. Human subjects can be adult humans or juvenile humans (e.g., humans below the age of 18 years old). In some embodiment, the human subject is at least 50, 55, 60, 65, or 70 years old. In some embodiment, the human subject is below the age of 1, 2, 3, 4, 5, 6, 7, 8, 9, 10, 11, 12, 13, 14, 15, 16, 17, 18 years old. In addition to humans, subjects include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals. In some embodiments, the subject has a higher risk of developing severe symptoms (e.g., a higher risk of death or being treated by a ventilator) if the subject is infected with coronavirus. In some embodiments, the subject is at least 40, 50, 60, 65, or 70 years old.

As used herein, by an “effective amount” is meant an amount or dosage sufficient to effect beneficial or desired results including halting, slowing, retarding, preventing, or inhibiting progression of a disease, e.g., a coronavirus-related disease. An effective amount will vary depending upon, e.g., an age and a body weight of a subject to which the polypeptide, the polynucleotide, and/or compositions thereof is to be administered, the severity of symptoms and the route of administration, and thus administration can be determined on an individual basis.

In some embodiments, the coronavirus-related disease is COVID-19 (Coronavirus disease 2019), Severe acute respiratory syndrome (SARS), or Middle East respiratory syndrome (MERS). In some embodiments, the coronavirus that causing the coronavirus-related disease is SARS-CoV, SARS-CoV-2, MERS-CoV, or other types of coronavirus having one or more S proteins. In some embodiments, the amino acid sequence of the S protein of the coronavirus described herein comprises a sequence that is at least or about 50%, at least or about 55%, at least or about 60%, at least or about 65%, at least or about 70%, at least or about 75%, at least or about 80%, at least or about 85%, at least or about 90%, at least or about 95%, or at least or about 98% identical to the receptor-biding domain sequence of the SARS-CoV-2 S protein.

In some embodiments, the compositions and methods disclosed herein can be used for treatment of patients at risk of developing a coronavirus-related disease or at risk of being infected with a coronavirus (e.g., a health worker). In some embodiments, the compositions and methods disclosed herein can be administered to a subject that is in the coronavirus incubation period or a subject that is being suspected of having being infected with the coronavirus.

An effective amount can be administered in one or more administrations. By way of example, an effective amount is an amount sufficient to ameliorate, stop, stabilize, reverse, inhibit, slow and/or delay progression of a coronavirus-related disease in a patient. As is understood in the art, an effective amount may vary, depending on, inter alia, patient history as well as other factors such as the type (and/or dosage) of the composition used. In some embodiments, the effective amount will not completely prevent the subject from being infected with the virus, but it can halt, slow, inhibit progression of the disease, or at least reduce the severity of the symptoms. In some embodiments, it can significantly reduce the risk of death or reduce the need of using a ventilator.

Effective amounts and schedules for administering the compositions disclosed herein can be determined empirically. A typical daily dosage of an effective amount of the polypeptide, the nucleic acid, or the composition is 1 μg/dose to 1000 μg/dose. In some embodiments, the dosage can be less than 1000 μg/dose, 900 μg/dose, 800 μg/dose, 700 μg/dose, 600 μg/dose, 500 μg/dose, 400 μg/dose, 300 μg/dose, 200 μg/dose, 100 μg/dose, 50 μg/dose, or 10 μg/dose. In some embodiments, the dosage can be at least 10 μg/dose, 15 μg/dose, 20 μg/dose, 25 μg/dose, 30 μg/dose, 35 μg/dose, 40 μg/dose, 45 μg/dose, 50 μg/dose, 60 μg/dose, 70 μg/dose, 80 μg/dose, 90 μg/dose, 100 μg/dose, 200 μg/dose, or 300 μg/dose. In some embodiments, the dosage is 10-500 μg/dose, 20-500 μg/dose, 25-500 μg/dose, 10-400 μg/dose, 10-300 μg/dose, or 20-400 μg/dose for a human. In some embodiments, the dosage is 25-300 μg/dose for a human.

In any of the methods described herein, at least one dose and, optionally, at least one additional dose can be administered to the subject. In some embodiments, the two doses are separated by at least 1, 2, 3, 4, 5, 6, 7, 8, 9, 10, 11, or 12 weeks. In some embodiments, a third, a fourth, and/or a fifth doses can be administered to the subject. In some embodiments, multiple doses can be administered to the subject over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). In some embodiments, multiple doses are administered to the subject until the subject develops a sufficient immunity against the coronavirus.

While it is desirable that the polypeptide translated from the nucleic acid can induce immune response, it is advantageous that the nucleic acid itself or the composition (e.g., LNP) itself does not induce immune response or has limited immunogenicity. In some embodiments, the vaccine described herein induces limited cell-mediated immune response (e.g., cytokine release and/or inflammatory effects). In some embodiments, the vaccine induces limited cytokine secretion (e.g., IFNγ) by splenic lymphocytes. In some embodiments, IFNγ secretion, as determined by the ELISPOT counts, is more than 500, more than 400, more than 300, more than 200, more than 150, more than 100, more than 95, more than 90, more than 85, more than 80, more than 75, more than 70, more than 65, more than 60, more than 55, more than 50, more than 45, more than 40, more than 35, more than 30, more than 25, more than 20, more than 10, or more than 5, when the vaccine is administered at about 1 μg, 5 μg, 10 μg, or 20 μg to a subject (e.g., a human, a mouse, or a rat). In some embodiments, the ELISPOT assay is performed after 3 days, 4 days, 5 days, after 6 days, after 7 days, after 8 days, after 9 days, after 10 days, after 2 weeks, after 3 weeks, or after 4 weeks, of vaccine administration.

In some embodiments, the vaccine described herein induced humoral immune response (e.g., antibody production). In some embodiments, the vaccine induces IgG production. In some embodiments, the IgG EC50 tier of the vaccine is at least or about 5000, at least or about 10000, at least or about 15000, at least or about 20000, at least or about 25000, at least or about 30000, at least or about 35000, at least or about 40000, at least or about 45000, at least or about 50000, at least or about 55000, at least or about 60000, at least or about 65000, at least or about 70000, at least or about 75000, at least or about 80000, at least or about 85000, at least or about 90000, at least or about 1×10⁵, at least or about 1×10⁶, at least or about 5×10⁶, at least or about 1×10⁷, at least or about 5×10⁷, or at least or about 1×10⁸, e.g., when the vaccine is administered at about 1 μg, 5 μg, 10 μg, or 20 μg to a subject (e.g., a human, a mouse, or a rat). In some embodiments, the IgG EC50 titer are determined after 3 days, 4 days, 5 days, after 6 days, after 7 days, after 8 days, after 9 days, after 10 days, after 2 weeks, after 3 weeks, or after 4 weeks, of vaccine administration.

In some embodiments, the vaccine described herein is administered at a dose level of about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 40 μg, about 50 μg, about 60 μg, about 70 μg, about 80 μg, about 90 μg, about 100 μg, about 150 μg, about 200 μg, about 250 μg, about 300 μg, about 350 μg, about 400 μg, or about 500 μg for a subject (e.g., a human subject).

In some embodiments, the vaccine described herein is administered to a subject (e.g., rat) at a dosage level of about 0.5 mg, about 1 mg, about 1.5 mg. about 2 mg, about 2.5 mg, or about 3 mg. In some embodiments, the vaccine described herein is administered to a subject (e.g., human) at a dosage level of about 1 μg/kg to 6000 μg/kg (μg per kg of subject weight). In some embodiments, the dosage can be less than 1000 μg/kg, 500 μg/kg, 100 μg/kg, 10 μg/kg, 9 μg/kg, 8 μg/kg, 7 μg/kg, 6 μg/kg, 5 μg/kg, 4 μg/kg, 3 μg/kg, 2 μg/kg, 1 μg/kg, 0.5 μg/kg, or 0.1 μg/kg. In some embodiments, the dosage can be at least 10 μg/kg, 9 μg/kg, 8 μg/kg, 7 μg/kg, 6 μg/kg, 5 μg/kg, 4 μg/kg, 3 μg/kg, 2 μg/kg, or 1 μg/kg. In some embodiments, the dosage is about 10 μg/kg, 9 μg/kg, 8 μg/kg, 7 μg/kg, 6 μg/kg, 5 μg/kg, 4 μg/kg, 3 μg/kg, 2 μg/kg, 1 μg/kg, 0.9 μg/kg, 0.8 μg/kg, 0.7 μg/kg, 0.6 μg/kg, 0.5 μg/kg, 0.4 μg/kg, 0.3 μg/kg, 0.2 μg/kg, or 0.1 μg/kg.

In some embodiments, the vaccine described herein is administered by intramuscular, subcutaneous, or intradermal injections. In some embodiments, the vaccine described herein is administered once a month, twice a month, three times a month, or four times a month.

In some embodiments, the subject is a mouse. In some embodiments, the mouse has a BALB/c background, a C57BL/6J background, a B6C3F1 background, or combinations thereof.

In some embodiments, the vaccine described herein induces neutralizing antibody production against coronavirus (e.g., SARS-CoV-2). As used herein, the term “neutralizing antibody” refers to an antibody that is responsible for defending cells from pathogens. In some embodiments, the neutralizing antibody can bind to the RBD and/or block the virus from entering the cell. In some embodiments, the neutralizing antibody has an IC50 titer value of at least or about 50, at least or about 100, at least or about 500, at least or about 1000, at least or about 2000, at least or about 3000, at least or about 4000, or at least or about 5000.

In some embodiments, the vaccine described herein is administered at least once, at least twice, at least three times, at least four times, at least five times, about least six times to induce immune response within a subject. In some embodiments, the antibody (e.g., SARS-CoV-2 S protein specific IgG) collected after 2 months, after 3 months, after 4 months, after 5 months, after 6 months, after 7 months, after 8 months, after 9 months, after 10 months, after 11 months, or after 1 year of the vaccine administration has a titer value (e.g., an endpoint dilution titer) that is at least or about 50%, at least or about 60%, at least or about 70%, at least or about 80%, at least or about 90%, or at least or about 100% to that of the antibody collected after 1 month of the vaccine administration. In some embodiments, the immune response (e.g., as measured by a detectable level of a neutralizing antibody) is maintained for at least 6, 7, 8, 9, 10, 11, 12 moths, or at least 1, 2, 3, 4, 5, or 10 years.

In some embodiments, the vaccine described herein induces Th1 immune response (e.g., INFγ and/or IL2 secretion). In some embodiments, the secretion level of INFγ and/or IL2 is increased by at least or about 10%, 20%, 30%, 40%, 50%, 1 fold, 2 folds, 5 folds, 10 folds, 20 folds, 50 folds, or 100 folds as compared to that when the vaccine is not administered. In some embodiments, IL1 secretion, as determined by the ELISPOT counts, is at least or about 100, 150, 200, 250, 300, 350, 400, 450, or 500, when the vaccine is administered at a dose level of about 1 μg, 5 μg, or 20 μg. In some embodiments, IFN-γ secretion, as determined by the ELISPOT counts, is at least or about 20, 30, 40, 50, 60, 70, 80, 90, or 100, when the vaccine is administered at a dose level of about 1 μg, 5 μg, or 20 μg.

In some embodiments, the IgG2a titer (e.g., an endpoint dilution titer) induced by the vaccine described herein is at least 1×10⁵, at least 1×10⁶, or at least 1×10⁷. In some embodiments, the IgG1 titer (e.g., an endpoint dilution titer) induced by the vaccine described herein is at least 1×10⁴, at least 1×10⁵, or at least 1×10⁶. In some embodiments, the IgG2a titer is at least or about 5%, 10%, 20%, 30%, 40%, 50%, or 60% higher than that of the IgG1 titer induced by the vaccine administered at a dose level of about 1 μg, 5 μg, or 20 μg.

In some embodiments, the body weight of a subject (e.g., a human, a mouse, or a rat), after administration with the vaccine (e.g., mRNA vaccine) described herein for about 5 days, 6 days, 7 days, 8 days, or 9 days, is at least 70%, 80%, 90%, or 100% as compared to that of a subject administered with a solvent control. In some embodiments, administration of the vaccine (e.g., mRNA vaccine) described herein does not affect body weight of the subject.

In some embodiments, the food intake of a subject (e.g., a human, a mouse, or a rat), after administration with the vaccine (e.g., mRNA vaccine) described herein for about 4 days, 5 days, 6 days, 7 days, 8 days, or 9 days, is at least 70%, 80%, 90%, or 100% as compared to that of a subject administered with a solvent control. In some embodiments, administration of the vaccine (e.g., mRNA vaccine) described herein does not affect food intake of the subject.

In some embodiments, the composition as described herein is safe and non-toxic. In some embodiments, the relative organ weight of an organ (e.g., heart, liver, spleen, lung, kidney, thymus, lymph nodes, or brain) of a subject (e.g., a human, a mouse, or a rat), after administration of the vaccine (e.g., mRNA vaccine) described herein, is determined. In some embodiments, the relative organ weight of the heart is about 0.1, about 0.2, about 0.3, or about 0.4. In some embodiments, the relative organ weight of the liver is about 2, about 3, about 4, about 5, or about 6. In some embodiments, the relative organ weight of the spleen is about 0.1, about 0.15, about 0.2, about 0.25, or about 0.3. In some embodiments, the relative organ weight of the lung is about 0.2, about 0.3, about 0.4, or about 0.5. In some embodiments, the relative organ weight of the kidney (e.g., left kidney or right kidney) is about 0.2, about 0.3, about 0.4, or about 0.5. In some embodiments, the relative organ weight of the thymus is about 0.5, about 1, about 1.5, about 2, or about 2.5. In some embodiments, the relative organ weight of the lymph nodes is about 0.002, about 0.004, about 0.006, about 0.008, about 0.01, or about 0.012. In some embodiments, the relative organ weight of the brain is about 0.5, about 0.6, about 0.7, about 0.8, or about 0.9.

In some embodiments, the lesion severity score of an organ (e.g., heart, liver, kidney, spleen, thymus, lymph nodes, pancreas, lung, or muscle tissue) of a subject (e.g., a human, a mouse, or a rat) after administration of the vaccine (e.g., mRNA vaccine) described herein, is 0, 1, 2, 3, 4, or 5. In some embodiments, the lesion severity score of the organ is the same in the subject administered with the vaccine as compared to a subject administered with a solvent control. In some embodiments, the lesion severity score of the organ is increase (e.g., by 1, or 2) in the subject administered with the vaccine as compared to a subject administered with a solvent control.

In some embodiments, one or more immune modulators can be co-administered to a subject to enhance the immune response. The agent can be lipophilic and can be embedded within the lipid nanoparticles. The immune modulator can be a ligand for a Toll Receptor or an adjuvant such as any of those described herein. Ligands for Toll Receptors include any of a variety of microbial molecules (e.g., proteins, nucleic acids, or lipids) such as, but not limited to, triacyl lipopeptides, OspA, Porin PorB, peptidoglycan, lipopolysaccharide (LPS), hemagglutinin, flavolipin, unmethylated CpG DNA, flagellin, lipoarabinomannan, or zymosan. In some embodiments, the adjuvant can be Freund's complete or incomplete adjuvant, alum, RIBI, or similar immunostimulatory agent. Adjuvants can also include, e.g., cholera toxin (CT), E. coli heat labile toxin (LT), mutant CT (MCT) (Yamamoto et al. (1997) J. Exp. Med. 185:1203-1210) and mutant E. coli heat labile toxin (MLT) (Di Tommaso et al. (1996) Infect. Immunity 64:974-979). These immune modulators, adjuvants, and nanoparticles are described in e.g., US20200188475A1, U.S. Ser. No. 10/392,341B2, U.S. Pat. No. 8,802,644B2, the disclosure of which are incorporated herein by reference in its entirety.

### Pharmaceutical Composition

Embodiments herein provide for administration of compositions to subjects in a biologically compatible form suitable for pharmaceutical administration in vivo. By “biologically compatible form suitable for administration in vivo” it is meant a form of the active agent (e.g. any of the nucleic acid described herein, or any of the spike protein or fragment thereof) to be administered in which any toxic or otherwise adverse effects are outweighed by the therapeutic or prophylactic effects of the active agent. Administration of a therapeutically or prophylactically active amount of the therapeutic or prophylactic composition is defined as an amount effective, at dosages and for periods of time necessary to achieve a desired result, including but not limited to increased immunity to a viral pathogen. For example, a therapeutically or prophylactically active amount of a compound can vary according to factors such as the disease state, age, sex, and weight of the individual, and the ability of formulations to elicit a desired response in the individual, including but not limited to a response which boosts immunity to a viral pathogen. Dosage regimen may be adjusted to provide the optimum therapeutic and/or prophylactic response.

In some embodiments, composition (e.g. pharmaceutical chemical, protein, peptide or nucleic acid of an embodiment) can be administered in a convenient manner such as subcutaneous, intravenous, intramuscular, intradermal, by oral administration, inhalation, transdermal application, intravaginal application, topical application, intranasal or rectal administration. In a more particular embodiment, the product can be orally or subcutaneously administered. In another embodiment, the composition can be administered intravenously. In some embodiments, the composition be administered intranasally, such as inhalation. In some embodiments, the composition can be administered intramuscularly. In some embodiments, the composition can be administered intradermally. In some embodiments, the composition can be administered through a respiratory route.

The present disclosure also provides expression vectors comprising or consisting one or more nucleic acids described herein. In some embodiments, the recombinant expression cassette can be placed in an expression vector, such that the nucleic acid segment encoding the peptide can persist through cell divisions. For example, the recombinant expression cassette is a DNA/RNA fragment, and suitable DNA/RNA constructs can be linear or circular constructs configured as an expression vector. Thus, in some embodiment, the expression vector includes a viral vector (e.g., replicating or nonreplicating recombinant adenovirus genome). In some embodiments, these generated recombinant viruses can then be used—individually or in combination—as a therapeutic vaccine. Such vaccines are typically formulated as pharmaceutical compositions, e.g. sterile injectable compositions.

In some embodiments, the expression vector can be a bacterial vector that can be expressed in a genetically-engineered bacterium. Alternatively or additionally, the expression vector can be a yeast vector that can be expressed in yeast (e.g., Saccharomyces cerevisiae). These expression vectors can be useful for manufacturing the vaccines. Moreover, the recombinant nucleic acids described herein need not be limited to viral, yeast, or bacterial expression vectors. Suitable vectors also include DNA vaccine vectors, linearized DNA, and mRNA, all of which can be transfected into suitable cells following protocols well known in the art.

Further embodiments concern kits for use with methods and compositions described herein. Compositions and nucleic acid vaccines may be provided in the kit. The kits may also comprise bioinformatics tools (e.g., for the rapid assisted genetic design of the vaccines described herein), and/or can include a suitable container, nucleic acid vaccine compositions detailed herein and optionally one or more additional agents such as other antiviral agents, anti-fungal, anti-bacterial and/or anti-parasite agents. In some embodiments, the kit can include the nucleic acids, the modified coronavirus spike protein or fragment thereof, the protein complex, the vaccine, the pharmaceutical composition, and/or the liposome nanoparticle as described herein and/or an instruction of how to use the kit.

### EXAMPLES

The invention is further described in the following examples, which do not limit the scope of the invention described in the claims.

### Example 1. Optimization of mRNA Sequence

Sequence optimization of SARS-CoV-2 S protein coding region (SEQ ID NO: 1) was performed. The optimization included, for example, adjustment of the codon preference for expression in human, adjustment of the usage frequency of commonly used codons, and increase of the sequence GC content. The optimized nucleotide sequence is shown in SEQ ID NO: 2, which can make the structure of the transcribed mRNA more stable, and can also increase the target protein translation efficiency in mammals and humans.

### Example 2. Expression of SARS-CoV-2 S Trimer Protein in Pre-Fusion Stable Form

The S protein is composed of an extracellular domain (ectodomain, ECD), a transmembrane domain (TM) and a C-terminal cytoplasmic region (CP). The extracellular domain can be further divided into a secretion signal peptide (SP), an N-terminal domain (NTD), a receptor binding domain (RBD), an intrinsic membrane fusion peptide domain (FP) and two heptad repeats (HR1 and HR2). The S protein is categorized as a Class I viral fusion protein. A schematic structure of the S protein is shown in FIGS. 1A-1B. The pre-fusion structure is the functional conformation of the S protein. After fusion, a large number of sensitive neutralizing epitopes that only exist in the pre-fusion form are masked. Expression of a pre-fusion stable form of the SARS-CoV-2 S trimer protein is a key step to the development of a safe and effective SARS-CoV-2 vaccine. The following SARS-CoV-2 S trimer proteins were expressed with a pre-fusion stable form:

(1) The coding region encoded the extracellular domain ECD of S protein (amino acids 1-1213). Single or double proline mutations were also introduced in a turn between the central helix (CH) and heptad repeat 1 (HR1), e.g., K986P and/or V987P. The double proline mutated sequence was selected. The substitution of the two proline residues significantly improves the stability of the pre-fusion conformation.

(2) Because the S2 subunit can be transformed into the post-fusion conformation by inserting the fusion peptide domain (FP) into the host cell membrane. The fusion peptide FP domain (amino acids 788-806) was removed from the S proteins in (1), to further improve the stability of the pre-fusion conformation.

(3) The coding region encoded a membrane-anchored S protein (amino acids 1-1237), of which the C-terminal cytoplasmic region (CP; amino acids 1238-1273) was removed. SEQ ID NO: 3-8 are related to the extracellular domain ECD of S protein. The transmembrane domain (TM) were then added to the extracellular domain ECD of S protein. In addition, single or double proline mutations were also introduced, e.g., K986P and/or V987P.

(4) The fusion peptide domain FP (amino acids 788-806) was removed from the S proteins in (3).

(5) In order to prevent SARS-CoV-2 S protein from being cleaved by Furin-like proteases or lysosomal proteases during the intracellular packaging process, in which 51 and S2 are secreted in non-fusion conformation, amino acid sequence of the cleavage site in the S proteins in (1)-(4) were mutated. For example, the S1/S2 cleavage site residues RRAR (amino acids 682-685) were mutated to GGSG.

(6) The S2 cleavage site residues KR (amino acids 814 and 815) were mutated to AN in the S proteins in (5). Mutation of the S1/S2 cleavage site and the S2 cleavage site further improves the stability of the S protein.

(7) The secretion signal peptide (amino acids 1-13) can be replaced with an immunoglobulin heavy chain variable region (IGVH) signal sequence in the S proteins in (1)-(6). Sequence of the IGVH signal sequence is shown in SEQ ID NO: 39.

(8) The C-terminus of the S proteins in (1)-(6) were fused with a trimer domain through a linker sequence to form trimer S proteins. For example, the linker sequence SAIG is fused with the T4 phage fibritin trimer motif (SEQ ID NO: 40) to form the trimer S proteins.

The above SARS-CoV-2 S trimer proteins in a pre-fusion stable form were expressed. Amino acid sequences of these proteins are set forth in SEQ ID NOs: 3-38, SEQ ID NO: 56, SEQ ID NO: 57, SEQ ID NO: 58, and SEQ ID NO: 59.

### Example 3. Preparation of mRNA

The mRNA encoding the SARS-CoV-2 S protein in a pre-fusion stable form was prepared by in vitro transcription. The nucleic acid sequence of the S protein coding region was obtained by synthesis. The nucleic acid sequence of the coding region was connected with the plasmid vector by molecular cloning methods, to obtain the DNA plasmid template for in vitro transcription.

TABLE 1
Transcription starting site

| Transcribed | | | |
|---|---|---|---|
| sequence | _{PPP}GGG | _{PPP}CGG | _{PPP}AGG |
| T7 promoter | 5′-TAATACGACTCA | 5′-TAATACGACTCA | 5′-TAATACGACTCA |
| sense | CTATAGGG-3′ | CTATACGG-3′ | CTATAAGG-3′ |
| sequence | (SEQ ID NO: 60) | (SEQ ID NO: 61) | (SEQ ID NO: 62) |
| T7 promoter | 3′-ATTATGCTGAGT | 3′-ATTATGCTGAGT | 3′-ATTATGCTGAGT |
| antisense | GATATCCC-5′ | GATATGCC-5′ | GATATTCC-5′ |
| sequence | (SEQ ID NO: 63) | (SEQ ID NO: 64) | (SEQ ID NO: 65) |

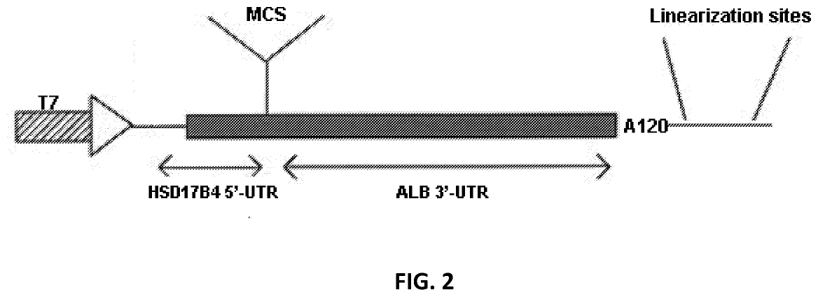

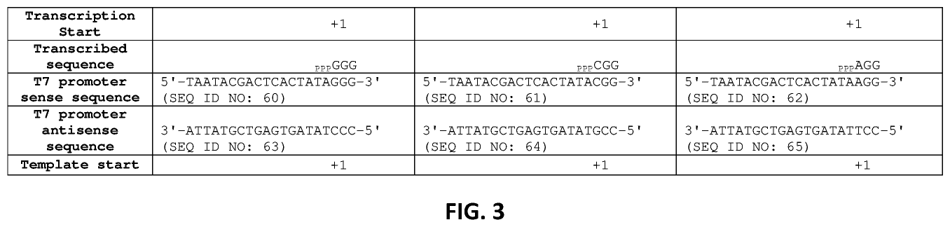

As shown in FIGS. 2, 3, the elements contained in the DNA plasmid template included the T7 promoter sequence (TAATACGACTCACTATA (SEQ ID NO: 66)), and the starting nucleic acid sequence can be selected from AGG, GGG, or CGG, as shown in the above table. Other elements included: the 5′-UTR sequence (SEQ ID NO: 41), the 3′-UTR sequence (SEQ ID NO: 43), the 3′ end polyA sequence (120 nucleotides), the XbaI digestion site (TCTAGA) following the 3′ end polyA sequence. The transcribed mRNA sequences are set forth in SEQ ID NOs: 44-48 (corresponding to amino acid sequences SEQ ID NOs: 56-59).

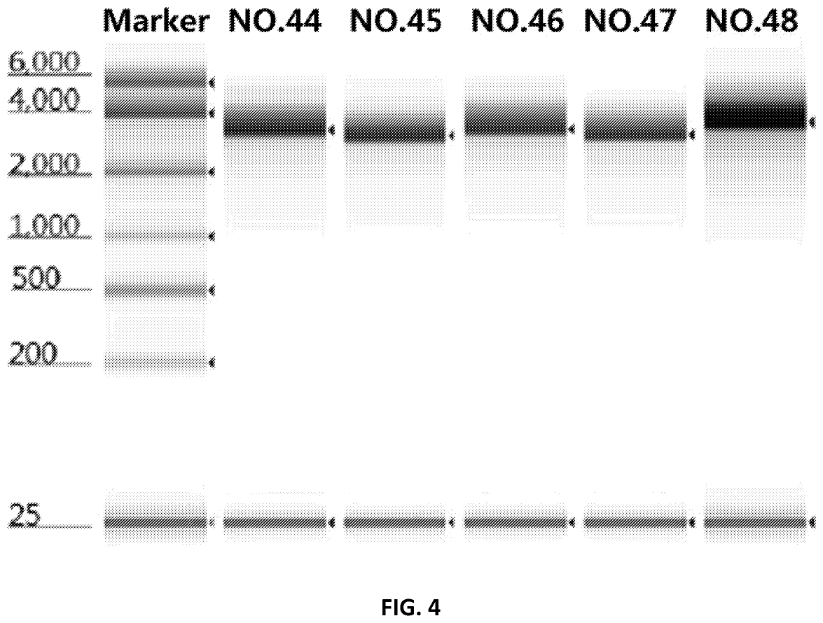

The mRNA size and integrity of SEQ ID NOs: 44-48 were analyzed by Agilent 2200 Tapestation automatic electrophoresis system. As shown in FIG. 4, each of the transcribed mRNAs showed a single band and no degradation was detected.

The experiment was performed as follows. First, the DNA plasmid template was linearized by XbaI digestion. The digestion system was mixed according to the following table.

TABLE 2

| RNase Free H₂O | Add to 500 μl | |
|---|---|---|
| 10× FastDigest Buffer | 50 | μl |
| FastDigest Xbal | 50 | μl |
| pDNA plasmid (500 ng/μL) | 200 | μl |

The reaction system was mixed with a vortex mixer, briefly centrifuged and then placed in a constant temperature mixer at 37° C./500 rpm for 2 hours. After mixing, 1 μL of the digested product was loaded into a 1% agarose gel for electrophoresis analysis. A single bright band was observed in the corresponding lane, indicating that the plasmid template was completely linearized. Afterwards, the linearized DNA plasmid template was recovered using phenol/chloroform/isoamyl alcohol (pH >7.8). The plasmid concentration and purity were verified using a spectrophotometer. The in vitro transcription was then performed using the linearized DNA template, according to the reaction system in the table below.

TABLE 3

| RNase-Free Water | Add to 1 ml | |
|---|---|---|
| T7 Reaction Buffer (5×) | 200 | μL |
| rNTP mix (100 mM) | 300 | μL |
| Cap analog (50 mM) | 200 | μL |
| Linearized DNA template | 50 | μg |
| T7 Enzyme Mix (200 U/μL) | 100 | μL |

The reaction system was placed in a constant temperature shaker at 37° C./300 rpm for 4 hours. After the reaction, 50 units of DNase I was added to the system to digest the plasmid DNA template at 37° C./300 rpm for 45 minutes. After the DNase digestion, lithium chloride (LiCl) was added to the reaction system to a final concentration of 2.5 M to precipitate RNA. The precipitated RNA was kept at −20° C. for at least 30 minutes. Afterwards, the reaction mixture was centrifuged at 12000 rpm for 20 minutes at 4° C. to pellet the precipitated RNA. The pellet was washed with 75% ethanol twice, and then centrifuged at 12000 rpm at 4° C. for 2 minutes. Supernatant was discarded and the pellet was air dried at room temperature. After the pellet was completely dried, RNase free water was used to dissolve the mRNA. The mRNA was further purified by high performance liquid chromatography (HPLC), and the size and integrity of the obtained mRNA were verified by Agilent 2200 Tapestation automatic electrophoresis system.

### Example 4. Expression and Detection of S Protein

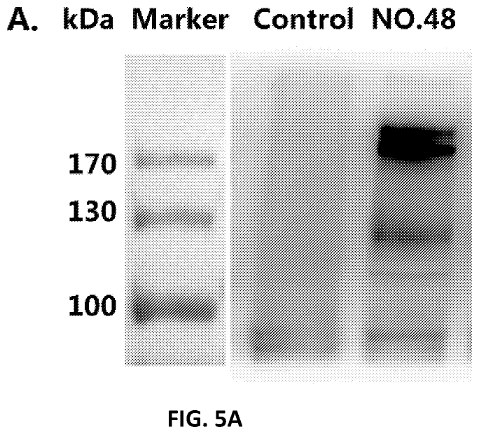

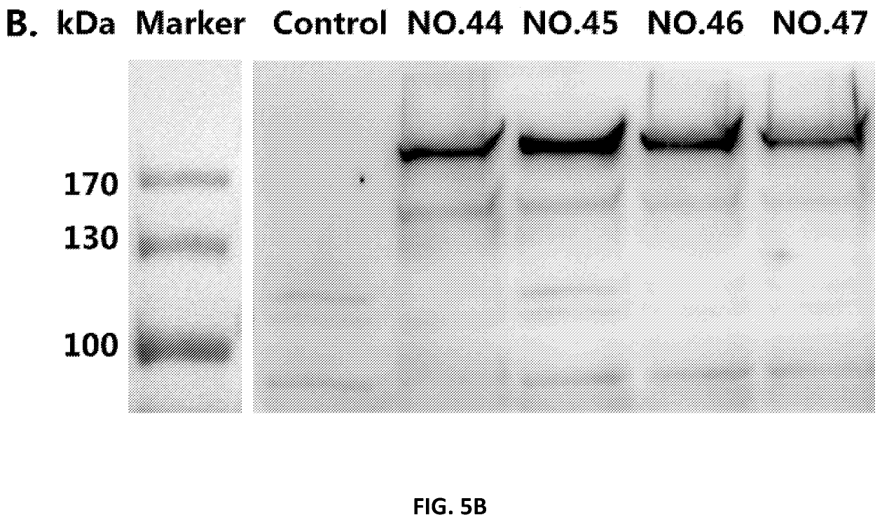

The purified mRNA (2.5 μg) encoding the pre-fusion stable form of S protein (SEQ ID NOs: 44-47 and wild-type S protein mRNA SEQ ID NO: 48) were used to transfect 293T cells. Untransfected cells were used as negative control. After 24 hours, S protein expression was verified by Western blot. As shown in FIG. 5A, the wild-type S protein was cleaved when expressed in cells. By contrast, the pre-fusion form of S proteins were more stable, and only one major protein band was detected (FIG. 5B).

The experiment was performed as follows. One day before transfection, 293T cells were seeded into a 6-well cell culture plate at 450,000 cells/well. 2.5 μg mRNA and 3.75 μl transfection reagent Lipofectamine MessagerMAX (ThermoFisher Scientific) were diluted with serum-free medium Opti-MEM (Gibco), respectively. Transfection was carried out according to the product manual. After the incubation, the prepared transfection complex was added to the cells and the plate was placed in a CO₂ incubator for 24 hours. Afterwards, the cells were collected, and the cells were lysed with RIPA lysis buffer for total protein extraction. 10μl of the extracted total protein was loaded into an SDS-PAGE gel. After the electrophoresis, the total protein in the SDS-PAGE gel was transferred to a membrane at constant voltage of 25 volts for 30 minutes. The membrane was analyzed by Western blot using an anti-SARS Coronavirus Spike 51 Subunit protein antibody (Sino Biological Inc.; Catalog number 40150-RP01), and a goat-anti-rabbit-HRP secondary antibody.

### Example 5. Immune Response of Different mRNA Sequences

The mRNA sequence encoding the pre-fusion stable S protein was prepared according to the methods in Example 3, and mRNAs with SEQ ID NOs: 44-47 were obtained. The mRNA lipid nanoparticle vaccines were prepared using the mRNAs, respectively.

The obtained vaccines were used in BALB/c mouse immunization test. The experiment was performed as follows. 6-8 week old female BALB/c mice (9 mice in each group) were administered twice with the vaccines on Day 0 and Day 14 by intramuscular injection. The injection dose was 10 μg, and the injection volume was 50 μL. After 7 days, the mouse spleens were isolated to separate splenic lymphocytes, and the splenic lymphocytes were then stimulated by S protein. The T lymphocytes secreting INFγ were detected by the ELISPOT (Enzyme-linked immune absorbent spot) method. The ELISPOT method can quantitatively measure the frequency of cytokine secretion for cells.

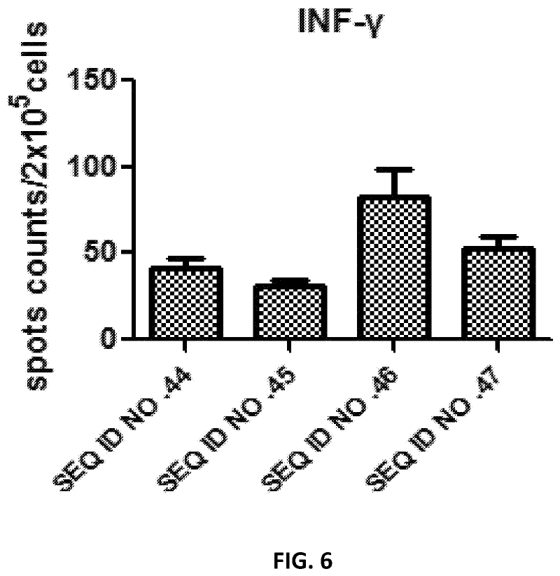

As shown in FIG. 6, SEQ ID NO.46 induced stronger cellular immune response in BALB/c mice. 14 days after the second immunization, the S protein specific IgG antibody was detected by indirect ELISA. The IgG antibody EC50 was calculated by fitting the antibody titer curves, as shown in the table below. The results showed that SEQ ID NO: 46 and SEQ ID NO: 45 induced higher titers of IgG antibodies in BALB/c mice

TABLE 4
EC50 titer of IgG antibody induced by mRNA
vaccines with different mRNA sequence

| | SEQ ID | SEQ ID | SEQ ID | SEQ ID |
|---|---|---|---|---|
| mRNA sequence | NO: 44 | NO: 45 | NO: 46 | NO: 47 |
| IgG EC50 titer | 18311 | 25210 | 85357 | 6822 |

### Example 6. Immune Response of Different Chemically Modified mRNA Vaccines

In order to optimize the mRNA so that it can have more stable structure and induce a higher level of immune response, mRNAs with chemically-modified nucleoside were prepared. The modified mRNAs (mRNA sequence shown in SEQ ID NO: 45) were packaged into liposome nanoparticles to generate LNP vaccines. SEQ ID NO: 45 was selected because SEQ ID NO: 45 provided a relatively higher IgG EC50 titer.

The chemical modifications are shown in the table below.

TABLE 5

| Properties | Modification |
|---|---|
| Modification 1 | pseudo-UTP |
| Modification 2 | 5-Me-CTP; Pseudo-UTP |
| Modification 3 | rUTP:1-N-Me-Pseudo-UTP = 1:1 |

In Modification 1, pseudo-UTP (instead of rUTP) was used during mRNA synthesis. In Modification 2, 5-Me-CTP and Pseudo-UTP (instead of rCTP and rUTP) were used during mRNA synthesis. In Modification 3, rUTP and 1-N-Me-Pseudo-UTP with a ratio of 1:1 were used during mRNA synthesis.

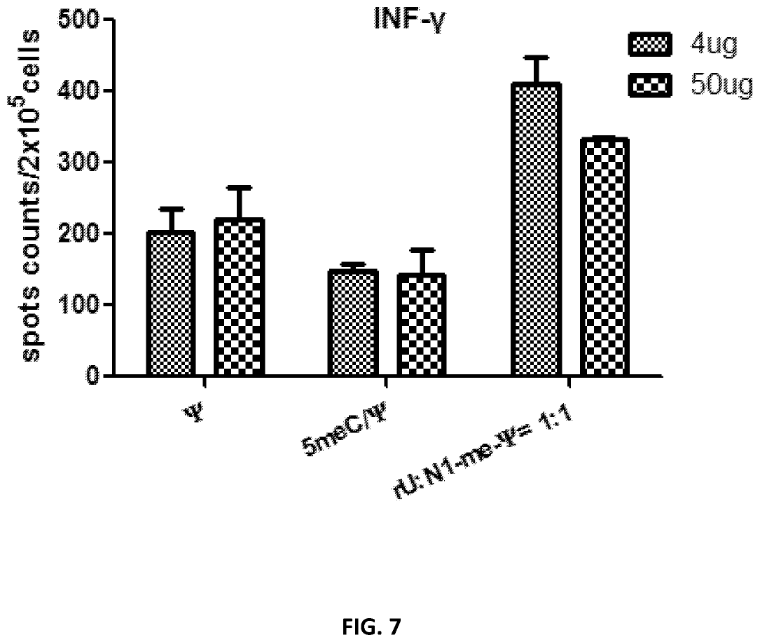

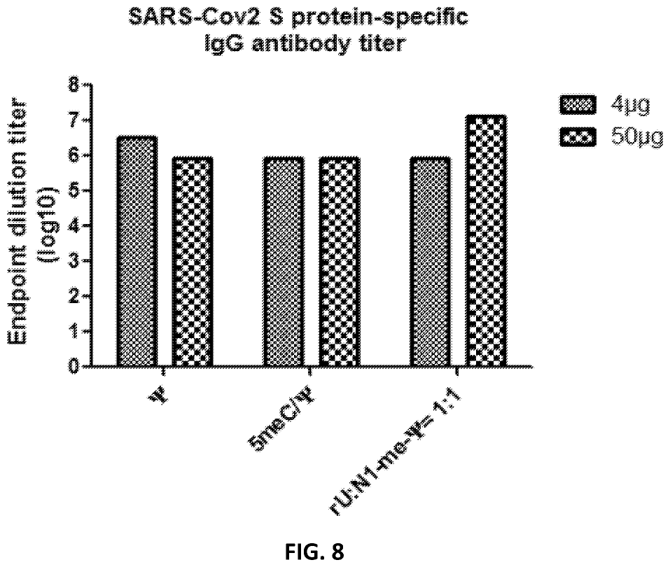

The three vaccines were used in BALB/c mouse immunization test. The experiment was performed as follows. 6-8 week old female BALB/c mice (9 mice in each group) were administered with the three vaccines by intramuscular injection, with an injection volume of 50 μl. The initial and booster immunizations were carried out on Day 0 and Day 14, respectively, and the immunization doses of 4 μg and 50 μg for Modification 1, Modification 2 and Modification 3 were tested. Seven days after the initial immunization, the mouse spleen was isolated to separate the splenic lymphocytes, and the splenic lymphocytes were then stimulated by S protein. The T lymphocytes secreting INFγ were detected by the ELISPOT method twice. As shown in FIG. 7, the spot count results showed that the mRNA vaccines with Modification 1, 2 and 3 induced stronger cell-mediated immune response in mice. 14 days after the booster immunization, the S protein specific IgG antibody titer was detected by indirect ELISA, and the results are shown in FIG. 8. All vaccines induced high IgG antibody titer in mice. The specific procedures of the ELISPOT test and the indirect ELISA test were carried out similar to the procedures in Example 5.

### Example 7. S Protein mRNA Vaccine Induces High Titer IgG Antibodies

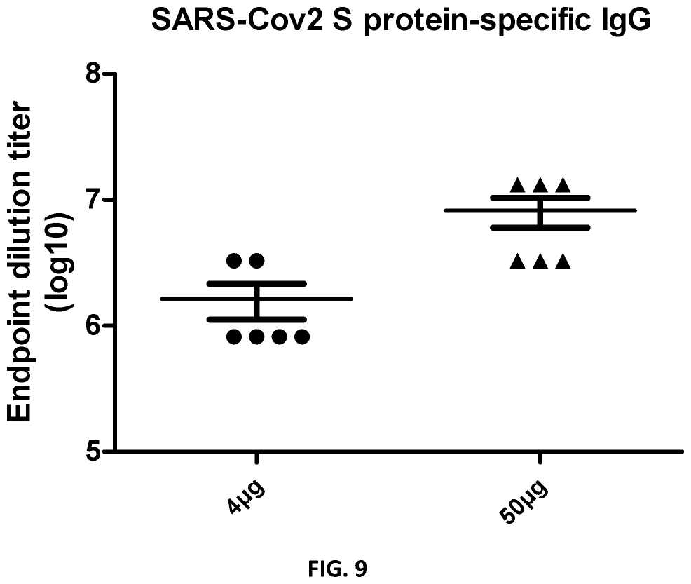

mRNA with Modification 1 in Example 6 was used to prepare a SARS-CoV-2 mRNA vaccine (mRNA sequence shown in SEQ ID NO: 45). The concentration of the prepared vaccine preparation was used for immunizing BALB/c mouse. BALB/c mice (6 mice/group) were vaccinated with the mRNA vaccine by intramuscular injection on Day 0 and Day 14. The doses were 4 μg and 50 μg, and the administration volume was 50 μL. The negative control was an equal volume of PBS solution. Mouse serum was collected after 28 days of booster immunization, and ELISA was used to detect the level of specific IgG antibodies produced in the mice. As shown in FIG. 9, the IgG antibody produced in mice increased in a dose-dependent manner, and the IgG titer of the high-dose group (50 μg) could reach 1.6×10⁷.

### Example 8. Persistent Effects of S Protein mRNA Vaccine

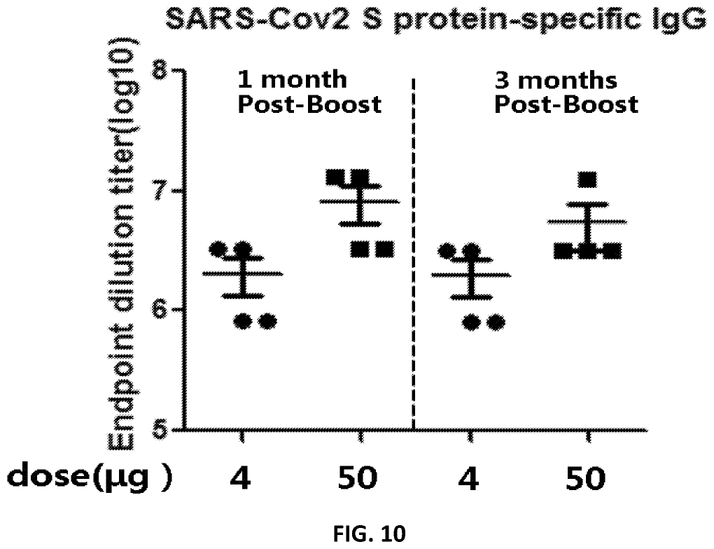

mRNA with Modification 1 in Example 6 was used to prepare a SARS-CoV-2 mRNA vaccine (mRNA sequence shown in SEQ ID NO: 45). The concentration of the prepared vaccine was determined and used for immunizing BALB/c mouse. BALB/c mice (4 mice/group) were administered with the mRNA vaccine by intramuscular injection on Day 0 and Day 14, respectively. The doses were 4 μg and 50 μg, and the administration volume was 50 μL. The negative control was an equal volume of PBS solution. Mice serum was collected 1 month and 3 months after the booster immunization, and the level of specific IgG antibodies produced in the mice was detected by ELISA. As shown in FIG. 10, 3 months after the booster immunization, there was no decrease in SARS-Cov2 S protein specific IgG antibody titer in mice.

### Example 9. Production of High Titer IgG Antibodies by Immunizing Difference Mouse Strains

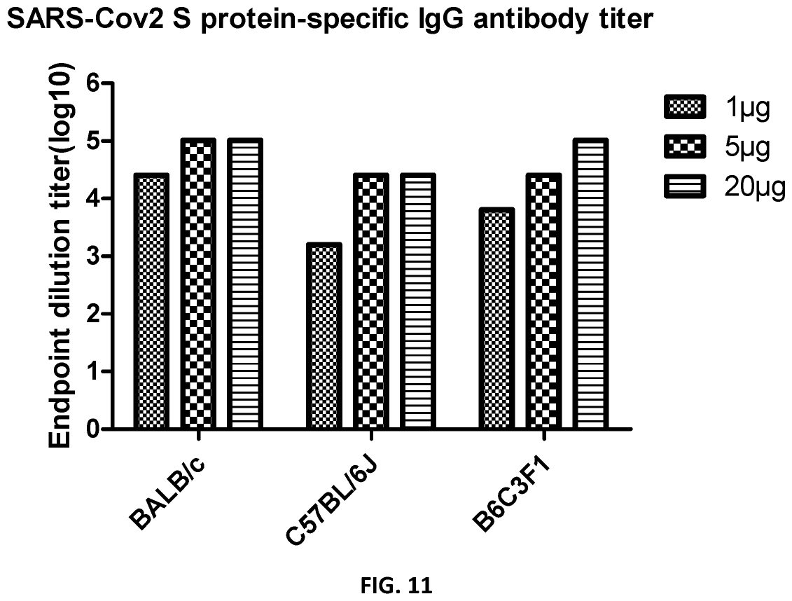

mRNA with Modification 1 in Example 6 was used to prepare a SARS-CoV-2 mRNA vaccine (mRNA sequence shown in SEQ ID NO: 45). The concentration of the prepared vaccine was determined and used for immunizing BALB/c mice, C57BL/6J mice and B6C3F1 mice. The three different mouse strains (9 mice/group) were given a single dose of the mRNA vaccine by intramuscular injection. The doses were 1 μg, 5 μg and 20 μg. The administration volume was 50 μL. The negative control was an equal volume of PBS solution. Mouse serum was collected 14 days after vaccination. The serums collected from the 9 mice in each group were mixed, and specific IgG antibodies produced in the mice were detected by ELISA. As shown in FIG. 11, a single vaccination of SARS-CoV-2 mRNA vaccine produced specific IgG antibodies with high titers. Different titers in IgG antibodies were detected in different mouse strains. Compared with BALB/c and B6C3F1 mice, IgG antibody titers in C57BL/6J mice were slightly lower. The antibody titer produced by a single vaccination of high-dose mRNA vaccine in BALB/c and B6C3F1 mice reached about 1×10⁵.

### Example 10. S Protein mRNA Vaccine Induces Neutralizing Antibodies

mRNA with Modification 1 in Example 6 was used to prepare a SARS-CoV-2 mRNA vaccine (mRNA sequence shown in SEQ ID NO: 45). The concentration of the prepared vaccine was determined and used for immunizing BALB/c mice. BALB/c mice (4 mice/group) were administered with the mRNA vaccine by intramuscular injection on Day 0 and Day 14, respectively. The doses were 4 μg and 50 μg, and the administration volume was 50 μL. The negative control was an equal volume of PBS solution. Mouse serum was collected 28 days after the booster immunization, and the S protein-specific neutralizing antibody was detected by pseudovirus neutralization test. The neutralizing antibody test results are shown in the table below. Both low-dose (4 μg) and high-dose (50 μg) immunized mice produced neutralizing antibodies, and the high-dose group produced higher titers of neutralizing antibodies.

TABLE 6
Neutralizing antibody test results

| | | Neutralizing antibody |
|---|---|---|
| Group | Sample number | titer(IC50) |
| PBS control group | 1001 | <30 |
| | 1002 | <30 |
| | 1003 | <30 |
| | 1004 | 47 |
| Immunization group-low dose | 2001 | 1230 |
| | 2002 | 398 |
| | 2003 | 54 |
| | 2004 | 128 |
| Immunization group-high dose | 3001 | 3090 |
| | 3002 | 977 |
| | 3003 | 3019 |
| | 3004 | 4677 |

The experiment was performed as follows. The serum was inactivated in a 56° C. water bath for 30 minutes, centrifuged at 6000 g for 3 minutes, and then supernatant was transferred to a 1.5 ml centrifuge tube. The serum was serially diluted with DMEM complete medium supplemented with penicillin and streptomycin. Pseudovirus was diluted to 1.3×10⁴ TCID50/ml with DMEM complete medium, and then added to each well at 50 μl/well, to make each well contain 650 TCID50 (median tissue culture infective dose). Then, 50 μl of diluted serum was added to each well, and the 50 μl of pseudovirus mixed with the 50 μl of serum (containing antibody) were incubated at 37° C. for 1 hour. After the incubation, 100 μl of cells were added to each well of a 96-well plate to make 2×10⁴ cells per well. For controls, pseudovirus control wells, serum toxicity control wells, and normal cell blank control wells were set aside. The 96-well plate was then placed into a cell culture incubator and incubated at 37° C. and 5% CO₂ for 20-28 hours. After 20-28 hours, the 96-well plate was taken out of the cell incubator. 150 μl of supernatant was discard by pipetting, and then 100 μl luciferase detection reagent was added to each well to detect luciferase chemiluminescence. The neutralization inhibition rate was calculated as: inhibition rate=[1−(average luminous intensity of the sample group−average luminous intensity of the blank control group)/(average luminous intensity of the virus control group−average luminous intensity of the blank control group)]×100%. According to the results of the neutralization inhibition rate, the Reed-Muench method was used to calculate the IC50 of the pseudovirus neutralization.

### Example 11. S Protein mRNA Vaccine Induces Th1 Immune Response

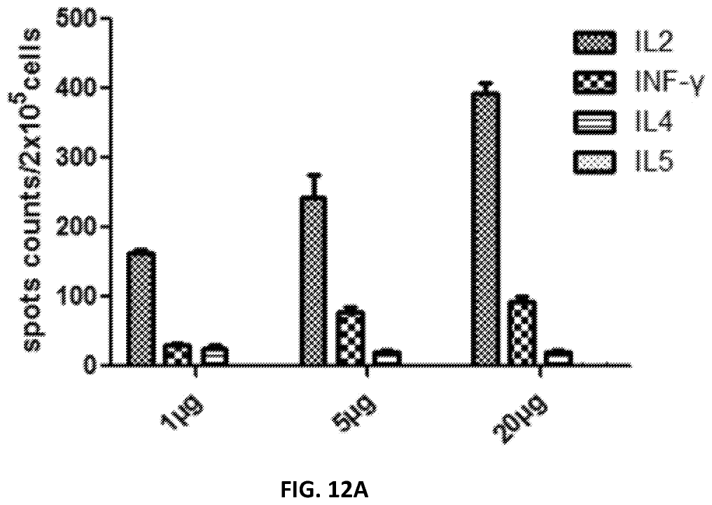

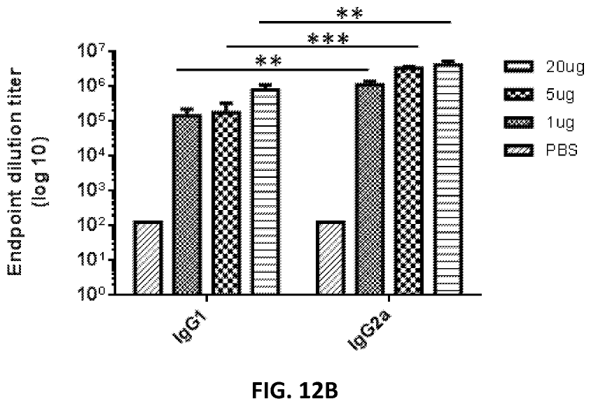

Th1/Th2 subgroups and their mutual balance play a key role in the regulation of immune response. It was reported that the Th2-type immune response induced by SARS vaccines was related to enhanced lung lesions after challenge studies, while Th1-type immune response had protective effects. mRNA with Modification 1 in Example 6 was used to prepare a SARS-CoV-2 mRNA vaccine (mRNA sequence shown in SEQ ID NO: 45). The concentration of the prepared vaccine preparation was determined and used for immunizing BALB/c mice. BALB/c mice (9 mice/group) were administered with the mRNA vaccine by intramuscular injection on Day 0 and Day 14, respectively. The doses were 1 μg, 5 μg and 20 μg, and the administration volume was 50 μL. The negative control was an equal volume of PBS solution. Seven days after the initial immunization, the mouse spleens were isolated to separate splenic lymphocytes. The splenic lymphocytes were then stimulated by S protein. Th1-type cytokines INFγ and IL2; Th2-type cytokines IL4 and IL5 were detected by the ELISPOT method. Detection of IL-5 and IL2 was carried out according to the manual of the Mabtech brand Mouse IL-5/IL2 ELISpotPLUS (HRP) kit. Detection of INFγ and IL4 was carried out according to the manual of the Dayou Mouse IFN-γ/IL4 ELISPOT kit. The results are shown in FIG. 12A.

The mouse serum was collected after 14 days of the booster immunization, and the titers of specific IgG1 and IgG2a antibodies produced in the mice were detected by ELISA. After vaccination, S protein antigen was used to stimulate spleen lymphocytes again. T lymphocytes secreting Th1-type cytokines INFγ and IL2 were significantly more than T lymphocytes secreting Th2-type cytokines IL4 and IL5.

The results of IgG antibody subtype detection are shown in FIG. 12B. The titers of IgG2a antibodies produced in mice were significantly higher than those of IgG1 antibodies. The cytokine release results and the IgG subtype determination showed that the immune response induced by the mRNA vaccine was more likely to be a Th1-type immune response.

### Example 12. Evaluate the Safety of mRNA Vaccines

Wistar rats including 4 males and 4 females, with a weight difference of no more than 10%, were selected and randomly divided into two groups: solvent control group and mRNA vaccine group. The concentration of the vaccine used in the measurement was 2 mg/mL.

Each animal was administered 3 times a day with the volume of each injection as 250 μl. The administration interval was 4 hours. Alternate administration to the left and right legs were performed. The total dosage level was 1.50 mg per rat, which was equivalent to 1200 times the maximum dosage level for human (assuming the maximum dosage level for human is 0.25 mg). Clinical observation was recorded as follows. Within 24 hours after administration, clinical observation was carried out every hour. Within 24-72 hours after administration, clinical observation was carried out once every 6 hours. Within 4-14 days after administration, clinical observation was carried out once a day. The symptoms of toxicity, the time when the symptoms appeared and disappeared, and the time of death (if occurred) were recorded. Body weight was recorded once a day after administration, and food intake was recorded every 2 days after administration. 14 days after the administration, all the remaining animals were weighed, then euthanized, and major organs were obtained by anatomy. Heart, liver, spleen, lung, kidney, thymus, lymph node, were weighted and relative organ weight (the organ weight/the subject weight×100%; also known as organ coefficient) was calculated. After anatomy, the heart, liver, spleen, lung, kidney, intestine, thymus, lymph node, muscle tissue at the injection site and other organs with pathological changes observed were stored in a fixation solution, and the pathology of each organ was examined by H&E staining. Lesion severity scores were given according to the table below.

TABLE 7
Lesion severity score standard

| | |
|---|---|
| Score = 0 | Under the conditions of the experiment, taking into |
| (does not | account the age, sex and strain of the animal, it can |
| exist) | be considered that the tissue is within the normal |
| | range and there is no pathological change. |
| Score = 1 | The first (lowest) grade of lesions in the 5 grades of |
| (minimal) | minimal, mild, moderate, severe and serious. |
| Score = 2 | The second grade of lesion degree in the 5 grades of |
| (mild) | minimal, mild, moderate, severe and serious. |
| Score = 3 | The third grade of lesion degree in the 5 grades of |
| (moderate) | minimal, mild, moderate, severe and serious. |
| Score = 4 | The fourth grade of lesion degree in the 5 grades of |
| (severe) | minimal, mild, moderate, severe and serious. |
| Score = 5 | The fifth (highest) grade of lesion degree in the 5 |
| (serious) | grades of minimal, mild, moderate, severe and serious. |

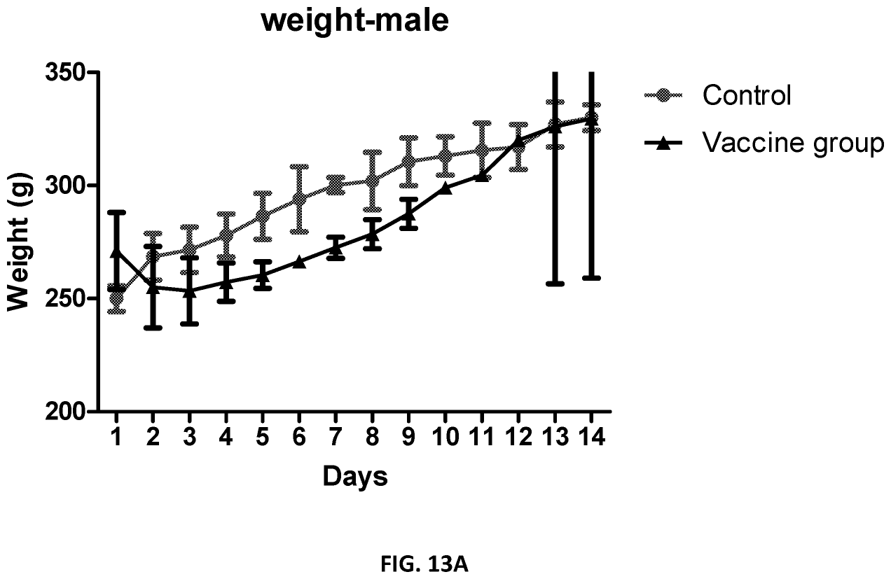

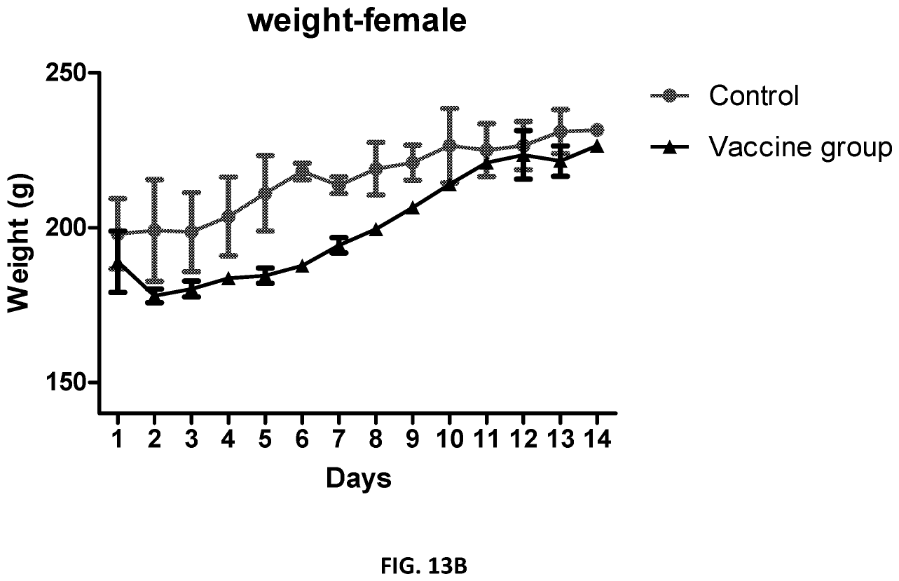

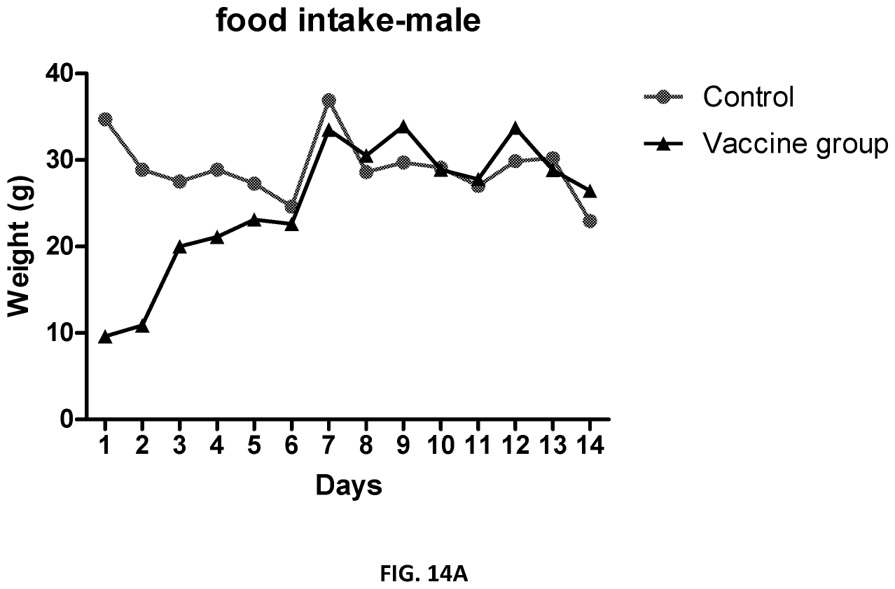

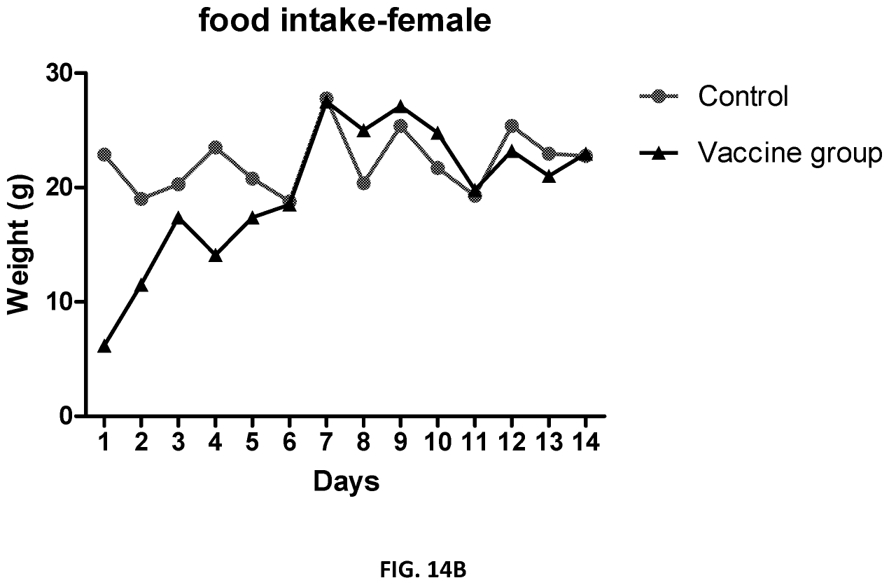

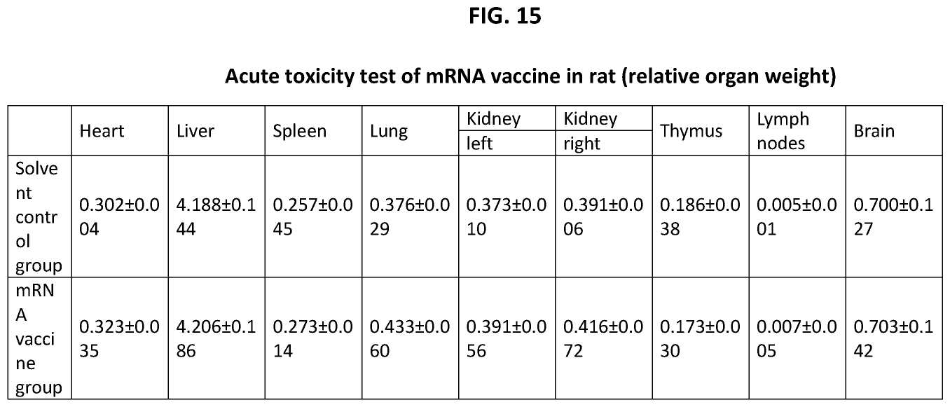

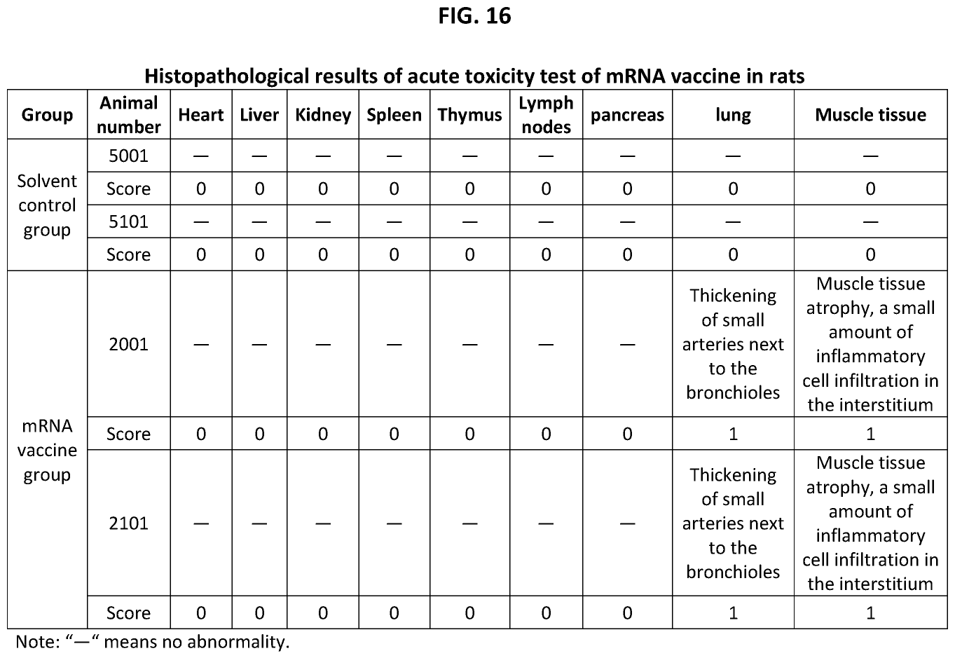

The weight change results are shown in FIGS. 13A-13B. Specifically, the body weight of male rats in the solvent control group continued to increase. The body weight of rats in the mRNA vaccine group dropped initially, then recovered to the pre-administration level on Day 7, and continued to increase subsequently. The results of changes in food intake are shown in FIGS. 14A-14B. The food intake per rat in the solvent control group within 24 hours was stable, within a range of 18-35 grams. The initial food intake of the rats in the mRNA vaccine group decreased initially, and returned to normal levels on Day 6-Day 7. The relative organ weight results are shown in FIG. 15. Compared with the solvent control group, the spleen and lymph node coefficients of rats in the mRNA vaccine group did not show significant difference. The results of histological changes are shown in FIG. 16. Compared with the rats in the solvent control group (one male and one male), the heart, liver, kidney, spleen, thymus, and lymph nodes of the rats in the mRNA vaccine group had no pathological changes. Based on the above results, the rats showed only slight pathological changes in the lungs and legs after the 1200 times higher dosage level of the mRNA vaccine.

### Example 13. Trimeric Structure of S Antigen Protein

The mRNA encoding the pre-fusion stable form of the S protein trimer with a FLAG tag and the mRNA encoding the wild-type S protein with a FLAG tag were prepared according to the method in Example 3. The riboFECT™ mRNA transfection Kit was then used to transfect HEK293T cells by reverse transfection.

The transfection was carried out as follows. First, 15 μL transfect reagent was added to 1 mL Opti-MEM medium and mixed. The mixed solution was kept at room temperature for 10 minutes. Afterwards, 10 μg of in vitro transcribed RNA was added and mixed, then kept at room temperature for 5 minutes. The mixture was then transferred to a 10 cm cell culture dish. Next, HEK293T cells were digested, counted, and then resuspended in fresh DMEM medium (containing 10% fetal bovine serum). A total of 5×10⁶ cells were added to the 10 cm cell culture dish pre-added with the transfection mixture, and mixed gently. The dish was then incubated at 37° C. overnight. On the next day, the culture medium in the dish was discarded and fresh medium was added. The cells were incubated for another 24 hours. After the incubation, protein samples were prepared in a non-denaturing manner. Specifically, after 48 hours of transfection, the transfected cells were centrifuged to discard the supernatant. The cell pellet was resuspended in 100 μL of a non-denaturing tissue/cell lysis buffer. After repeated pipetting, the cell suspension was kept on ice for 30 minutes, during which time the cell suspension was vortexed for 10 seconds every 10 minutes. Afterwards, the cell suspension was centrifuged at 12,000 g for 10 minutes at 4° C. Supernatant was collected and kept at −80° C.

Non-denaturing Western Blot detection of the S protein trimer was performed as follows. 10 μL of protein sample was mixed with 4 μL of non-denaturing protein loading buffer, and then loaded to a non-denaturing gel. Gel electrophoresis was carried out at a constant voltage of 120 volts for 2.5 hours. Afterwards, proteins within the gel were transferred to a PVDF membrane at a constant voltage of 25 volts for 2 hours. After the membrane transfer step, the PVDF membrane was washed with TBS buffer, and then blocked with a blocking solution (TBS containing 5% BSA) for 1 hour. After the blocking step, the membrane was incubated with 1:2500 diluted anti-DDDDK Tag antibody (Abcam plc.; Catalog number: Ab1162) overnight. On the next day, the membrane was washed three times (5 minutes each) with TBST buffer (TBS containing 0.05% Tween-20) on a horizontal shaker. The membrane was then incubated with goat anti-rabbit-HRP secondary antibody at room temperature for 1 hour. After incubation, the membrane was washed three times (5 minutes each) with TBST buffer. Next, the membrane was developed and analyzed in an automatic chemiluminescence image analyzer.

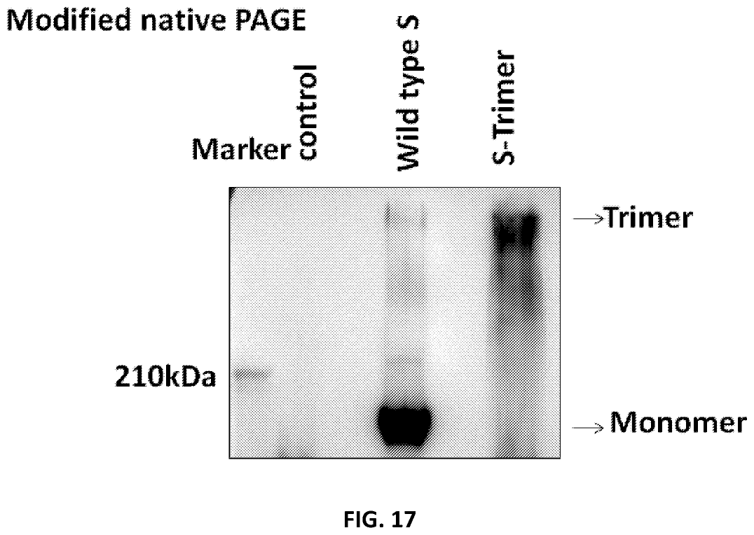

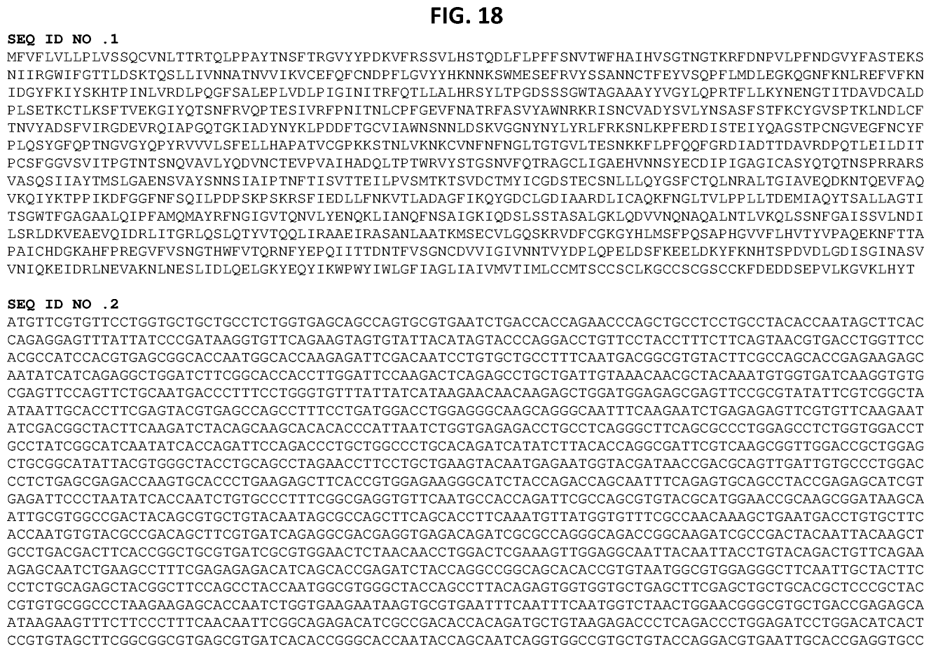

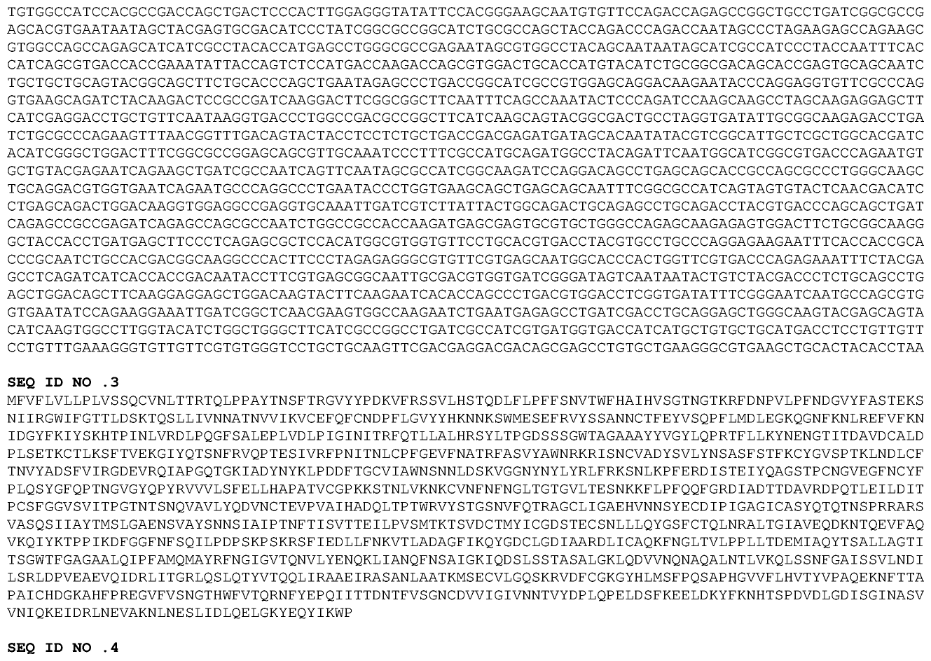

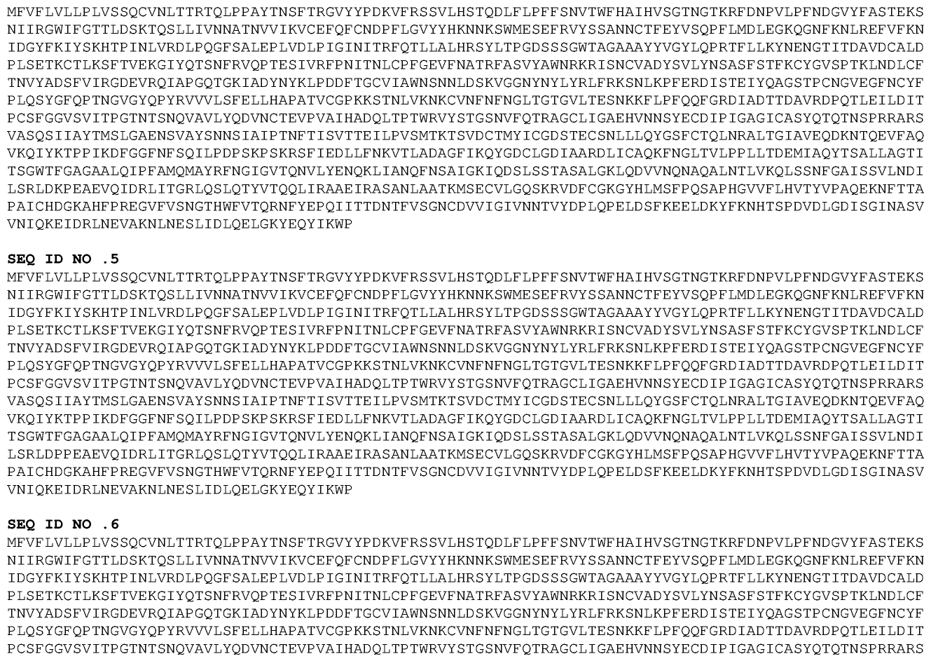

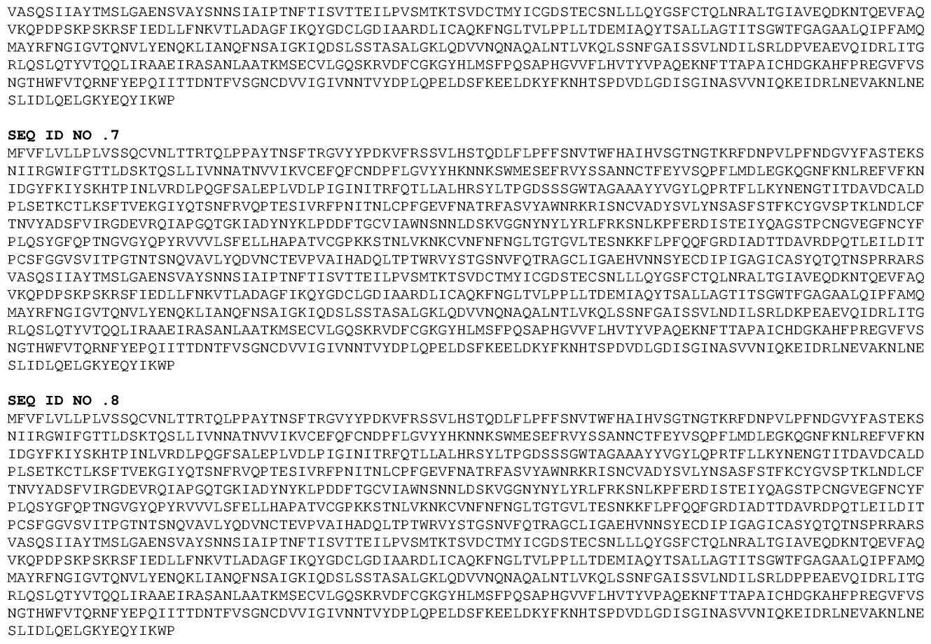

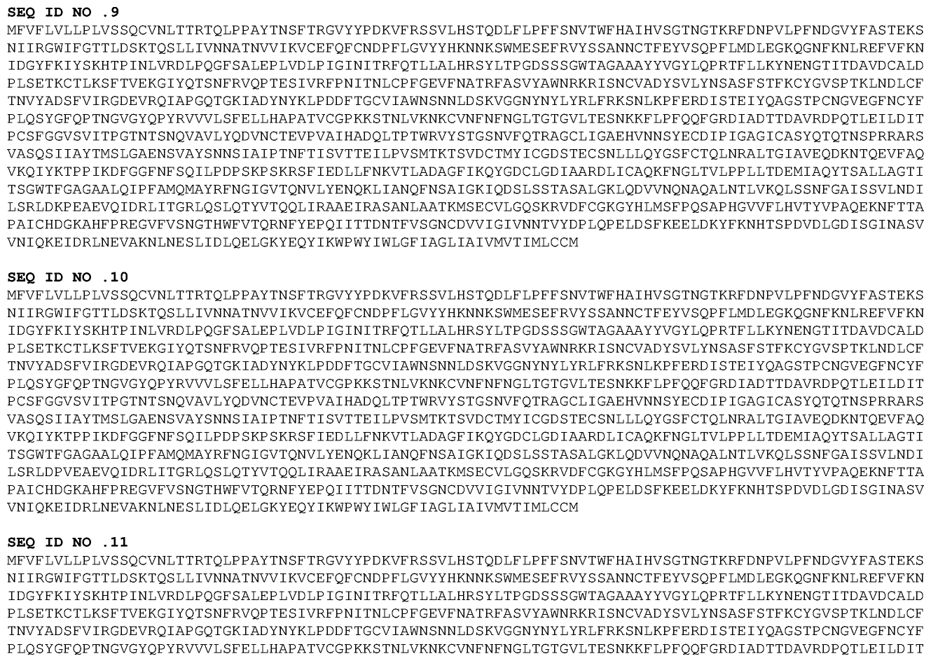

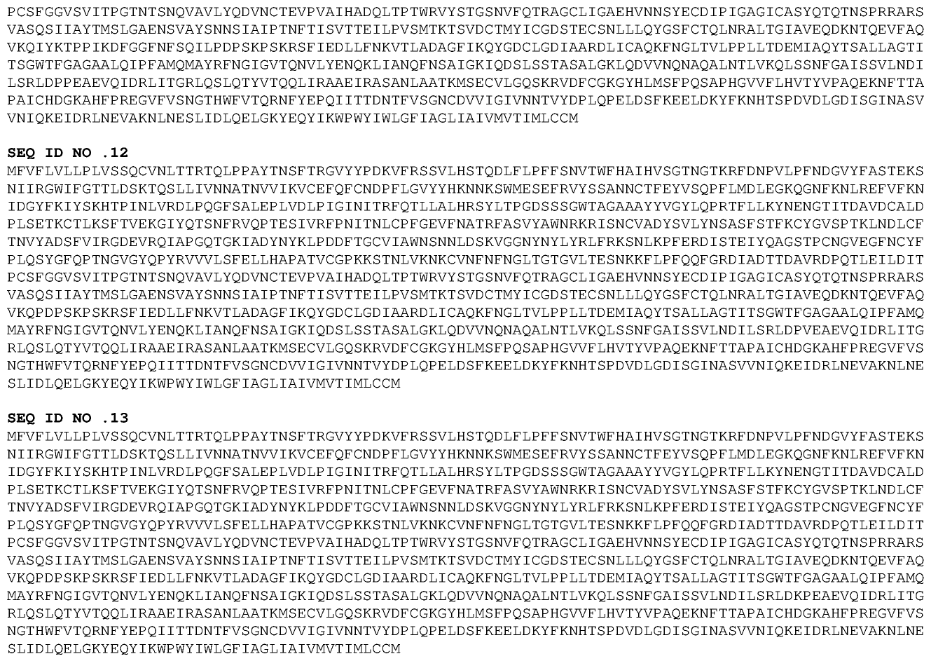

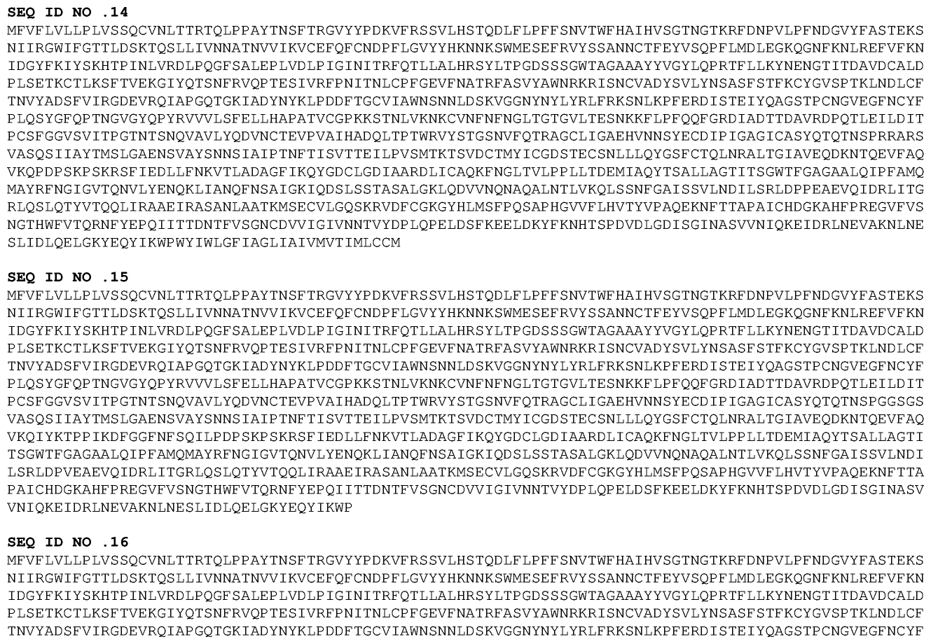

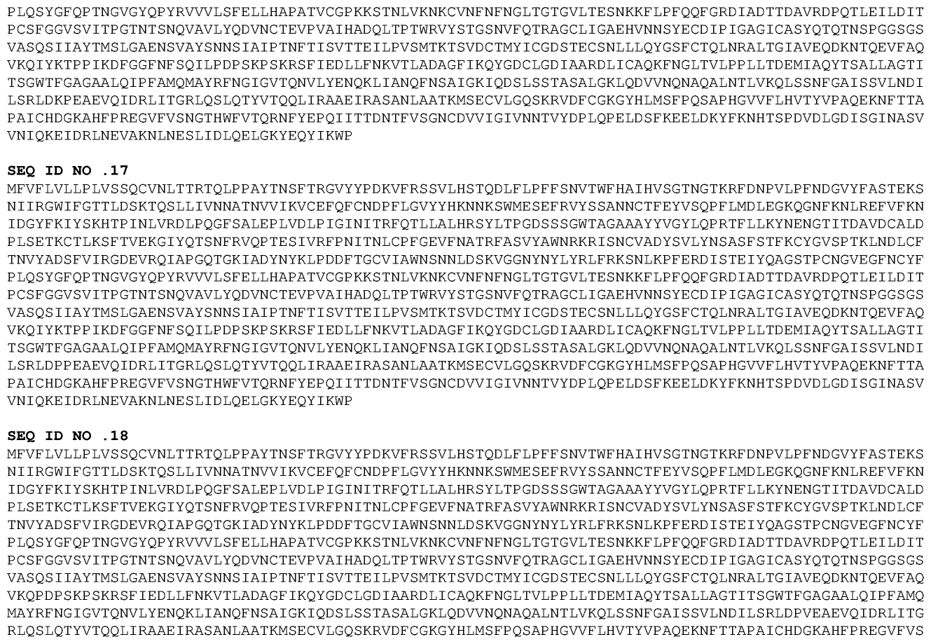

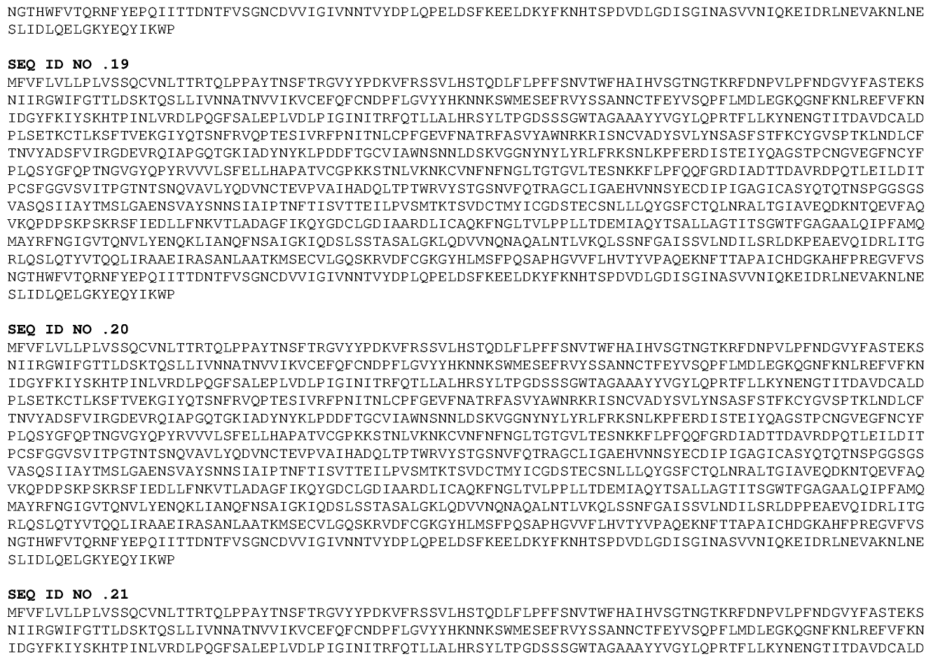

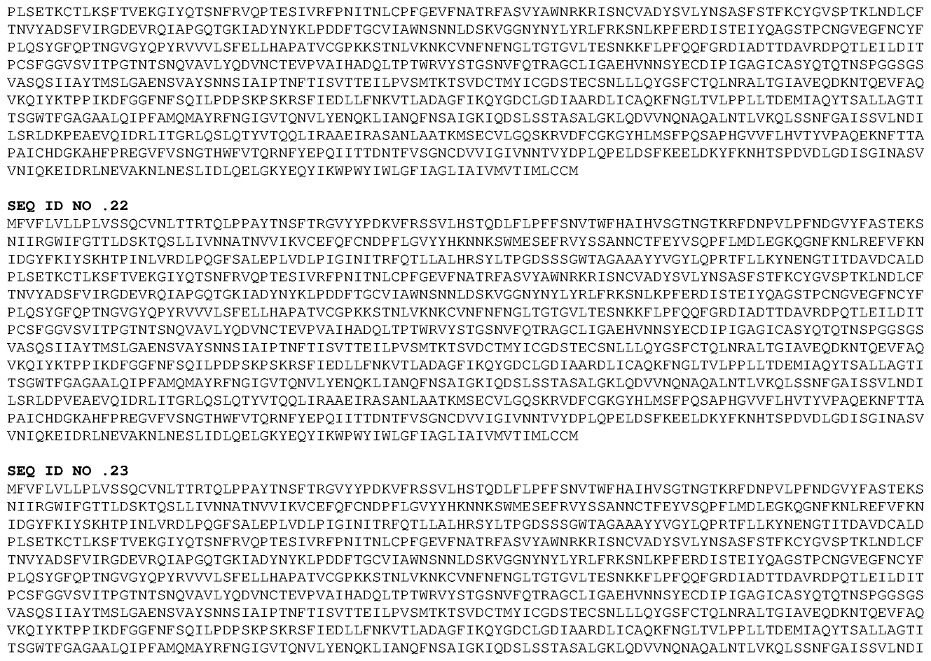

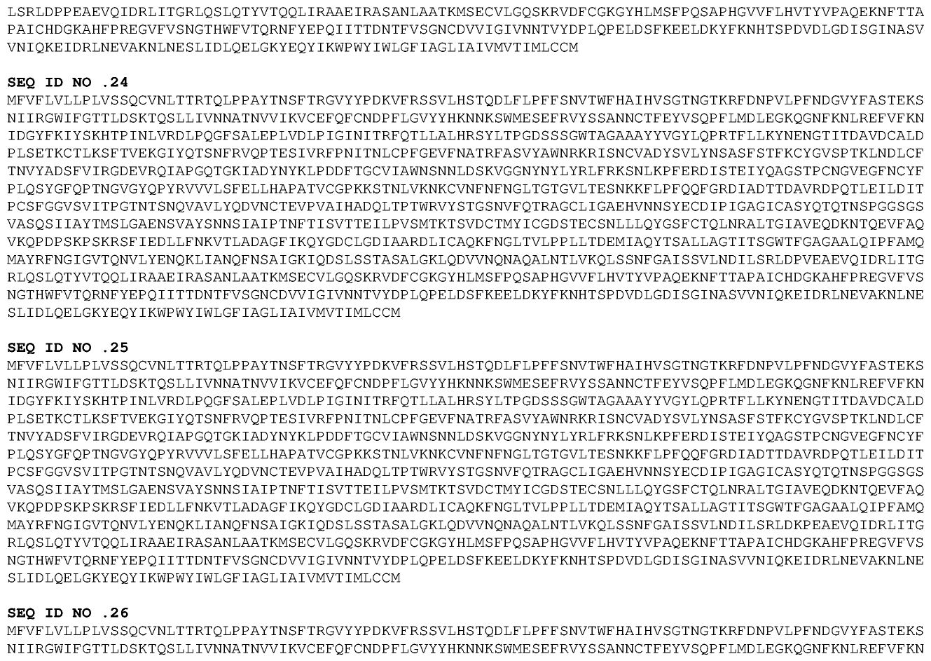

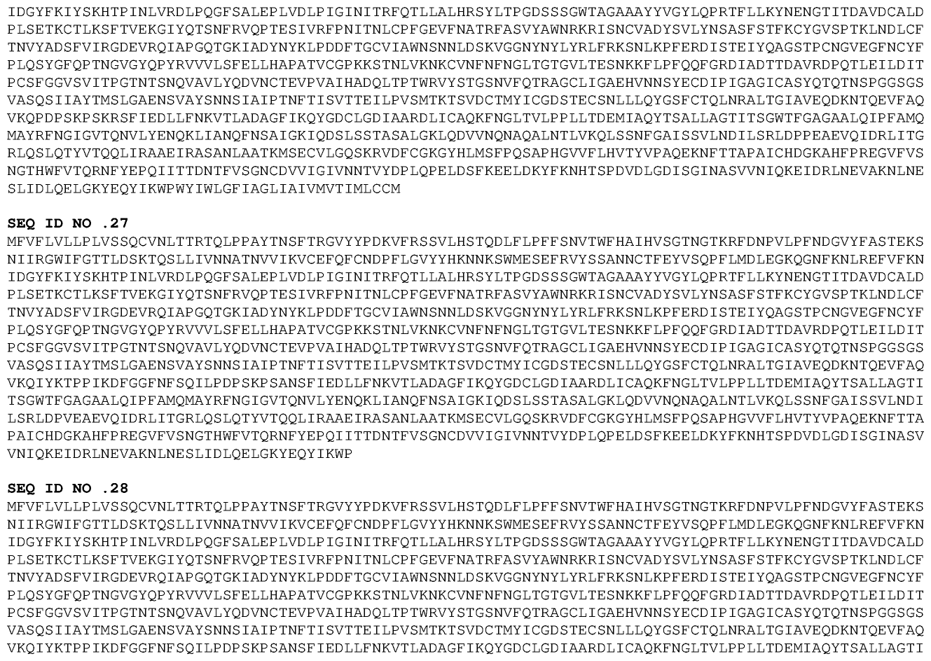

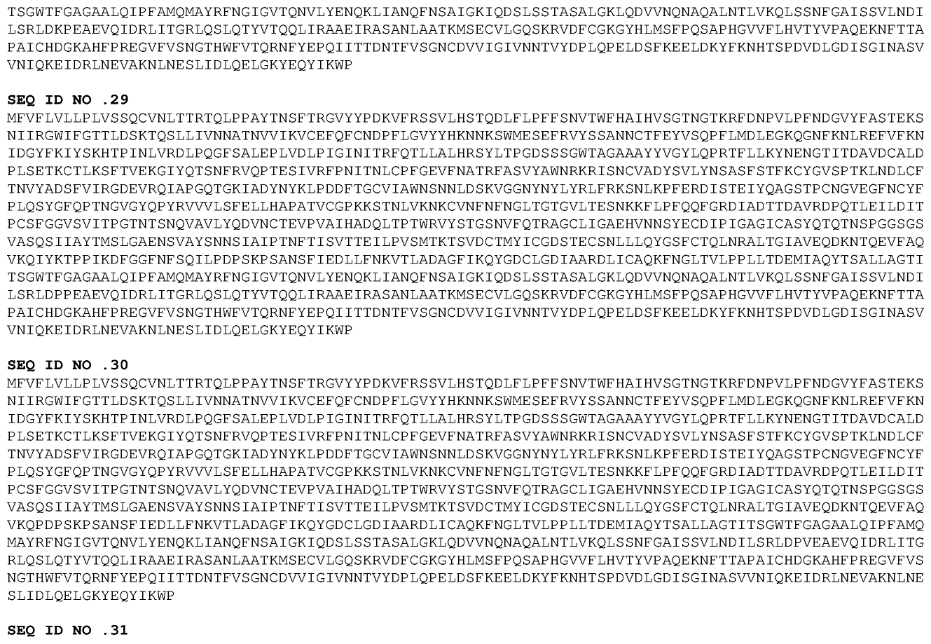

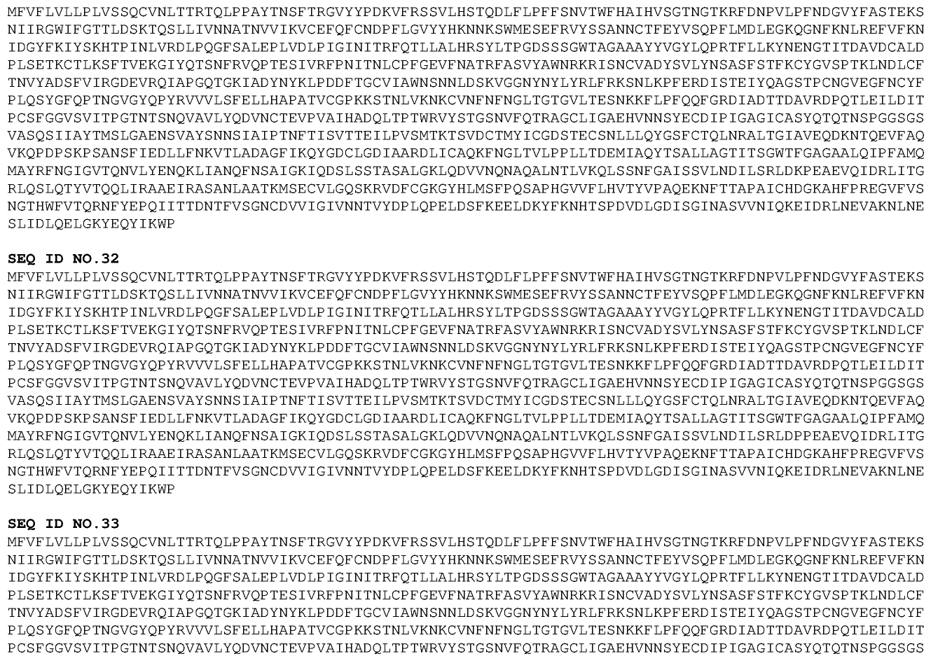

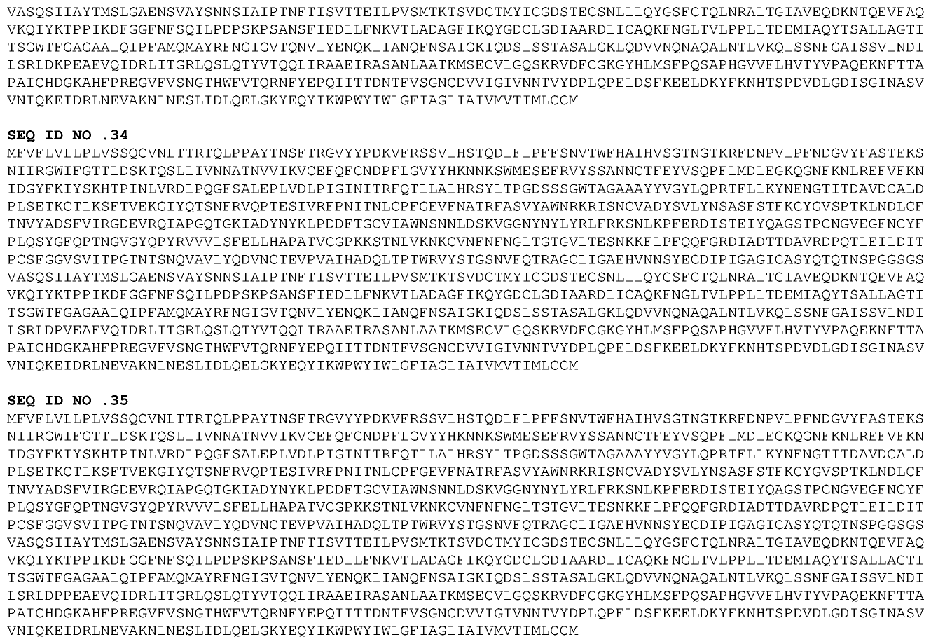

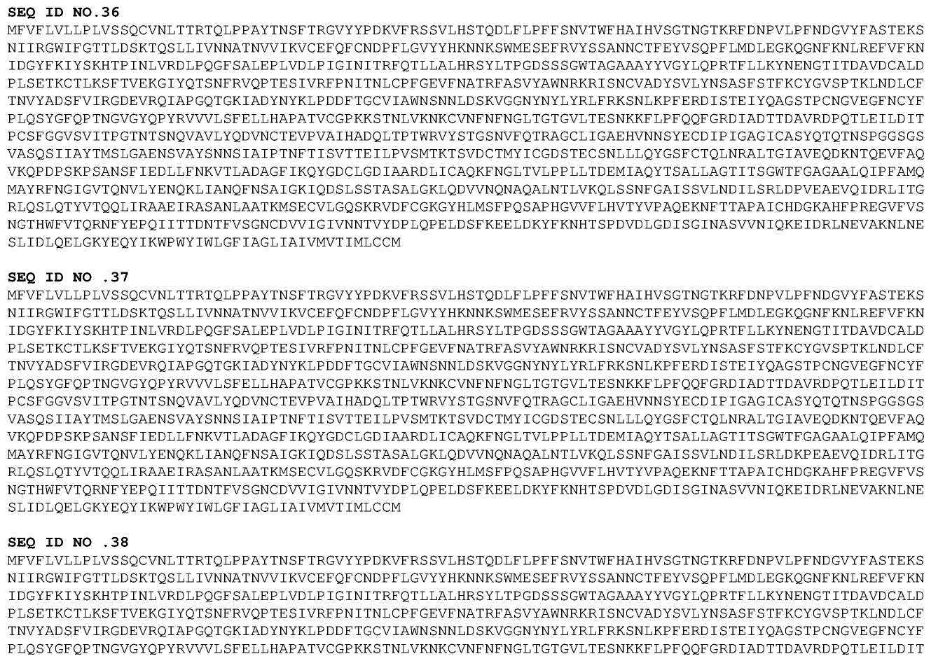

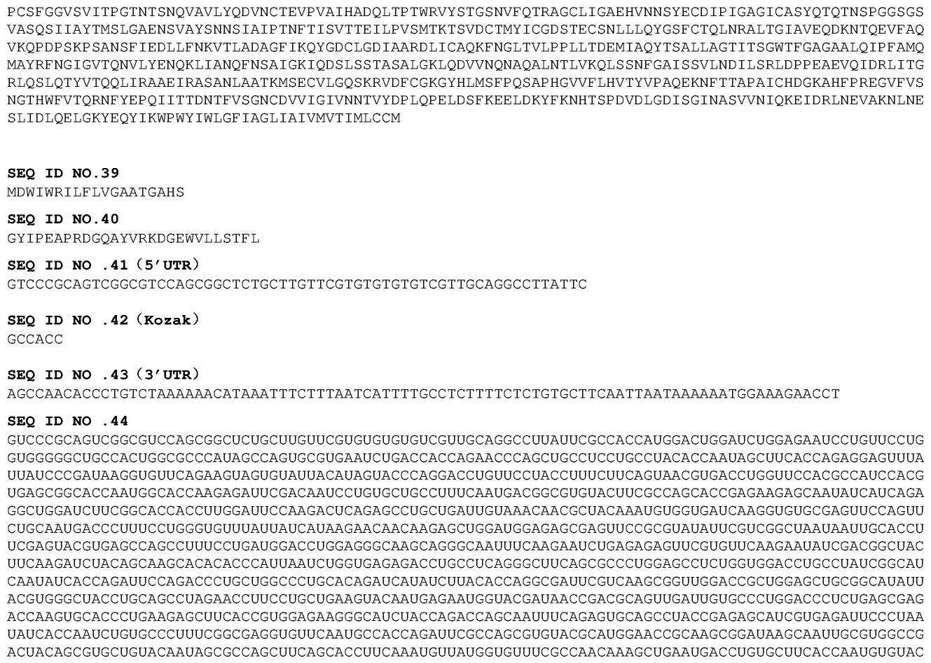

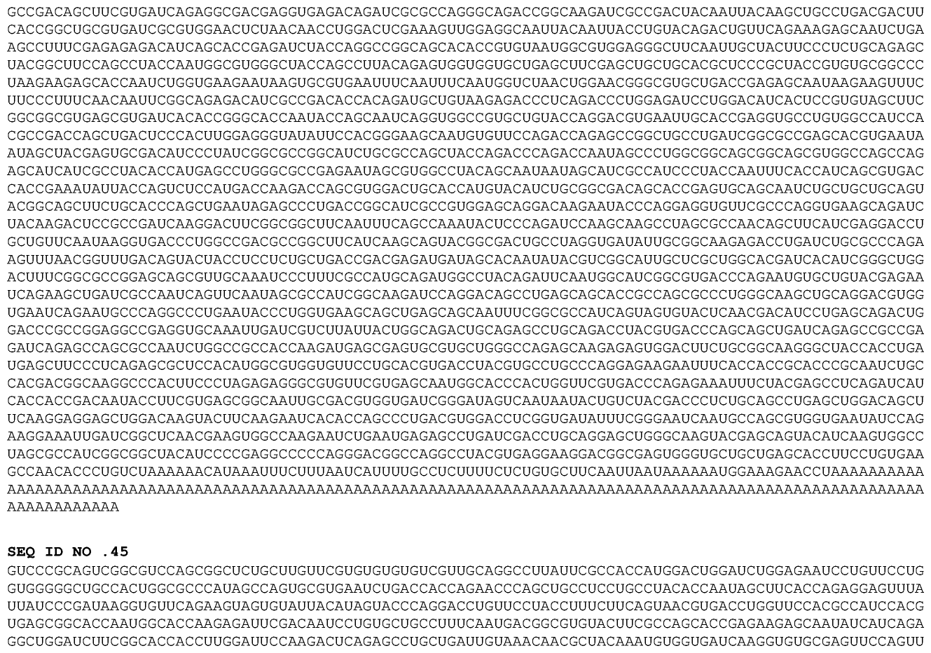

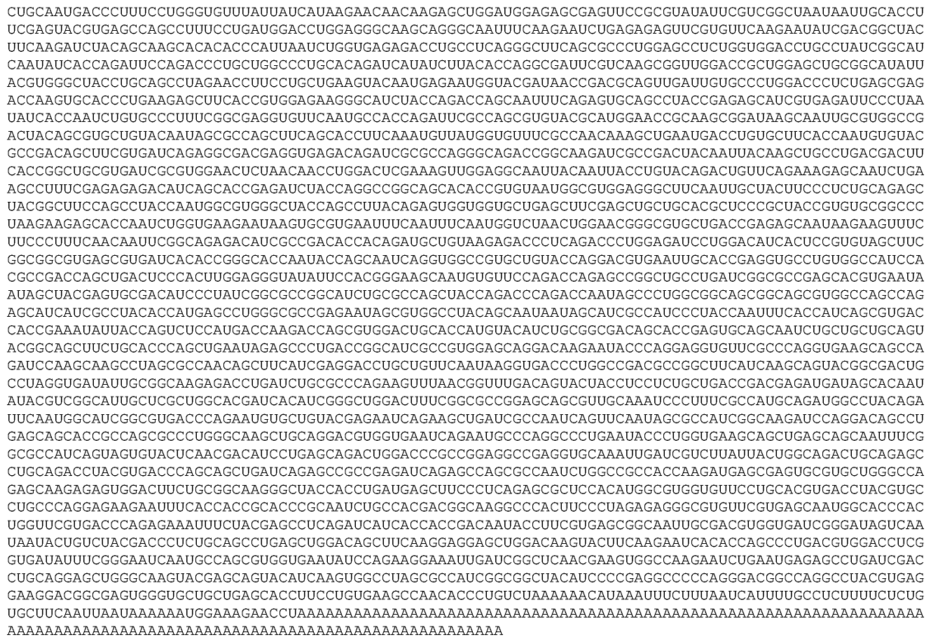

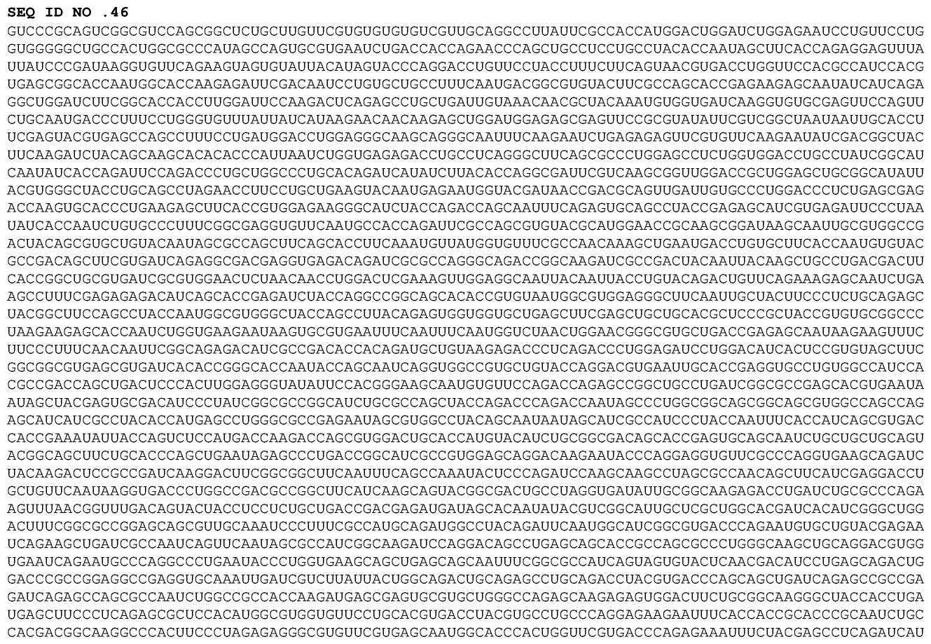

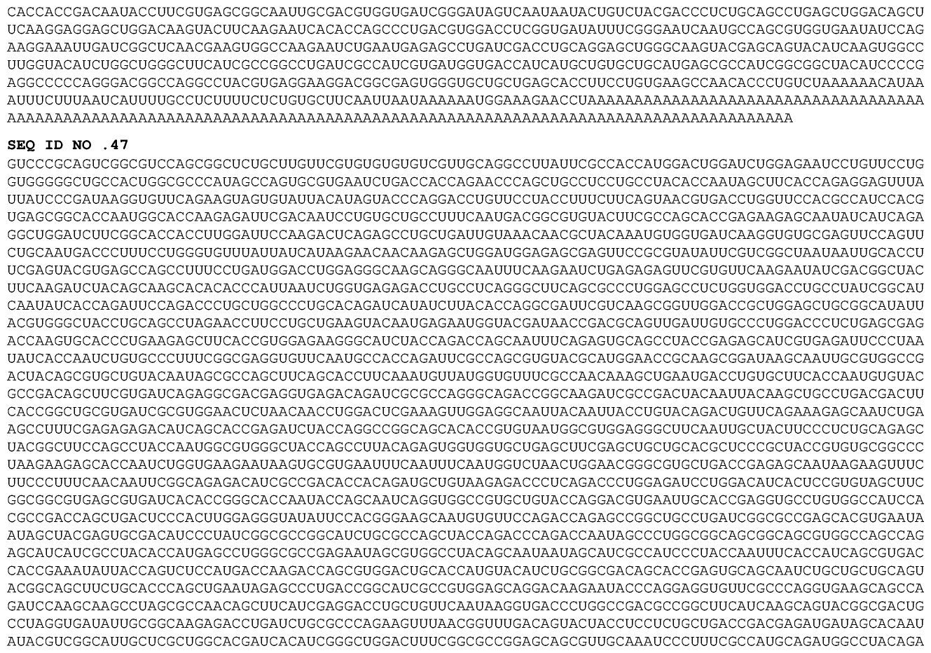

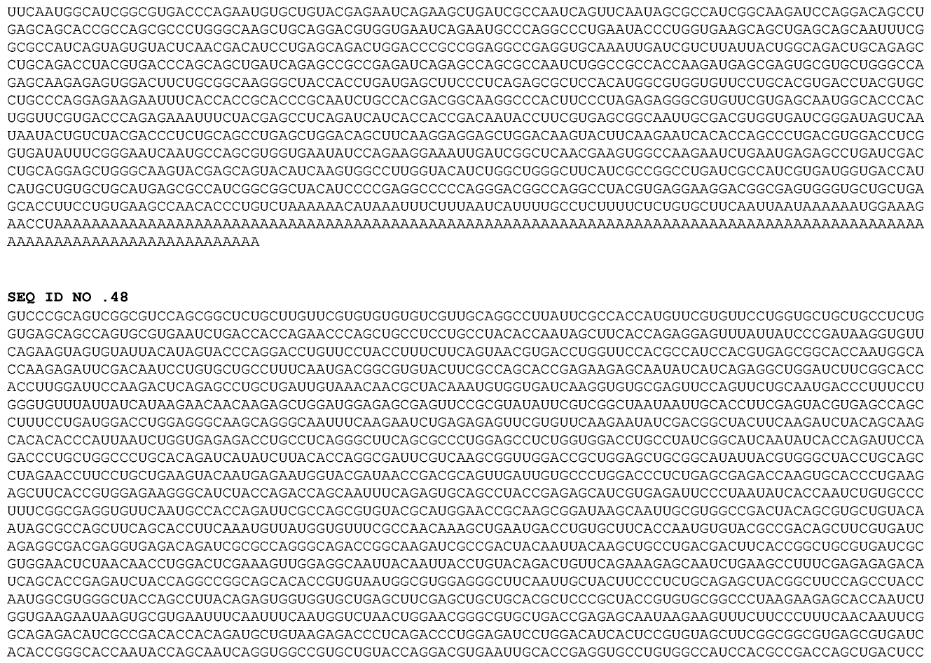

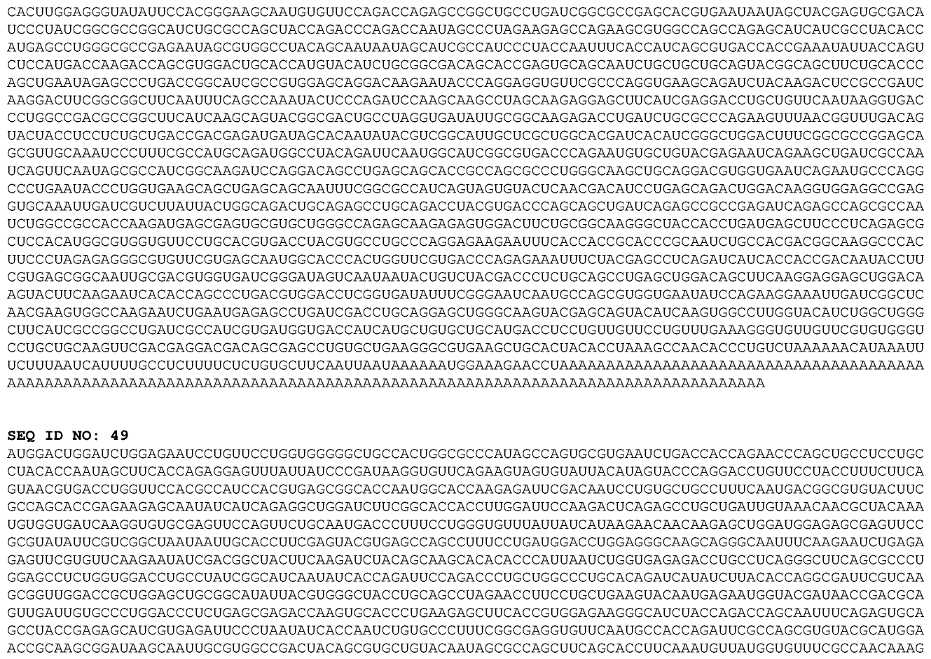

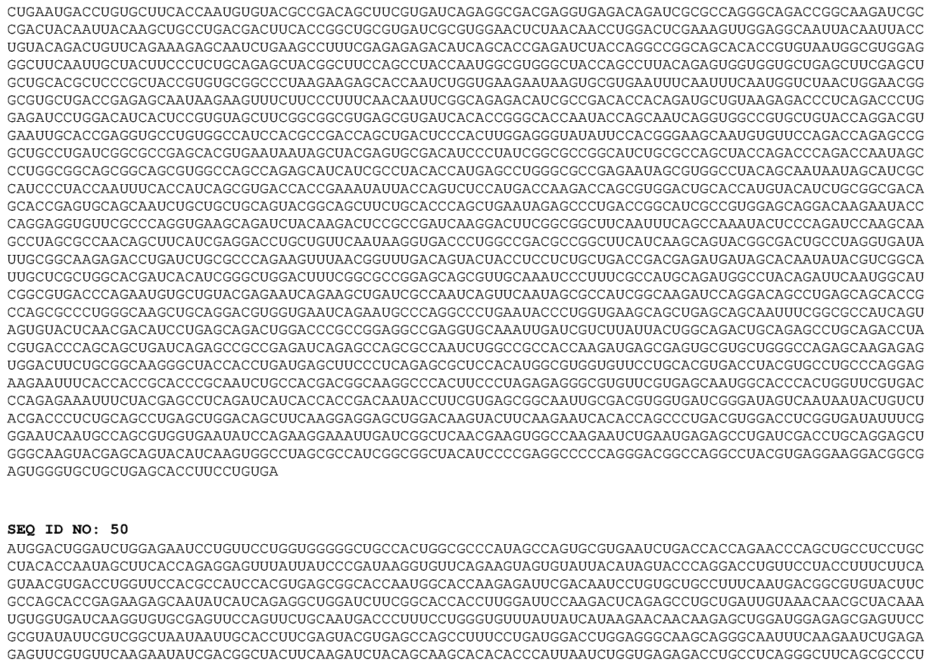

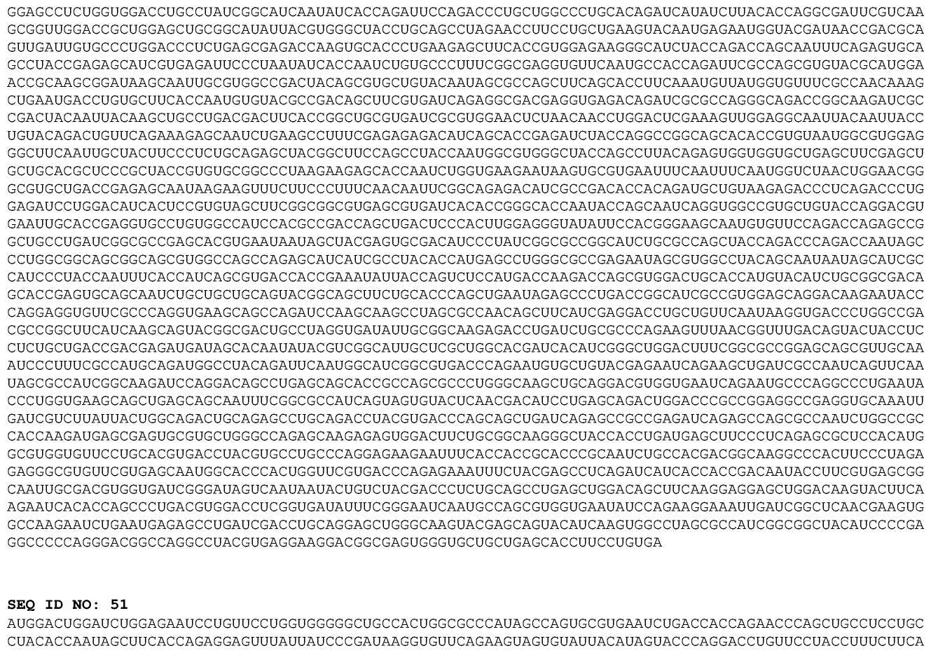

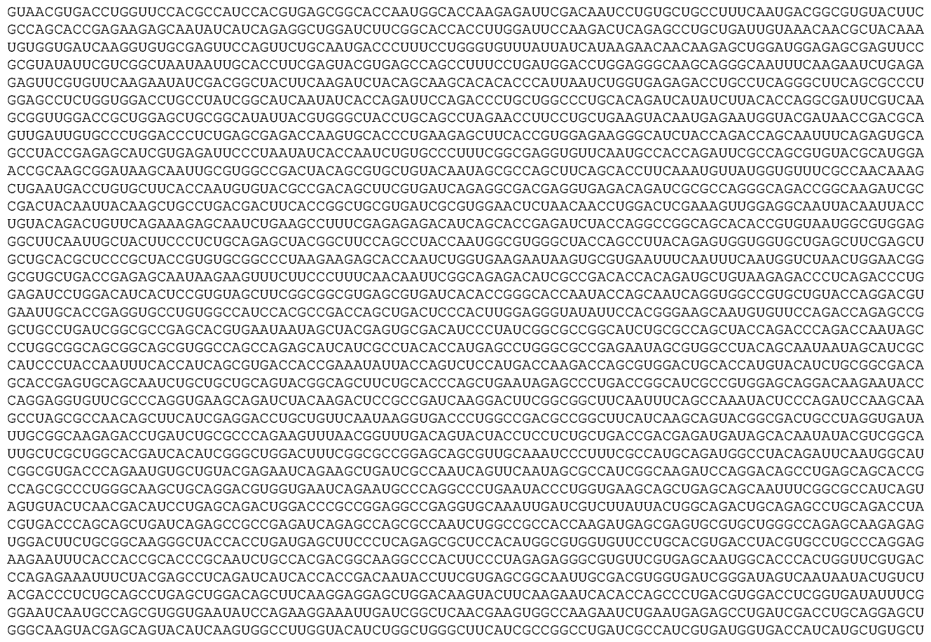

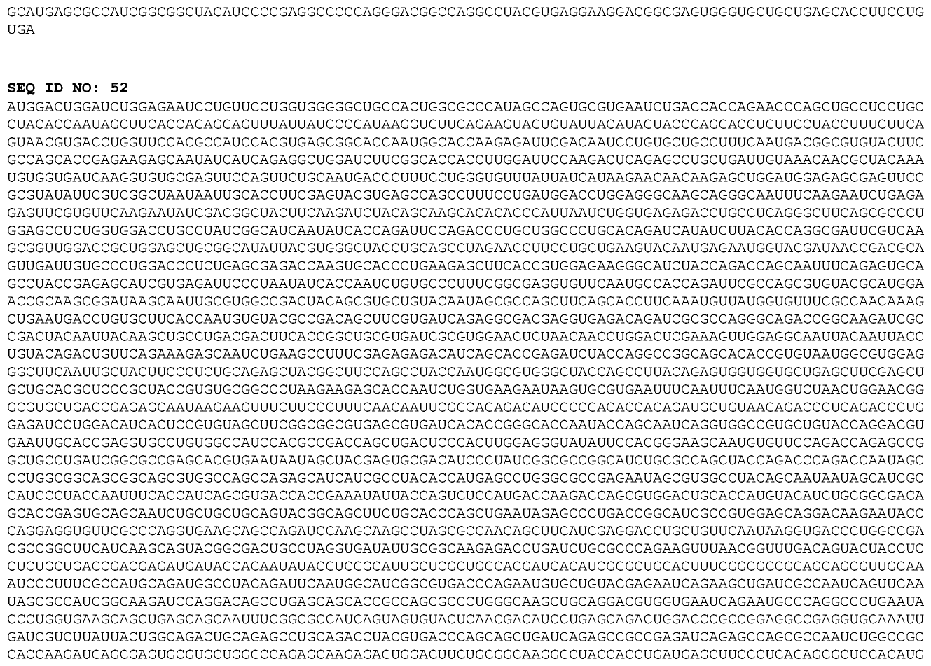

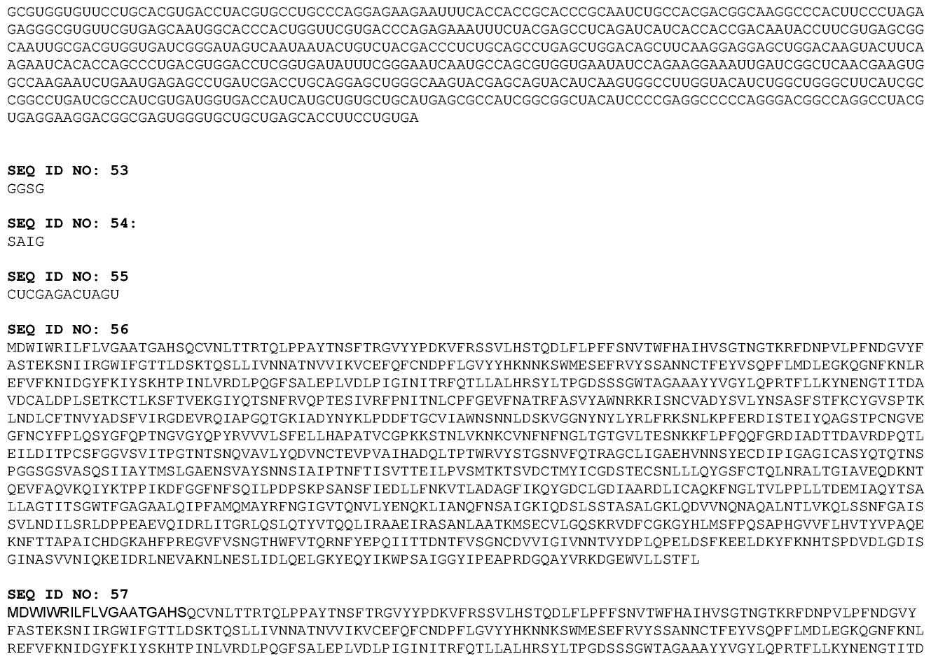

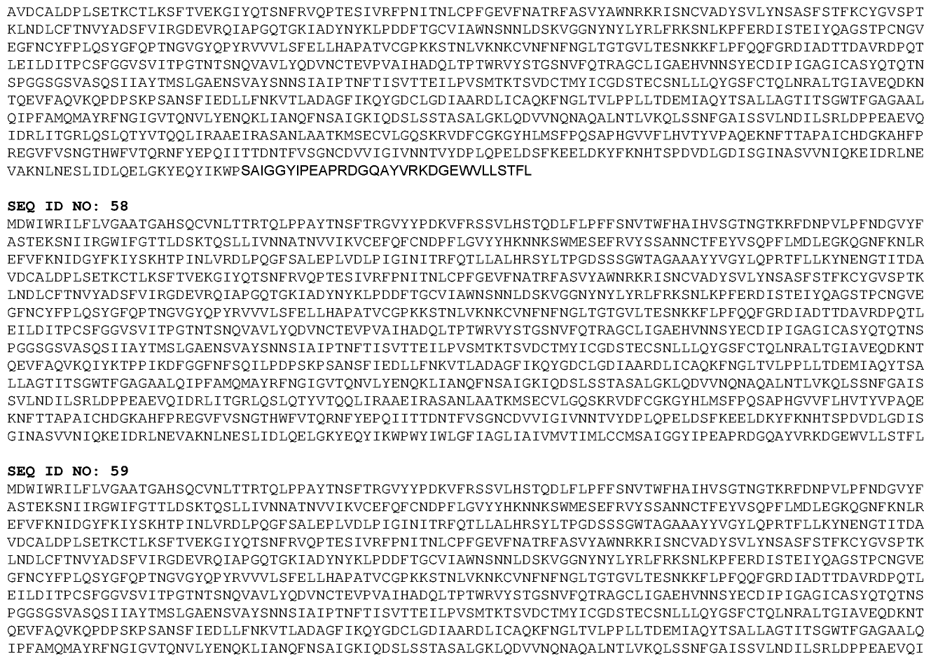

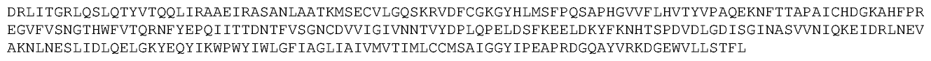

The results of the non-denaturing Western Blot are shown in FIG. 17. Specifically, the wild-type S protein showed a major band near 140 kDa (S protein monomer). By contrast, the expressed pre-fusion stable form of S protein trimer (S-Trimer) did not show any band near 140 kDa, but showed a major band at the top of the lane, indicating that the pre-fusion stable form of the S protein expressed by S protein mRNA formed a trimer.

In the present disclosure, superior effects were observed. For example, an efficient and safe SARS-CoV-2 mRNA vaccine was obtained, which was capable of expressing the SARS-CoV-2 virus S antigen protein in the pre-fusion stable form in animal, and triggering the animal's cellular and humoral immunity response, and inducing the production of specific antibodies in the animal.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A nucleic acid encoding a modified spike protein or fragment thereof derived from a coronavirus, wherein the modified spike protein or fragment thereof is locked in a pre-fusion state.

2. The nucleic acid of claim 1, wherein the coronavirus is SARS-CoV-2.

3. The nucleic acid of claim 1 or 2, wherein the modified spike protein or fragment thereof meets either one or both of the conditions:
(a) the amino acid that corresponds to K986 of SEQ ID NO: 1 is not Lys; and
(b) the amino acid that corresponds to V987 of SEQ ID NO: 1 is not Val.

4. The nucleic acid of claim 1 or 2, wherein the amino acid in the modified spike protein or fragment thereof that corresponds to K986 of SEQ ID NO: 1 is proline.

5. The nucleic acid of claim 1 or 2, wherein the amino acids in the modified spike protein or fragment thereof that corresponds to V987 of SEQ ID NO: 1 is proline.

6. The nucleic acid of claim 1 or 2, wherein both of the amino acids in the modified spike protein or fragment thereof that correspond to K986 and V987 of SEQ ID NO: 1 are proline.

7. The nucleic acid of any one of claims 1-6, wherein the modified spike protein or fragment thereof comprises from N-terminus to C-terminus: an N-terminal domain (NTD), a receptor binding domain (RBD), and a heptad repeat region; wherein the heptad repeat region comprises a first heptad repeat (HR1) and a second heptad repeat (HR2).

8. The nucleic acid of claim 7, wherein the N-terminal domain comprises a sequence that is at least 80% identical to amino acids 14-305 of SEQ ID NO: 1.

9. The nucleic acid of claim 7 or 8, wherein the receptor binding domain comprises a sequence that is at least 80% identical to amino acids 319-541 of SEQ ID NO: 1.

10. The nucleic acid of any one of claims 7-9, wherein the heptad repeat region comprises a sequence that is at least 80% identical to amino acids 912-1213 of SEQ ID NO: 1.

11. The nucleic acid of any one of claims 1-10, wherein the modified spike protein or fragment thereof further comprises a transmembrane domain (TM), wherein the transmembrane domain comprises a sequence that is at least 80% identical to amino acids 1214-1237 of SEQ ID NO: 1.

12. The nucleic acid of any one of claims 1-10, wherein the modified spike protein or fragment thereof further consists of an extracellular domain.

13. The nucleic acid of any one of claims 1-12, wherein the modified spike protein or fragment thereof does not have a membrane fusion peptide domain (e.g., the amino acids that correspond to positions 788-806 of SEQ ID NO: 1).

14. The nucleic acid of any one of claims 1-13, wherein the modified spike protein or fragment thereof is resistant to protease cleavage.

15. The nucleic acid of any one of claims 1-14, wherein the amino acids in the modified spike protein or fragment thereof that correspond to positions 682-685 of SEQ ID NO: 1 are GGS G.

16. The nucleic acid of any one of claims 1-15, wherein the amino acids in the modified spike protein or fragment thereof that correspond to positions 814 and 815 of SEQ ID NO: 1 are AN.

17. The nucleic acid of any one of claims 1-16, wherein the modified spike protein or fragment thereof further comprises a signal peptide.

18. The nucleic acid of claim 17, wherein the signal peptide comprises a sequence that is at least 80% identical to amino acids 1-13 of SEQ ID NO: 1.

19. The nucleic acid of claim 17, wherein the signal peptide is an immunoglobulin heavy chain variable region (IGVH) signal peptide, wherein the immunoglobulin heavy chain variable region (IGVH) signal peptide comprises a sequence that is at least 80% identical to SEQ ID NO: 39.

20. The nucleic acid of any one of claims 1-19, wherein the modified spike protein or fragment thereof further comprises a T4 phage fibritin trimer motif, wherein the T4 phage fibritin trimer motif comprises a sequence that is at least 80% identical to SEQ ID NO: 40.

21. The nucleic acid of claim 20, wherein the modified spike protein or fragment thereof further comprises a linker peptide sequence (e.g., SAIG (SEQ ID NO: 54)).

22. The nucleic acid of any one of claims 1-21, wherein the nucleic acid comprises a 5′-UTR, wherein the 5′-UTR is a Kozak sequence (SEQ ID NO. 42) or the 5′-UTR of the following genes: HBB (Hemoglobin Subunit Beta), Hsp70, DNAH2 (Dynein Axonemal Heavy Chain 2), or HSD17B4 (Hydroxysteroid 17-Beta Dehydrogenase 4).

23. The nucleic acid of any one of claims 1-22, wherein the modified spike protein or fragment thereof forms a trimer.

24. The nucleic acid of any one of claims 1-23, wherein the modified spike protein or fragment thereof comprises a sequence that is at least 80% identical to any one of SEQ ID NOs: 56-59; or SEQ ID NOs: 3-38, with or without amino acids 1-13 of SEQ ID NOs: 3-38.

25. The nucleic acid of any one of claims 1-24, wherein the modified spike protein or fragment thereof comprises a sequence that is at least 80% identical to amino acids 14-1213 of SEQ ID NO: 29.

26. The nucleic acid of any one of claims 1-24, wherein the modified spike protein or fragment thereof comprises a sequence that is at least 80% identical to amino acids 14-1194 of SEQ ID NO: 32.

27. The nucleic acid of any one of claims 1-24, wherein the modified spike protein or fragment thereof comprises a sequence that is at least 80% identical to amino acids 14-1237 of SEQ ID NO: 35.

28. The nucleic acid of any one of claims 1-24, wherein the modified spike protein or fragment thereof comprises a sequence that is at least 80% identical to amino acids 14-1218 of SEQ ID NO: 38.

29. The nucleic acid of any one of claims 25-28, wherein the modified spike protein or fragment thereof further comprises is an immunoglobulin heavy chain variable region (IGVH) signal peptide.

30. A nucleic acid encoding a modified spike protein or fragment thereof derived from a coronavirus, wherein the nucleic acid comprises a sequence that is at least 80% identical to any one of SEQ ID NOs: 2 and 44-52.

31. The nucleic acid of claim 30, wherein the nucleic acid comprises a sequence that is at least 80% identical to SEQ ID NO: 49.

32. The nucleic acid of claim 30, wherein the nucleic acid comprises a sequence that is at least 80% identical to SEQ ID NO: 50.

33. The nucleic acid of claim 30, wherein the nucleic acid comprises a sequence that is at least 80% identical to SEQ ID NO: 51.

34. The nucleic acid of claim 30, wherein the nucleic acid comprises a sequence that is at least 80% identical to SEQ ID NO: 52.

35. The nucleic acid of any one of claims 1-34, wherein the nucleic acid comprises from 5′ end to 3′ end the following elements:
a) 5′-UTR;
b) Kozak sequence;
c) an open reading frame encoding the modified spike protein or fragment thereof;
d) 3′-UTR; and
e) a polyA tail.

36. The nucleic acid of any one of claims 1-35, wherein the nucleic acid is a DNA molecule.

37. The nucleic acid of any one of claims 1-35, wherein the nucleic acid is an RNA molecule.

38. The nucleic acid of any one of claims 1-35, wherein the nucleic acid is a RNA molecule with one or more modified nucleosides.

39. The nucleic acid of claim 38, wherein the one or more modified nucleosides are selected from pseudo-UTP, 5-Me-CTP, rUTP, 1-N-Me-pseudo-UTP, or a combination thereof.

40. An expression vector comprising the nucleic acid of any one of claims 1-39 and a promoter, wherein the promoter is operably linked to the nucleic acid.

41. A modified coronavirus spike protein or fragment thereof, comprising from N-terminus to C-terminus: an N-terminal domain (NTD), a receptor binding domain (RBD), and a heptad repeat region; wherein the heptad repeat region comprises a first heptad repeat (HR1) and a second heptad repeat (HR2).

42. The modified coronavirus spike protein or fragment thereof of claim 41, wherein the modified spike protein or fragment thereof is locked in a pre-fusion state (e.g., a closed pre-fusion state).

43. The modified coronavirus spike protein or fragment thereof of claim 41 or 42, wherein the modified spike protein or fragment thereof meets either one or both of the conditions:
(a) the amino acid that corresponds to K986 of SEQ ID NO: 1 is not Lys (e.g., is Asp or Glu); and
(b) the amino acid that corresponds to V987 of SEQ ID NO: 1 is not V (e.g., is Phe, Try, or Trp).

44. The modified coronavirus spike protein or fragment thereof of any one of claims 41-43, wherein either one or both of the amino acids corresponding to K986 and V987 of SEQ ID NO: 1 are proline.

45. The modified coronavirus spike protein or fragment thereof of any one of claims 41-44, wherein the N-terminal domain comprises a sequence that is at least 80% identical to amino acids 14-305 of SEQ ID NO: 1.

46. The modified coronavirus spike protein or fragment thereof of any one of claims 41-45, wherein the receptor binding domain comprises a sequence that is at least 80% identical to amino acids 319-541 of SEQ ID NO: 1.

47. The modified coronavirus spike protein or fragment thereof of any one of claims 41-46, wherein the heptad repeat region comprises a sequence that is at least 80% identical to amino acids 912-1213 of SEQ ID NO: 1.

48. The modified coronavirus spike protein or fragment thereof of any one of claims 41-47, further comprising a transmembrane domain (TM), wherein the transmembrane domain comprises a sequence that is at least 80% identical to amino acids 1214-1237 of SEQ ID NO: 1.

49. The modified coronavirus spike protein or fragment thereof of any one of claims 41-48, wherein the modified coronavirus spike protein or fragment thereof does not have a membrane fusion peptide domain (e.g., the amino acids that correspond to positions 788-806 of SEQ ID NO: 1).

50. The modified coronavirus spike protein or fragment thereof of any one of claims 41-49, wherein the modified spike protein or fragment thereof is resistant to protease cleavage (e.g., Furin-like protease cleavage).

51. The modified coronavirus spike protein or fragment thereof of any one of claims 41-50, wherein the amino acids that correspond to positions 682-685 of SEQ ID NO: 1 are GGSG (SEQ ID NO: 53).

52. The modified coronavirus spike protein or fragment thereof of any one of claims 41-51, wherein the amino acids that correspond to positions 814 and 815 of SEQ ID NO: 1 are AN.

53. The modified coronavirus spike protein or fragment thereof of any one of claims 41-52, further comprising a signal peptide.

54. The modified coronavirus spike protein or fragment thereof of claim 53, wherein the signal peptide comprises a sequence that is at least 80% identical to amino acids 1-13 of SEQ ID NO: 1.

55. The modified coronavirus spike protein or fragment thereof of claim 53, wherein the signal peptide is an immunoglobulin heavy chain variable region (IGVH) signal peptide (e.g., a sequence that is at least 80% identical to SEQ ID NO: 39).

56. The modified coronavirus spike protein or fragment thereof of any one of claims 41-55, further comprising a linker peptide sequence and a T4 phage fibritin trimer motif.

57. The modified coronavirus spike protein or fragment thereof of claim 56, wherein the linker peptide sequence is SAIG (SEQ ID NO: 54).

58. The modified coronavirus spike protein or fragment thereof of any one of claims 41-57, wherein the T4 phage fibritin trimer motif comprises a sequence that is at least 80% identical to SEQ ID NO: 40.

59. A protein complex comprising three coronavirus spike proteins or fragments thereof, wherein each of the three coronavirus spike proteins or fragments comprises modified coronavirus spike protein or fragment thereof of any one of claims 41-58.

60. A vaccine comprising the nucleic acids of any one of claims 1-39, the modified coronavirus spike protein or fragment thereof of any one of claims 41-58, or the protein complex of claim 59.

61. A pharmaceutical composition comprising
the nucleic acids of any one of claims 1-39, the modified coronavirus spike protein or fragment thereof of any one of claims 41-58, or the protein complex of claim 59; and
a pharmaceutical carrier.

62. A lipid nanoparticle comprising the nucleic acids of any one of claims 1-39, the modified coronavirus spike protein or fragment thereof of any one of claims 41-58, or the protein complex of claim 59.

63. A method of inducing an immune response to coronavirus in a subject, wherein the method comprises administering to a subject in need thereof the nucleic acids of any one of claims 1-39, the modified coronavirus spike protein or fragment thereof of any one of claims 41-58, the protein complex of claim 59, the vaccine of claim 60, the pharmaceutical composition of claim 61, or the liposome nanoparticle of claim 62.

64. The method of claim 63, wherein the subject develops an immune response to coronavirus within 14 days of the administration.

65. The method of claim 63, wherein at least 2 doses are administered to the subject.

66. The methods of claim 65, wherein the second dose is administered at least 10 days (e.g., 14 days) after the first dose is administered to the subject.

67. The method of claim 65, wherein the subject maintains the immune response to coronavirus for at least 3 months.

68. The method of any one of claims 63-67, wherein the subject develops a neutralizing antibody against coronavirus in response.

69. A method of increasing an immune response to coronavirus in a subject or treating a subject having coronavirus, the method comprising: administering to the subject in need thereof the nucleic acids of any one of claims 1-39, the modified coronavirus spike protein or fragment thereof of any one of claims 41-58, the protein complex of claim 59, the vaccine of claim 60, the pharmaceutical composition of claim 61, or the liposome nanoparticle of claim 62.

70. The method of claim 69, wherein the subject is in coronavirus incubation period.

71. A method of making a nucleic acid vaccine comprising:
synthesizing the nucleic acid of any one of claims 1-39,

72. The method of claim 71, wherein the nucleic acid has been optimized for expression and/or translation efficiency.

73. The method of claim 71, wherein the nucleic acid is synthesized in the presence of pseudo-UTP.

74. The method of claim 71, wherein the nucleic acid is synthesized in the presence of 5-Me-CTP, and 1-N-Me-Pseudo-UTP.

75. The method of claim 71, wherein the nucleic acid is synthesized in a solution in the presence of rUTP and 1-N-Me-Pseudo-UTP, wherein the ratio of rUTP to 1-N-Me-Pseudo-UTP is between 1.5:1 and 1:1.5 (e.g., roughly about 1:1).

76. The method of claim 71, wherein the nucleic acid is synthesized in the presence of 1-N-Me-Pseudo-UTP.

77. The method of any one of claims 71-76, wherein the method further comprises packaging the nucleic acid in a liposomal nanoparticle.

78. A cell comprising the nucleic acids of any one of claims 1-39.

79. A cell expresses the modified coronavirus spike protein or fragment thereof of any one of claims 41-58.

80. A method of making an antibody the specifically binds to S protein, the method comprising immunizing an animal with the nucleic acids of any one of claims 1-39, the modified coronavirus spike protein or fragment thereof of any one of claims 41-58, the protein complex of claim 59, the vaccine of claim 60, the pharmaceutical composition of claim 61, and/or the liposome nanoparticle of claim 62.

81. The method of claim 80, wherein the animal is a non-human mammal (e.g., a mouse).

82. The method of claim 81, wherein the method further comprises humanizing the antibody.


SEQUENCE LISTING (66 sequences)

SEQ ID NO: 1 - protein, 1273 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 3822 bp, Artificial Sequence
  Description of Artificial Sequence Synthetic
atgttcgtgttcctggtgctgctgcctctggtgagcagccagtgcgtgaatctgaccaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatccacgtgagcggcaccaatggcaccaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctgggtgtttattatcataagaacaacaagagctggatggagagcgagttccgcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgccctggagcctctggtggacctgcctatcggcatcaatatcaccagattccagaccctgctggccctgcacagatcatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaacctggactcgaaagttggaggcaattacaattacctgtacagactgttcagaaagagcaatctgaagcctttcgagagagacatcagcaccgagatctaccaggccggcagcacaccgtgtaatggcgtggagggcttcaattgctacttccctctgcagagctacggcttccagcctaccaatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggcaccaataccagcaatcaggtggccgtgctgtaccaggacgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccctagaagagccagaagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcaagaggagcttcatcgaggacctgctgttcaataaggtgaccctggccgacgccggcttcatcaagcagtacggcgactgcctaggtgatattgcggcaagagacctgatctgcgcccagaagtttaacggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcaggacgtggtgaatcagaatgcccaggccctgaataccctggtgaagcagctgagcagcaatttcggcgccatcagtagtgtactcaacgacatcctgagcagactggacaaggtggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgcaagttcgacgaggacgacagcgagcctgtgctgaagggcgtgaagctgcactacacctaa

SEQ ID NO: 3 - protein, 1213 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 4 - protein, 1213 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 5 - protein, 1213 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 6 - protein, 1194 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 7 - protein, 1194 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 8 - protein, 1194 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 9 - protein, 1237 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCM

SEQ ID NO: 10 - protein, 1237 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCM

SEQ ID NO: 11 - protein, 1237 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCM

SEQ ID NO: 12 - protein, 1218 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCM

SEQ ID NO: 13 - protein, 1218 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCM

SEQ ID NO: 14 - protein, 1218 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCM

SEQ ID NO: 15 - protein, 1213 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 16 - protein, 1213 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 17 - protein, 1213 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 18 - protein, 1194 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 19 - protein, 1194 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 20 - protein, 1194 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 21 - protein, 1237 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCM

SEQ ID NO: 22 - protein, 1237 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCM

SEQ ID NO: 23 - protein, 1237 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCM

SEQ ID NO: 24 - protein, 1218 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCM

SEQ ID NO: 25 - protein, 1218 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCM

SEQ ID NO: 26 - protein, 1218 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCM

SEQ ID NO: 27 - protein, 1213 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 28 - protein, 1213 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 29 - protein, 1213 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 30 - protein, 1194 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 31 - protein, 1194 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 32 - protein, 1194 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 33 - protein, 1237 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCM

SEQ ID NO: 34 - protein, 1237 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCM

SEQ ID NO: 35 - protein, 1237 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCM

SEQ ID NO: 36 - protein, 1218 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCM

SEQ ID NO: 37 - protein, 1218 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCM

SEQ ID NO: 38 - protein, 1218 aa, Artificial Sequence
  Description of Artificial Sequence Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCM

SEQ ID NO: 39 - protein, 19 aa, Artificial Sequence
  Description of Artificial Sequence Synthetic
MDWIWRILFLVGAATGAHS

SEQ ID NO: 40 - protein, 27 aa, Artificial Sequence
  Description of Artificial Sequence Synthetic
GYIPEAPRDGQAYVRKDGEWVLLSTFL

SEQ ID NO: 41 - DNA, 62 bp, Artificial Sequence
  Description of Artificial Sequence Synthetic
gtcccgcagtcggcgtccagcggctctgcttgttcgtgtgtgtgtcgttgcaggccttattc

SEQ ID NO: 42 - DNA, 6 bp, Artificial Sequence
  Description of Artificial Sequence Synthetic
gccacc

SEQ ID NO: 43 - DNA, 89 bp, Artificial Sequence
  Description of Artificial Sequence Synthetic
agccaacaccctgtctaaaaaacataaatttctttaatcattttgcctcttttctctgtgcttcaattaataaaaaatggaaagaacct

SEQ ID NO: 44 - RNA, 4030 nt, Artificial Sequence
  Description of Artificial Sequence Synthetic
gucccgcagucggcguccagcggcucugcuuguucgugugugugucguugcaggccuuauucgccaccauggacuggaucuggagaauccuguuccuggugggggcugccacuggcgcccauagccagugcgugaaucugaccaccagaacccagcugccuccugccuacaccaauagcuucaccagaggaguuuauuaucccgauaagguguucagaaguaguguauuacauaguacccaggaccuguuccuaccuuucuucaguaacgugaccugguuccacgccauccacgugagcggcaccaauggcaccaagagauucgacaauccugugcugccuuucaaugacggcguguacuucgccagcaccgagaagagcaauaucaucagaggcuggaucuucggcaccaccuuggauuccaagacucagagccugcugauuguaaacaacgcuacaaauguggugaucaaggugugcgaguuccaguucugcaaugacccuuuccuggguguuuauuaucauaagaacaacaagagcuggauggagagcgaguuccgcguauauucgucggcuaauaauugcaccuucgaguacgugagccagccuuuccugauggaccuggagggcaagcagggcaauuucaagaaucugagagaguucguguucaagaauaucgacggcuacuucaagaucuacagcaagcacacacccauuaaucuggugagagaccugccucagggcuucagcgcccuggagccucugguggaccugccuaucggcaucaauaucaccagauuccagacccugcuggcccugcacagaucauaucuuacaccaggcgauucgucaagcgguuggaccgcuggagcugcggcauauuacgugggcuaccugcagccuagaaccuuccugcugaaguacaaugagaaugguacgauaaccgacgcaguugauugugcccuggacccucugagcgagaccaagugcacccugaagagcuucaccguggagaagggcaucuaccagaccagcaauuucagagugcagccuaccgagagcaucgugagauucccuaauaucaccaaucugugcccuuucggcgagguguucaaugccaccagauucgccagcguguacgcauggaaccgcaagcggauaagcaauugcguggccgacuacagcgugcuguacaauagcgccagcuucagcaccuucaaauguuaugguguuucgccaacaaagcugaaugaccugugcuucaccaauguguacgccgacagcuucgugaucagaggcgacgaggugagacagaucgcgccagggcagaccggcaagaucgccgacuacaauuacaagcugccugacgacuucaccggcugcgugaucgcguggaacucuaacaaccuggacucgaaaguuggaggcaauuacaauuaccuguacagacuguucagaaagagcaaucugaagccuuucgagagagacaucagcaccgagaucuaccaggccggcagcacaccguguaauggcguggagggcuucaauugcuacuucccucugcagagcuacggcuuccagccuaccaauggcgugggcuaccagccuuacagagugguggugcugagcuucgagcugcugcacgcucccgcuaccgugugcggcccuaagaagagcaccaaucuggugaagaauaagugcgugaauuucaauuucaauggucuaacuggaacgggcgugcugaccgagagcaauaagaaguuucuucccuuucaacaauucggcagagacaucgccgacaccacagaugcuguaagagacccucagacccuggagauccuggacaucacuccguguagcuucggcggcgugagcgugaucacaccgggcaccaauaccagcaaucagguggccgugcuguaccaggacgugaauugcaccgaggugccuguggccauccacgccgaccagcugacucccacuuggaggguauauuccacgggaagcaauguguuccagaccagagccggcugccugaucggcgccgagcacgugaauaauagcuacgagugcgacaucccuaucggcgccggcaucugcgccagcuaccagacccagaccaauagcccuggcggcagcggcagcguggccagccagagcaucaucgccuacaccaugagccugggcgccgagaauagcguggccuacagcaauaauagcaucgccaucccuaccaauuucaccaucagcgugaccaccgaaauauuaccagucuccaugaccaagaccagcguggacugcaccauguacaucugcggcgacagcaccgagugcagcaaucugcugcugcaguacggcagcuucugcacccagcugaauagagcccugaccggcaucgccguggagcaggacaagaauacccaggagguguucgcccaggugaagcagaucuacaagacuccgccgaucaaggacuucggcggcuucaauuucagccaaauacucccagauccaagcaagccuagcgccaacagcuucaucgaggaccugcuguucaauaaggugacccuggccgacgccggcuucaucaagcaguacggcgacugccuaggugauauugcggcaagagaccugaucugcgcccagaaguuuaacgguuugacaguacuaccuccucugcugaccgacgagaugauagcacaauauacgucggcauugcucgcuggcacgaucacaucgggcuggacuuucggcgccggagcagcguugcaaaucccuuucgccaugcagauggccuacagauucaauggcaucggcgugacccagaaugugcuguacgagaaucagaagcugaucgccaaucaguucaauagcgccaucggcaagauccaggacagccugagcagcaccgccagcgcccugggcaagcugcaggacguggugaaucagaaugcccaggcccugaauacccuggugaagcagcugagcagcaauuucggcgccaucaguaguguacucaacgacauccugagcagacuggacccgccggaggccgaggugcaaauugaucgucuuauuacuggcagacugcagagccugcagaccuacgugacccagcagcugaucagagccgccgagaucagagccagcgccaaucuggccgccaccaagaugagcgagugcgugcugggccagagcaagagaguggacuucugcggcaagggcuaccaccugaugagcuucccucagagcgcuccacauggcgugguguuccugcacgugaccuacgugccugcccaggagaagaauuucaccaccgcacccgcaaucugccacgacggcaaggcccacuucccuagagagggcguguucgugagcaauggcacccacugguucgugacccagagaaauuucuacgagccucagaucaucaccaccgacaauaccuucgugagcggcaauugcgacguggugaucgggauagucaauaauacugucuacgacccucugcagccugagcuggacagcuucaaggaggagcuggacaaguacuucaagaaucacaccagcccugacguggaccucggugauauuucgggaaucaaugccagcguggugaauauccagaaggaaauugaucggcucaacgaaguggccaagaaucugaaugagagccugaucgaccugcaggagcugggcaaguacgagcaguacaucaaguggccuagcgccaucggcggcuacauccccgaggcccccagggacggccaggccuacgugaggaaggacggcgagugggugcugcugagcaccuuccugugaagccaacacccugucuaaaaaacauaaauuucuuuaaucauuuugccucuuuucucugugcuucaauuaauaaaaaauggaaagaaccuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 45 - RNA, 3973 nt, Artificial Sequence
  Description of Artificial Sequence Synthetic
gucccgcagucggcguccagcggcucugcuuguucgugugugugucguugcaggccuuauucgccaccauggacuggaucuggagaauccuguuccuggugggggcugccacuggcgcccauagccagugcgugaaucugaccaccagaacccagcugccuccugccuacaccaauagcuucaccagaggaguuuauuaucccgauaagguguucagaaguaguguauuacauaguacccaggaccuguuccuaccuuucuucaguaacgugaccugguuccacgccauccacgugagcggcaccaauggcaccaagagauucgacaauccugugcugccuuucaaugacggcguguacuucgccagcaccgagaagagcaauaucaucagaggcuggaucuucggcaccaccuuggauuccaagacucagagccugcugauuguaaacaacgcuacaaauguggugaucaaggugugcgaguuccaguucugcaaugacccuuuccuggguguuuauuaucauaagaacaacaagagcuggauggagagcgaguuccgcguauauucgucggcuaauaauugcaccuucgaguacgugagccagccuuuccugauggaccuggagggcaagcagggcaauuucaagaaucugagagaguucguguucaagaauaucgacggcuacuucaagaucuacagcaagcacacacccauuaaucuggugagagaccugccucagggcuucagcgcccuggagccucugguggaccugccuaucggcaucaauaucaccagauuccagacccugcuggcccugcacagaucauaucuuacaccaggcgauucgucaagcgguuggaccgcuggagcugcggcauauuacgugggcuaccugcagccuagaaccuuccugcugaaguacaaugagaaugguacgauaaccgacgcaguugauugugcccuggacccucugagcgagaccaagugcacccugaagagcuucaccguggagaagggcaucuaccagaccagcaauuucagagugcagccuaccgagagcaucgugagauucccuaauaucaccaaucugugcccuuucggcgagguguucaaugccaccagauucgccagcguguacgcauggaaccgcaagcggauaagcaauugcguggccgacuacagcgugcuguacaauagcgccagcuucagcaccuucaaauguuaugguguuucgccaacaaagcugaaugaccugugcuucaccaauguguacgccgacagcuucgugaucagaggcgacgaggugagacagaucgcgccagggcagaccggcaagaucgccgacuacaauuacaagcugccugacgacuucaccggcugcgugaucgcguggaacucuaacaaccuggacucgaaaguuggaggcaauuacaauuaccuguacagacuguucagaaagagcaaucugaagccuuucgagagagacaucagcaccgagaucuaccaggccggcagcacaccguguaauggcguggagggcuucaauugcuacuucccucugcagagcuacggcuuccagccuaccaauggcgugggcuaccagccuuacagagugguggugcugagcuucgagcugcugcacgcucccgcuaccgugugcggcccuaagaagagcaccaaucuggugaagaauaagugcgugaauuucaauuucaauggucuaacuggaacgggcgugcugaccgagagcaauaagaaguuucuucccuuucaacaauucggcagagacaucgccgacaccacagaugcuguaagagacccucagacccuggagauccuggacaucacuccguguagcuucggcggcgugagcgugaucacaccgggcaccaauaccagcaaucagguggccgugcuguaccaggacgugaauugcaccgaggugccuguggccauccacgccgaccagcugacucccacuuggaggguauauuccacgggaagcaauguguuccagaccagagccggcugccugaucggcgccgagcacgugaauaauagcuacgagugcgacaucccuaucggcgccggcaucugcgccagcuaccagacccagaccaauagcccuggcggcagcggcagcguggccagccagagcaucaucgccuacaccaugagccugggcgccgagaauagcguggccuacagcaauaauagcaucgccaucccuaccaauuucaccaucagcgugaccaccgaaauauuaccagucuccaugaccaagaccagcguggacugcaccauguacaucugcggcgacagcaccgagugcagcaaucugcugcugcaguacggcagcuucugcacccagcugaauagagcccugaccggcaucgccguggagcaggacaagaauacccaggagguguucgcccaggugaagcagccagauccaagcaagccuagcgccaacagcuucaucgaggaccugcuguucaauaaggugacccuggccgacgccggcuucaucaagcaguacggcgacugccuaggugauauugcggcaagagaccugaucugcgcccagaaguuuaacgguuugacaguacuaccuccucugcugaccgacgagaugauagcacaauauacgucggcauugcucgcuggcacgaucacaucgggcuggacuuucggcgccggagcagcguugcaaaucccuuucgccaugcagauggccuacagauucaauggcaucggcgugacccagaaugugcuguacgagaaucagaagcugaucgccaaucaguucaauagcgccaucggcaagauccaggacagccugagcagcaccgccagcgcccugggcaagcugcaggacguggugaaucagaaugcccaggcccugaauacccuggugaagcagcugagcagcaauuucggcgccaucaguaguguacucaacgacauccugagcagacuggacccgccggaggccgaggugcaaauugaucgucuuauuacuggcagacugcagagccugcagaccuacgugacccagcagcugaucagagccgccgagaucagagccagcgccaaucuggccgccaccaagaugagcgagugcgugcugggccagagcaagagaguggacuucugcggcaagggcuaccaccugaugagcuucccucagagcgcuccacauggcgugguguuccugcacgugaccuacgugccugcccaggagaagaauuucaccaccgcacccgcaaucugccacgacggcaaggcccacuucccuagagagggcguguucgugagcaauggcacccacugguucgugacccagagaaauuucuacgagccucagaucaucaccaccgacaauaccuucgugagcggcaauugcgacguggugaucgggauagucaauaauacugucuacgacccucugcagccugagcuggacagcuucaaggaggagcuggacaaguacuucaagaaucacaccagcccugacguggaccucggugauauuucgggaaucaaugccagcguggugaauauccagaaggaaauugaucggcucaacgaaguggccaagaaucugaaugagagccugaucgaccugcaggagcugggcaaguacgagcaguacaucaaguggccuagcgccaucggcggcuacauccccgaggcccccagggacggccaggccuacgugaggaaggacggcgagugggugcugcugagcaccuuccugugaagccaacacccugucuaaaaaacauaaauuucuuuaaucauuuugccucuuuucucugugcuucaauuaauaaaaaauggaaagaaccuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 46 - RNA, 4102 nt, Artificial Sequence
  Description of Artificial Sequence Synthetic
gucccgcagucggcguccagcggcucugcuuguucgugugugugucguugcaggccuuauucgccaccauggacuggaucuggagaauccuguuccuggugggggcugccacuggcgcccauagccagugcgugaaucugaccaccagaacccagcugccuccugccuacaccaauagcuucaccagaggaguuuauuaucccgauaagguguucagaaguaguguauuacauaguacccaggaccuguuccuaccuuucuucaguaacgugaccugguuccacgccauccacgugagcggcaccaauggcaccaagagauucgacaauccugugcugccuuucaaugacggcguguacuucgccagcaccgagaagagcaauaucaucagaggcuggaucuucggcaccaccuuggauuccaagacucagagccugcugauuguaaacaacgcuacaaauguggugaucaaggugugcgaguuccaguucugcaaugacccuuuccuggguguuuauuaucauaagaacaacaagagcuggauggagagcgaguuccgcguauauucgucggcuaauaauugcaccuucgaguacgugagccagccuuuccugauggaccuggagggcaagcagggcaauuucaagaaucugagagaguucguguucaagaauaucgacggcuacuucaagaucuacagcaagcacacacccauuaaucuggugagagaccugccucagggcuucagcgcccuggagccucugguggaccugccuaucggcaucaauaucaccagauuccagacccugcuggcccugcacagaucauaucuuacaccaggcgauucgucaagcgguuggaccgcuggagcugcggcauauuacgugggcuaccugcagccuagaaccuuccugcugaaguacaaugagaaugguacgauaaccgacgcaguugauugugcccuggacccucugagcgagaccaagugcacccugaagagcuucaccguggagaagggcaucuaccagaccagcaauuucagagugcagccuaccgagagcaucgugagauucccuaauaucaccaaucugugcccuuucggcgagguguucaaugccaccagauucgccagcguguacgcauggaaccgcaagcggauaagcaauugcguggccgacuacagcgugcuguacaauagcgccagcuucagcaccuucaaauguuaugguguuucgccaacaaagcugaaugaccugugcuucaccaauguguacgccgacagcuucgugaucagaggcgacgaggugagacagaucgcgccagggcagaccggcaagaucgccgacuacaauuacaagcugccugacgacuucaccggcugcgugaucgcguggaacucuaacaaccuggacucgaaaguuggaggcaauuacaauuaccuguacagacuguucagaaagagcaaucugaagccuuucgagagagacaucagcaccgagaucuaccaggccggcagcacaccguguaauggcguggagggcuucaauugcuacuucccucugcagagcuacggcuuccagccuaccaauggcgugggcuaccagccuuacagagugguggugcugagcuucgagcugcugcacgcucccgcuaccgugugcggcccuaagaagagcaccaaucuggugaagaauaagugcgugaauuucaauuucaauggucuaacuggaacgggcgugcugaccgagagcaauaagaaguuucuucccuuucaacaauucggcagagacaucgccgacaccacagaugcuguaagagacccucagacccuggagauccuggacaucacuccguguagcuucggcggcgugagcgugaucacaccgggcaccaauaccagcaaucagguggccgugcuguaccaggacgugaauugcaccgaggugccuguggccauccacgccgaccagcugacucccacuuggaggguauauuccacgggaagcaauguguuccagaccagagccggcugccugaucggcgccgagcacgugaauaauagcuacgagugcgacaucccuaucggcgccggcaucugcgccagcuaccagacccagaccaauagcccuggcggcagcggcagcguggccagccagagcaucaucgccuacaccaugagccugggcgccgagaauagcguggccuacagcaauaauagcaucgccaucccuaccaauuucaccaucagcgugaccaccgaaauauuaccagucuccaugaccaagaccagcguggacugcaccauguacaucugcggcgacagcaccgagugcagcaaucugcugcugcaguacggcagcuucugcacccagcugaauagagcccugaccggcaucgccguggagcaggacaagaauacccaggagguguucgcccaggugaagcagaucuacaagacuccgccgaucaaggacuucggcggcuucaauuucagccaaauacucccagauccaagcaagccuagcgccaacagcuucaucgaggaccugcuguucaauaaggugacccuggccgacgccggcuucaucaagcaguacggcgacugccuaggugauauugcggcaagagaccugaucugcgcccagaaguuuaacgguuugacaguacuaccuccucugcugaccgacgagaugauagcacaauauacgucggcauugcucgcuggcacgaucacaucgggcuggacuuucggcgccggagcagcguugcaaaucccuuucgccaugcagauggccuacagauucaauggcaucggcgugacccagaaugugcuguacgagaaucagaagcugaucgccaaucaguucaauagcgccaucggcaagauccaggacagccugagcagcaccgccagcgcccugggcaagcugcaggacguggugaaucagaaugcccaggcccugaauacccuggugaagcagcugagcagcaauuucggcgccaucaguaguguacucaacgacauccugagcagacuggacccgccggaggccgaggugcaaauugaucgucuuauuacuggcagacugcagagccugcagaccuacgugacccagcagcugaucagagccgccgagaucagagccagcgccaaucuggccgccaccaagaugagcgagugcgugcugggccagagcaagagaguggacuucugcggcaagggcuaccaccugaugagcuucccucagagcgcuccacauggcgugguguuccugcacgugaccuacgugccugcccaggagaagaauuucaccaccgcacccgcaaucugccacgacggcaaggcccacuucccuagagagggcguguucgugagcaauggcacccacugguucgugacccagagaaauuucuacgagccucagaucaucaccaccgacaauaccuucgugagcggcaauugcgacguggugaucgggauagucaauaauacugucuacgacccucugcagccugagcuggacagcuucaaggaggagcuggacaaguacuucaagaaucacaccagcccugacguggaccucggugauauuucgggaaucaaugccagcguggugaauauccagaaggaaauugaucggcucaacgaaguggccaagaaucugaaugagagccugaucgaccugcaggagcugggcaaguacgagcaguacaucaaguggccuugguacaucuggcugggcuucaucgccggccugaucgccaucgugauggugaccaucaugcugugcugcaugagcgccaucggcggcuacauccccgaggcccccagggacggccaggccuacgugaggaaggacggcgagugggugcugcugagcaccuuccugugaagccaacacccugucuaaaaaacauaaauuucuuuaaucauuuugccucuuuucucugugcuucaauuaauaaaaaauggaaagaaccuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 47 - RNA, 4045 nt, Artificial Sequence
  Description of Artificial Sequence Synthetic
gucccgcagucggcguccagcggcucugcuuguucgugugugugucguugcaggccuuauucgccaccauggacuggaucuggagaauccuguuccuggugggggcugccacuggcgcccauagccagugcgugaaucugaccaccagaacccagcugccuccugccuacaccaauagcuucaccagaggaguuuauuaucccgauaagguguucagaaguaguguauuacauaguacccaggaccuguuccuaccuuucuucaguaacgugaccugguuccacgccauccacgugagcggcaccaauggcaccaagagauucgacaauccugugcugccuuucaaugacggcguguacuucgccagcaccgagaagagcaauaucaucagaggcuggaucuucggcaccaccuuggauuccaagacucagagccugcugauuguaaacaacgcuacaaauguggugaucaaggugugcgaguuccaguucugcaaugacccuuuccuggguguuuauuaucauaagaacaacaagagcuggauggagagcgaguuccgcguauauucgucggcuaauaauugcaccuucgaguacgugagccagccuuuccugauggaccuggagggcaagcagggcaauuucaagaaucugagagaguucguguucaagaauaucgacggcuacuucaagaucuacagcaagcacacacccauuaaucuggugagagaccugccucagggcuucagcgcccuggagccucugguggaccugccuaucggcaucaauaucaccagauuccagacccugcuggcccugcacagaucauaucuuacaccaggcgauucgucaagcgguuggaccgcuggagcugcggcauauuacgugggcuaccugcagccuagaaccuuccugcugaaguacaaugagaaugguacgauaaccgacgcaguugauugugcccuggacccucugagcgagaccaagugcacccugaagagcuucaccguggagaagggcaucuaccagaccagcaauuucagagugcagccuaccgagagcaucgugagauucccuaauaucaccaaucugugcccuuucggcgagguguucaaugccaccagauucgccagcguguacgcauggaaccgcaagcggauaagcaauugcguggccgacuacagcgugcuguacaauagcgccagcuucagcaccuucaaauguuaugguguuucgccaacaaagcugaaugaccugugcuucaccaauguguacgccgacagcuucgugaucagaggcgacgaggugagacagaucgcgccagggcagaccggcaagaucgccgacuacaauuacaagcugccugacgacuucaccggcugcgugaucgcguggaacucuaacaaccuggacucgaaaguuggaggcaauuacaauuaccuguacagacuguucagaaagagcaaucugaagccuuucgagagagacaucagcaccgagaucuaccaggccggcagcacaccguguaauggcguggagggcuucaauugcuacuucccucugcagagcuacggcuuccagccuaccaauggcgugggcuaccagccuuacagagugguggugcugagcuucgagcugcugcacgcucccgcuaccgugugcggcccuaagaagagcaccaaucuggugaagaauaagugcgugaauuucaauuucaauggucuaacuggaacgggcgugcugaccgagagcaauaagaaguuucuucccuuucaacaauucggcagagacaucgccgacaccacagaugcuguaagagacccucagacccuggagauccuggacaucacuccguguagcuucggcggcgugagcgugaucacaccgggcaccaauaccagcaaucagguggccgugcuguaccaggacgugaauugcaccgaggugccuguggccauccacgccgaccagcugacucccacuuggaggguauauuccacgggaagcaauguguuccagaccagagccggcugccugaucggcgccgagcacgugaauaauagcuacgagugcgacaucccuaucggcgccggcaucugcgccagcuaccagacccagaccaauagcccuggcggcagcggcagcguggccagccagagcaucaucgccuacaccaugagccugggcgccgagaauagcguggccuacagcaauaauagcaucgccaucccuaccaauuucaccaucagcgugaccaccgaaauauuaccagucuccaugaccaagaccagcguggacugcaccauguacaucugcggcgacagcaccgagugcagcaaucugcugcugcaguacggcagcuucugcacccagcugaauagagcccugaccggcaucgccguggagcaggacaagaauacccaggagguguucgcccaggugaagcagccagauccaagcaagccuagcgccaacagcuucaucgaggaccugcuguucaauaaggugacccuggccgacgccggcuucaucaagcaguacggcgacugccuaggugauauugcggcaagagaccugaucugcgcccagaaguuuaacgguuugacaguacuaccuccucugcugaccgacgagaugauagcacaauauacgucggcauugcucgcuggcacgaucacaucgggcuggacuuucggcgccggagcagcguugcaaaucccuuucgccaugcagauggccuacagauucaauggcaucggcgugacccagaaugugcuguacgagaaucagaagcugaucgccaaucaguucaauagcgccaucggcaagauccaggacagccugagcagcaccgccagcgcccugggcaagcugcaggacguggugaaucagaaugcccaggcccugaauacccuggugaagcagcugagcagcaauuucggcgccaucaguaguguacucaacgacauccugagcagacuggacccgccggaggccgaggugcaaauugaucgucuuauuacuggcagacugcagagccugcagaccuacgugacccagcagcugaucagagccgccgagaucagagccagcgccaaucuggccgccaccaagaugagcgagugcgugcugggccagagcaagagaguggacuucugcggcaagggcuaccaccugaugagcuucccucagagcgcuccacauggcgugguguuccugcacgugaccuacgugccugcccaggagaagaauuucaccaccgcacccgcaaucugccacgacggcaaggcccacuucccuagagagggcguguucgugagcaauggcacccacugguucgugacccagagaaauuucuacgagccucagaucaucaccaccgacaauaccuucgugagcggcaauugcgacguggugaucgggauagucaauaauacugucuacgacccucugcagccugagcuggacagcuucaaggaggagcuggacaaguacuucaagaaucacaccagcccugacguggaccucggugauauuucgggaaucaaugccagcguggugaauauccagaaggaaauugaucggcucaacgaaguggccaagaaucugaaugagagccugaucgaccugcaggagcugggcaaguacgagcaguacaucaaguggccuugguacaucuggcugggcuucaucgccggccugaucgccaucgugauggugaccaucaugcugugcugcaugagcgccaucggcggcuacauccccgaggcccccagggacggccaggccuacgugaggaaggacggcgagugggugcugcugagcaccuuccugugaagccaacacccugucuaaaaaacauaaauuucuuuaaucauuuugccucuuuucucugugcuucaauuaauaaaaaauggaaagaaccuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 48 - RNA, 4099 nt, Artificial Sequence
  Description of Artificial Sequence Synthetic
gucccgcagucggcguccagcggcucugcuuguucgugugugugucguugcaggccuuauucgccaccauguucguguuccuggugcugcugccucuggugagcagccagugcgugaaucugaccaccagaacccagcugccuccugccuacaccaauagcuucaccagaggaguuuauuaucccgauaagguguucagaaguaguguauuacauaguacccaggaccuguuccuaccuuucuucaguaacgugaccugguuccacgccauccacgugagcggcaccaauggcaccaagagauucgacaauccugugcugccuuucaaugacggcguguacuucgccagcaccgagaagagcaauaucaucagaggcuggaucuucggcaccaccuuggauuccaagacucagagccugcugauuguaaacaacgcuacaaauguggugaucaaggugugcgaguuccaguucugcaaugacccuuuccuggguguuuauuaucauaagaacaacaagagcuggauggagagcgaguuccgcguauauucgucggcuaauaauugcaccuucgaguacgugagccagccuuuccugauggaccuggagggcaagcagggcaauuucaagaaucugagagaguucguguucaagaauaucgacggcuacuucaagaucuacagcaagcacacacccauuaaucuggugagagaccugccucagggcuucagcgcccuggagccucugguggaccugccuaucggcaucaauaucaccagauuccagacccugcuggcccugcacagaucauaucuuacaccaggcgauucgucaagcgguuggaccgcuggagcugcggcauauuacgugggcuaccugcagccuagaaccuuccugcugaaguacaaugagaaugguacgauaaccgacgcaguugauugugcccuggacccucugagcgagaccaagugcacccugaagagcuucaccguggagaagggcaucuaccagaccagcaauuucagagugcagccuaccgagagcaucgugagauucccuaauaucaccaaucugugcccuuucggcgagguguucaaugccaccagauucgccagcguguacgcauggaaccgcaagcggauaagcaauugcguggccgacuacagcgugcuguacaauagcgccagcuucagcaccuucaaauguuaugguguuucgccaacaaagcugaaugaccugugcuucaccaauguguacgccgacagcuucgugaucagaggcgacgaggugagacagaucgcgccagggcagaccggcaagaucgccgacuacaauuacaagcugccugacgacuucaccggcugcgugaucgcguggaacucuaacaaccuggacucgaaaguuggaggcaauuacaauuaccuguacagacuguucagaaagagcaaucugaagccuuucgagagagacaucagcaccgagaucuaccaggccggcagcacaccguguaauggcguggagggcuucaauugcuacuucccucugcagagcuacggcuuccagccuaccaauggcgugggcuaccagccuuacagagugguggugcugagcuucgagcugcugcacgcucccgcuaccgugugcggcccuaagaagagcaccaaucuggugaagaauaagugcgugaauuucaauuucaauggucuaacuggaacgggcgugcugaccgagagcaauaagaaguuucuucccuuucaacaauucggcagagacaucgccgacaccacagaugcuguaagagacccucagacccuggagauccuggacaucacuccguguagcuucggcggcgugagcgugaucacaccgggcaccaauaccagcaaucagguggccgugcuguaccaggacgugaauugcaccgaggugccuguggccauccacgccgaccagcugacucccacuuggaggguauauuccacgggaagcaauguguuccagaccagagccggcugccugaucggcgccgagcacgugaauaauagcuacgagugcgacaucccuaucggcgccggcaucugcgccagcuaccagacccagaccaauagcccuagaagagccagaagcguggccagccagagcaucaucgccuacaccaugagccugggcgccgagaauagcguggccuacagcaauaauagcaucgccaucccuaccaauuucaccaucagcgugaccaccgaaauauuaccagucuccaugaccaagaccagcguggacugcaccauguacaucugcggcgacagcaccgagugcagcaaucugcugcugcaguacggcagcuucugcacccagcugaauagagcccugaccggcaucgccguggagcaggacaagaauacccaggagguguucgcccaggugaagcagaucuacaagacuccgccgaucaaggacuucggcggcuucaauuucagccaaauacucccagauccaagcaagccuagcaagaggagcuucaucgaggaccugcuguucaauaaggugacccuggccgacgccggcuucaucaagcaguacggcgacugccuaggugauauugcggcaagagaccugaucugcgcccagaaguuuaacgguuugacaguacuaccuccucugcugaccgacgagaugauagcacaauauacgucggcauugcucgcuggcacgaucacaucgggcuggacuuucggcgccggagcagcguugcaaaucccuuucgccaugcagauggccuacagauucaauggcaucggcgugacccagaaugugcuguacgagaaucagaagcugaucgccaaucaguucaauagcgccaucggcaagauccaggacagccugagcagcaccgccagcgcccugggcaagcugcaggacguggugaaucagaaugcccaggcccugaauacccuggugaagcagcugagcagcaauuucggcgccaucaguaguguacucaacgacauccugagcagacuggacaagguggaggccgaggugcaaauugaucgucuuauuacuggcagacugcagagccugcagaccuacgugacccagcagcugaucagagccgccgagaucagagccagcgccaaucuggccgccaccaagaugagcgagugcgugcugggccagagcaagagaguggacuucugcggcaagggcuaccaccugaugagcuucccucagagcgcuccacauggcgugguguuccugcacgugaccuacgugccugcccaggagaagaauuucaccaccgcacccgcaaucugccacgacggcaaggcccacuucccuagagagggcguguucgugagcaauggcacccacugguucgugacccagagaaauuucuacgagccucagaucaucaccaccgacaauaccuucgugagcggcaauugcgacguggugaucgggauagucaauaauacugucuacgacccucugcagccugagcuggacagcuucaaggaggagcuggacaaguacuucaagaaucacaccagcccugacguggaccucggugauauuucgggaaucaaugccagcguggugaauauccagaaggaaauugaucggcucaacgaaguggccaagaaucugaaugagagccugaucgaccugcaggagcugggcaaguacgagcaguacaucaaguggccuugguacaucuggcugggcuucaucgccggccugaucgccaucgugauggugaccaucaugcugugcugcaugaccuccuguuguuccuguuugaaaggguguuguucguguggguccugcugcaaguucgacgaggacgacagcgagccugugcugaagggcgugaagcugcacuacaccuaaagccaacacccugucuaaaaaacauaaauuucuuuaaucauuuugccucuuuucucugugcuucaauuaauaaaaaauggaaagaaccuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 49 - RNA, 3753 nt, Artificial Sequence
  Description of Artificial Sequence Synthetic
auggacuggaucuggagaauccuguuccuggugggggcugccacuggcgcccauagccagugcgugaaucugaccaccagaacccagcugccuccugccuacaccaauagcuucaccagaggaguuuauuaucccgauaagguguucagaaguaguguauuacauaguacccaggaccuguuccuaccuuucuucaguaacgugaccugguuccacgccauccacgugagcggcaccaauggcaccaagagauucgacaauccugugcugccuuucaaugacggcguguacuucgccagcaccgagaagagcaauaucaucagaggcuggaucuucggcaccaccuuggauuccaagacucagagccugcugauuguaaacaacgcuacaaauguggugaucaaggugugcgaguuccaguucugcaaugacccuuuccuggguguuuauuaucauaagaacaacaagagcuggauggagagcgaguuccgcguauauucgucggcuaauaauugcaccuucgaguacgugagccagccuuuccugauggaccuggagggcaagcagggcaauuucaagaaucugagagaguucguguucaagaauaucgacggcuacuucaagaucuacagcaagcacacacccauuaaucuggugagagaccugccucagggcuucagcgcccuggagccucugguggaccugccuaucggcaucaauaucaccagauuccagacccugcuggcccugcacagaucauaucuuacaccaggcgauucgucaagcgguuggaccgcuggagcugcggcauauuacgugggcuaccugcagccuagaaccuuccugcugaaguacaaugagaaugguacgauaaccgacgcaguugauugugcccuggacccucugagcgagaccaagugcacccugaagagcuucaccguggagaagggcaucuaccagaccagcaauuucagagugcagccuaccgagagcaucgugagauucccuaauaucaccaaucugugcccuuucggcgagguguucaaugccaccagauucgccagcguguacgcauggaaccgcaagcggauaagcaauugcguggccgacuacagcgugcuguacaauagcgccagcuucagcaccuucaaauguuaugguguuucgccaacaaagcugaaugaccugugcuucaccaauguguacgccgacagcuucgugaucagaggcgacgaggugagacagaucgcgccagggcagaccggcaagaucgccgacuacaauuacaagcugccugacgacuucaccggcugcgugaucgcguggaacucuaacaaccuggacucgaaaguuggaggcaauuacaauuaccuguacagacuguucagaaagagcaaucugaagccuuucgagagagacaucagcaccgagaucuaccaggccggcagcacaccguguaauggcguggagggcuucaauugcuacuucccucugcagagcuacggcuuccagccuaccaauggcgugggcuaccagccuuacagagugguggugcugagcuucgagcugcugcacgcucccgcuaccgugugcggcccuaagaagagcaccaaucuggugaagaauaagugcgugaauuucaauuucaauggucuaacuggaacgggcgugcugaccgagagcaauaagaaguuucuucccuuucaacaauucggcagagacaucgccgacaccacagaugcuguaagagacccucagacccuggagauccuggacaucacuccguguagcuucggcggcgugagcgugaucacaccgggcaccaauaccagcaaucagguggccgugcuguaccaggacgugaauugcaccgaggugccuguggccauccacgccgaccagcugacucccacuuggaggguauauuccacgggaagcaauguguuccagaccagagccggcugccugaucggcgccgagcacgugaauaauagcuacgagugcgacaucccuaucggcgccggcaucugcgccagcuaccagacccagaccaauagcccuggcggcagcggcagcguggccagccagagcaucaucgccuacaccaugagccugggcgccgagaauagcguggccuacagcaauaauagcaucgccaucccuaccaauuucaccaucagcgugaccaccgaaauauuaccagucuccaugaccaagaccagcguggacugcaccauguacaucugcggcgacagcaccgagugcagcaaucugcugcugcaguacggcagcuucugcacccagcugaauagagcccugaccggcaucgccguggagcaggacaagaauacccaggagguguucgcccaggugaagcagaucuacaagacuccgccgaucaaggacuucggcggcuucaauuucagccaaauacucccagauccaagcaagccuagcgccaacagcuucaucgaggaccugcuguucaauaaggugacccuggccgacgccggcuucaucaagcaguacggcgacugccuaggugauauugcggcaagagaccugaucugcgcccagaaguuuaacgguuugacaguacuaccuccucugcugaccgacgagaugauagcacaauauacgucggcauugcucgcuggcacgaucacaucgggcuggacuuucggcgccggagcagcguugcaaaucccuuucgccaugcagauggccuacagauucaauggcaucggcgugacccagaaugugcuguacgagaaucagaagcugaucgccaaucaguucaauagcgccaucggcaagauccaggacagccugagcagcaccgccagcgcccugggcaagcugcaggacguggugaaucagaaugcccaggcccugaauacccuggugaagcagcugagcagcaauuucggcgccaucaguaguguacucaacgacauccugagcagacuggacccgccggaggccgaggugcaaauugaucgucuuauuacuggcagacugcagagccugcagaccuacgugacccagcagcugaucagagccgccgagaucagagccagcgccaaucuggccgccaccaagaugagcgagugcgugcugggccagagcaagagaguggacuucugcggcaagggcuaccaccugaugagcuucccucagagcgcuccacauggcgugguguuccugcacgugaccuacgugccugcccaggagaagaauuucaccaccgcacccgcaaucugccacgacggcaaggcccacuucccuagagagggcguguucgugagcaauggcacccacugguucgugacccagagaaauuucuacgagccucagaucaucaccaccgacaauaccuucgugagcggcaauugcgacguggugaucgggauagucaauaauacugucuacgacccucugcagccugagcuggacagcuucaaggaggagcuggacaaguacuucaagaaucacaccagcccugacguggaccucggugauauuucgggaaucaaugccagcguggugaauauccagaaggaaauugaucggcucaacgaaguggccaagaaucugaaugagagccugaucgaccugcaggagcugggcaaguacgagcaguacaucaaguggccuagcgccaucggcggcuacauccccgaggcccccagggacggccaggccuacgugaggaaggacggcgagugggugcugcugagcaccuuccuguga

SEQ ID NO: 50 - RNA, 3696 nt, Artificial Sequence
  Description of Artificial Sequence Synthetic
auggacuggaucuggagaauccuguuccuggugggggcugccacuggcgcccauagccagugcgugaaucugaccaccagaacccagcugccuccugccuacaccaauagcuucaccagaggaguuuauuaucccgauaagguguucagaaguaguguauuacauaguacccaggaccuguuccuaccuuucuucaguaacgugaccugguuccacgccauccacgugagcggcaccaauggcaccaagagauucgacaauccugugcugccuuucaaugacggcguguacuucgccagcaccgagaagagcaauaucaucagaggcuggaucuucggcaccaccuuggauuccaagacucagagccugcugauuguaaacaacgcuacaaauguggugaucaaggugugcgaguuccaguucugcaaugacccuuuccuggguguuuauuaucauaagaacaacaagagcuggauggagagcgaguuccgcguauauucgucggcuaauaauugcaccuucgaguacgugagccagccuuuccugauggaccuggagggcaagcagggcaauuucaagaaucugagagaguucguguucaagaauaucgacggcuacuucaagaucuacagcaagcacacacccauuaaucuggugagagaccugccucagggcuucagcgcccuggagccucugguggaccugccuaucggcaucaauaucaccagauuccagacccugcuggcccugcacagaucauaucuuacaccaggcgauucgucaagcgguuggaccgcuggagcugcggcauauuacgugggcuaccugcagccuagaaccuuccugcugaaguacaaugagaaugguacgauaaccgacgcaguugauugugcccuggacccucugagcgagaccaagugcacccugaagagcuucaccguggagaagggcaucuaccagaccagcaauuucagagugcagccuaccgagagcaucgugagauucccuaauaucaccaaucugugcccuuucggcgagguguucaaugccaccagauucgccagcguguacgcauggaaccgcaagcggauaagcaauugcguggccgacuacagcgugcuguacaauagcgccagcuucagcaccuucaaauguuaugguguuucgccaacaaagcugaaugaccugugcuucaccaauguguacgccgacagcuucgugaucagaggcgacgaggugagacagaucgcgccagggcagaccggcaagaucgccgacuacaauuacaagcugccugacgacuucaccggcugcgugaucgcguggaacucuaacaaccuggacucgaaaguuggaggcaauuacaauuaccuguacagacuguucagaaagagcaaucugaagccuuucgagagagacaucagcaccgagaucuaccaggccggcagcacaccguguaauggcguggagggcuucaauugcuacuucccucugcagagcuacggcuuccagccuaccaauggcgugggcuaccagccuuacagagugguggugcugagcuucgagcugcugcacgcucccgcuaccgugugcggcccuaagaagagcaccaaucuggugaagaauaagugcgugaauuucaauuucaauggucuaacuggaacgggcgugcugaccgagagcaauaagaaguuucuucccuuucaacaauucggcagagacaucgccgacaccacagaugcuguaagagacccucagacccuggagauccuggacaucacuccguguagcuucggcggcgugagcgugaucacaccgggcaccaauaccagcaaucagguggccgugcuguaccaggacgugaauugcaccgaggugccuguggccauccacgccgaccagcugacucccacuuggaggguauauuccacgggaagcaauguguuccagaccagagccggcugccugaucggcgccgagcacgugaauaauagcuacgagugcgacaucccuaucggcgccggcaucugcgccagcuaccagacccagaccaauagcccuggcggcagcggcagcguggccagccagagcaucaucgccuacaccaugagccugggcgccgagaauagcguggccuacagcaauaauagcaucgccaucccuaccaauuucaccaucagcgugaccaccgaaauauuaccagucuccaugaccaagaccagcguggacugcaccauguacaucugcggcgacagcaccgagugcagcaaucugcugcugcaguacggcagcuucugcacccagcugaauagagcccugaccggcaucgccguggagcaggacaagaauacccaggagguguucgcccaggugaagcagccagauccaagcaagccuagcgccaacagcuucaucgaggaccugcuguucaauaaggugacccuggccgacgccggcuucaucaagcaguacggcgacugccuaggugauauugcggcaagagaccugaucugcgcccagaaguuuaacgguuugacaguacuaccuccucugcugaccgacgagaugauagcacaauauacgucggcauugcucgcuggcacgaucacaucgggcuggacuuucggcgccggagcagcguugcaaaucccuuucgccaugcagauggccuacagauucaauggcaucggcgugacccagaaugugcuguacgagaaucagaagcugaucgccaaucaguucaauagcgccaucggcaagauccaggacagccugagcagcaccgccagcgcccugggcaagcugcaggacguggugaaucagaaugcccaggcccugaauacccuggugaagcagcugagcagcaauuucggcgccaucaguaguguacucaacgacauccugagcagacuggacccgccggaggccgaggugcaaauugaucgucuuauuacuggcagacugcagagccugcagaccuacgugacccagcagcugaucagagccgccgagaucagagccagcgccaaucuggccgccaccaagaugagcgagugcgugcugggccagagcaagagaguggacuucugcggcaagggcuaccaccugaugagcuucccucagagcgcuccacauggcgugguguuccugcacgugaccuacgugccugcccaggagaagaauuucaccaccgcacccgcaaucugccacgacggcaaggcccacuucccuagagagggcguguucgugagcaauggcacccacugguucgugacccagagaaauuucuacgagccucagaucaucaccaccgacaauaccuucgugagcggcaauugcgacguggugaucgggauagucaauaauacugucuacgacccucugcagccugagcuggacagcuucaaggaggagcuggacaaguacuucaagaaucacaccagcccugacguggaccucggugauauuucgggaaucaaugccagcguggugaauauccagaaggaaauugaucggcucaacgaaguggccaagaaucugaaugagagccugaucgaccugcaggagcugggcaaguacgagcaguacaucaaguggccuagcgccaucggcggcuacauccccgaggcccccagggacggccaggccuacgugaggaaggacggcgagugggugcugcugagcaccuuccuguga

SEQ ID NO: 51 - RNA, 3825 nt, Artificial Sequence
  Description of Artificial Sequence Synthetic
auggacuggaucuggagaauccuguuccuggugggggcugccacuggcgcccauagccagugcgugaaucugaccaccagaacccagcugccuccugccuacaccaauagcuucaccagaggaguuuauuaucccgauaagguguucagaaguaguguauuacauaguacccaggaccuguuccuaccuuucuucaguaacgugaccugguuccacgccauccacgugagcggcaccaauggcaccaagagauucgacaauccugugcugccuuucaaugacggcguguacuucgccagcaccgagaagagcaauaucaucagaggcuggaucuucggcaccaccuuggauuccaagacucagagccugcugauuguaaacaacgcuacaaauguggugaucaaggugugcgaguuccaguucugcaaugacccuuuccuggguguuuauuaucauaagaacaacaagagcuggauggagagcgaguuccgcguauauucgucggcuaauaauugcaccuucgaguacgugagccagccuuuccugauggaccuggagggcaagcagggcaauuucaagaaucugagagaguucguguucaagaauaucgacggcuacuucaagaucuacagcaagcacacacccauuaaucuggugagagaccugccucagggcuucagcgcccuggagccucugguggaccugccuaucggcaucaauaucaccagauuccagacccugcuggcccugcacagaucauaucuuacaccaggcgauucgucaagcgguuggaccgcuggagcugcggcauauuacgugggcuaccugcagccuagaaccuuccugcugaaguacaaugagaaugguacgauaaccgacgcaguugauugugcccuggacccucugagcgagaccaagugcacccugaagagcuucaccguggagaagggcaucuaccagaccagcaauuucagagugcagccuaccgagagcaucgugagauucccuaauaucaccaaucugugcccuuucggcgagguguucaaugccaccagauucgccagcguguacgcauggaaccgcaagcggauaagcaauugcguggccgacuacagcgugcuguacaauagcgccagcuucagcaccuucaaauguuaugguguuucgccaacaaagcugaaugaccugugcuucaccaauguguacgccgacagcuucgugaucagaggcgacgaggugagacagaucgcgccagggcagaccggcaagaucgccgacuacaauuacaagcugccugacgacuucaccggcugcgugaucgcguggaacucuaacaaccuggacucgaaaguuggaggcaauuacaauuaccuguacagacuguucagaaagagcaaucugaagccuuucgagagagacaucagcaccgagaucuaccaggccggcagcacaccguguaauggcguggagggcuucaauugcuacuucccucugcagagcuacggcuuccagccuaccaauggcgugggcuaccagccuuacagagugguggugcugagcuucgagcugcugcacgcucccgcuaccgugugcggcccuaagaagagcaccaaucuggugaagaauaagugcgugaauuucaauuucaauggucuaacuggaacgggcgugcugaccgagagcaauaagaaguuucuucccuuucaacaauucggcagagacaucgccgacaccacagaugcuguaagagacccucagacccuggagauccuggacaucacuccguguagcuucggcggcgugagcgugaucacaccgggcaccaauaccagcaaucagguggccgugcuguaccaggacgugaauugcaccgaggugccuguggccauccacgccgaccagcugacucccacuuggaggguauauuccacgggaagcaauguguuccagaccagagccggcugccugaucggcgccgagcacgugaauaauagcuacgagugcgacaucccuaucggcgccggcaucugcgccagcuaccagacccagaccaauagcccuggcggcagcggcagcguggccagccagagcaucaucgccuacaccaugagccugggcgccgagaauagcguggccuacagcaauaauagcaucgccaucccuaccaauuucaccaucagcgugaccaccgaaauauuaccagucuccaugaccaagaccagcguggacugcaccauguacaucugcggcgacagcaccgagugcagcaaucugcugcugcaguacggcagcuucugcacccagcugaauagagcccugaccggcaucgccguggagcaggacaagaauacccaggagguguucgcccaggugaagcagaucuacaagacuccgccgaucaaggacuucggcggcuucaauuucagccaaauacucccagauccaagcaagccuagcgccaacagcuucaucgaggaccugcuguucaauaaggugacccuggccgacgccggcuucaucaagcaguacggcgacugccuaggugauauugcggcaagagaccugaucugcgcccagaaguuuaacgguuugacaguacuaccuccucugcugaccgacgagaugauagcacaauauacgucggcauugcucgcuggcacgaucacaucgggcuggacuuucggcgccggagcagcguugcaaaucccuuucgccaugcagauggccuacagauucaauggcaucggcgugacccagaaugugcuguacgagaaucagaagcugaucgccaaucaguucaauagcgccaucggcaagauccaggacagccugagcagcaccgccagcgcccugggcaagcugcaggacguggugaaucagaaugcccaggcccugaauacccuggugaagcagcugagcagcaauuucggcgccaucaguaguguacucaacgacauccugagcagacuggacccgccggaggccgaggugcaaauugaucgucuuauuacuggcagacugcagagccugcagaccuacgugacccagcagcugaucagagccgccgagaucagagccagcgccaaucuggccgccaccaagaugagcgagugcgugcugggccagagcaagagaguggacuucugcggcaagggcuaccaccugaugagcuucccucagagcgcuccacauggcgugguguuccugcacgugaccuacgugccugcccaggagaagaauuucaccaccgcacccgcaaucugccacgacggcaaggcccacuucccuagagagggcguguucgugagcaauggcacccacugguucgugacccagagaaauuucuacgagccucagaucaucaccaccgacaauaccuucgugagcggcaauugcgacguggugaucgggauagucaauaauacugucuacgacccucugcagccugagcuggacagcuucaaggaggagcuggacaaguacuucaagaaucacaccagcccugacguggaccucggugauauuucgggaaucaaugccagcguggugaauauccagaaggaaauugaucggcucaacgaaguggccaagaaucugaaugagagccugaucgaccugcaggagcugggcaaguacgagcaguacaucaaguggccuugguacaucuggcugggcuucaucgccggccugaucgccaucgugauggugaccaucaugcugugcugcaugagcgccaucggcggcuacauccccgaggcccccagggacggccaggccuacgugaggaaggacggcgagugggugcugcugagcaccuuccuguga

SEQ ID NO: 52 - RNA, 3768 nt, Artificial Sequence
  Description of Artificial Sequence Synthetic
auggacuggaucuggagaauccuguuccuggugggggcugccacuggcgcccauagccagugcgugaaucugaccaccagaacccagcugccuccugccuacaccaauagcuucaccagaggaguuuauuaucccgauaagguguucagaaguaguguauuacauaguacccaggaccuguuccuaccuuucuucaguaacgugaccugguuccacgccauccacgugagcggcaccaauggcaccaagagauucgacaauccugugcugccuuucaaugacggcguguacuucgccagcaccgagaagagcaauaucaucagaggcuggaucuucggcaccaccuuggauuccaagacucagagccugcugauuguaaacaacgcuacaaauguggugaucaaggugugcgaguuccaguucugcaaugacccuuuccuggguguuuauuaucauaagaacaacaagagcuggauggagagcgaguuccgcguauauucgucggcuaauaauugcaccuucgaguacgugagccagccuuuccugauggaccuggagggcaagcagggcaauuucaagaaucugagagaguucguguucaagaauaucgacggcuacuucaagaucuacagcaagcacacacccauuaaucuggugagagaccugccucagggcuucagcgcccuggagccucugguggaccugccuaucggcaucaauaucaccagauuccagacccugcuggcccugcacagaucauaucuuacaccaggcgauucgucaagcgguuggaccgcuggagcugcggcauauuacgugggcuaccugcagccuagaaccuuccugcugaaguacaaugagaaugguacgauaaccgacgcaguugauugugcccuggacccucugagcgagaccaagugcacccugaagagcuucaccguggagaagggcaucuaccagaccagcaauuucagagugcagccuaccgagagcaucgugagauucccuaauaucaccaaucugugcccuuucggcgagguguucaaugccaccagauucgccagcguguacgcauggaaccgcaagcggauaagcaauugcguggccgacuacagcgugcuguacaauagcgccagcuucagcaccuucaaauguuaugguguuucgccaacaaagcugaaugaccugugcuucaccaauguguacgccgacagcuucgugaucagaggcgacgaggugagacagaucgcgccagggcagaccggcaagaucgccgacuacaauuacaagcugccugacgacuucaccggcugcgugaucgcguggaacucuaacaaccuggacucgaaaguuggaggcaauuacaauuaccuguacagacuguucagaaagagcaaucugaagccuuucgagagagacaucagcaccgagaucuaccaggccggcagcacaccguguaauggcguggagggcuucaauugcuacuucccucugcagagcuacggcuuccagccuaccaauggcgugggcuaccagccuuacagagugguggugcugagcuucgagcugcugcacgcucccgcuaccgugugcggcccuaagaagagcaccaaucuggugaagaauaagugcgugaauuucaauuucaauggucuaacuggaacgggcgugcugaccgagagcaauaagaaguuucuucccuuucaacaauucggcagagacaucgccgacaccacagaugcuguaagagacccucagacccuggagauccuggacaucacuccguguagcuucggcggcgugagcgugaucacaccgggcaccaauaccagcaaucagguggccgugcuguaccaggacgugaauugcaccgaggugccuguggccauccacgccgaccagcugacucccacuuggaggguauauuccacgggaagcaauguguuccagaccagagccggcugccugaucggcgccgagcacgugaauaauagcuacgagugcgacaucccuaucggcgccggcaucugcgccagcuaccagacccagaccaauagcccuggcggcagcggcagcguggccagccagagcaucaucgccuacaccaugagccugggcgccgagaauagcguggccuacagcaauaauagcaucgccaucccuaccaauuucaccaucagcgugaccaccgaaauauuaccagucuccaugaccaagaccagcguggacugcaccauguacaucugcggcgacagcaccgagugcagcaaucugcugcugcaguacggcagcuucugcacccagcugaauagagcccugaccggcaucgccguggagcaggacaagaauacccaggagguguucgcccaggugaagcagccagauccaagcaagccuagcgccaacagcuucaucgaggaccugcuguucaauaaggugacccuggccgacgccggcuucaucaagcaguacggcgacugccuaggugauauugcggcaagagaccugaucugcgcccagaaguuuaacgguuugacaguacuaccuccucugcugaccgacgagaugauagcacaauauacgucggcauugcucgcuggcacgaucacaucgggcuggacuuucggcgccggagcagcguugcaaaucccuuucgccaugcagauggccuacagauucaauggcaucggcgugacccagaaugugcuguacgagaaucagaagcugaucgccaaucaguucaauagcgccaucggcaagauccaggacagccugagcagcaccgccagcgcccugggcaagcugcaggacguggugaaucagaaugcccaggcccugaauacccuggugaagcagcugagcagcaauuucggcgccaucaguaguguacucaacgacauccugagcagacuggacccgccggaggccgaggugcaaauugaucgucuuauuacuggcagacugcagagccugcagaccuacgugacccagcagcugaucagagccgccgagaucagagccagcgccaaucuggccgccaccaagaugagcgagugcgugcugggccagagcaagagaguggacuucugcggcaagggcuaccaccugaugagcuucccucagagcgcuccacauggcgugguguuccugcacgugaccuacgugccugcccaggagaagaauuucaccaccgcacccgcaaucugccacgacggcaaggcccacuucccuagagagggcguguucgugagcaauggcacccacugguucgugacccagagaaauuucuacgagccucagaucaucaccaccgacaauaccuucgugagcggcaauugcgacguggugaucgggauagucaauaauacugucuacgacccucugcagccugagcuggacagcuucaaggaggagcuggacaaguacuucaagaaucacaccagcccugacguggaccucggugauauuucgggaaucaaugccagcguggugaauauccagaaggaaauugaucggcucaacgaaguggccaagaaucugaaugagagccugaucgaccugcaggagcugggcaaguacgagcaguacaucaaguggccuugguacaucuggcugggcuucaucgccggccugaucgccaucgugauggugaccaucaugcugugcugcaugagcgccaucggcggcuacauccccgaggcccccagggacggccaggccuacgugaggaaggacggcgagugggugcugcugagcaccuuccuguga

SEQ ID NO: 53 - protein, 4 aa, Artificial Sequence
  Description of Artificial Sequence Synthetic
GGSG

SEQ ID NO: 54 - protein, 4 aa, Artificial Sequence
  Description of Artificial Sequence Synthetic
SAIG

SEQ ID NO: 55 - RNA, 12 nt, Artificial Sequence
  Description of Artificial Sequence Synthetic
cucgagacuagu

SEQ ID NO: 56 - protein, 1250 aa, Artificial Sequence
  Description of Artificial Sequence Synthetic
MDWIWRILFLVGAATGAHSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSAIGGYIPEAPRDGQAYVRKDGEWVLLSTFL

SEQ ID NO: 57 - protein, 1231 aa, Artificial Sequence
  Description of Artificial Sequence Synthetic
MDWIWRILFLVGAATGAHSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSAIGGYIPEAPRDGQAYVRKDGEWVLLSTFL

SEQ ID NO: 58 - protein, 1274 aa, Artificial Sequence
  Description of Artificial Sequence Synthetic
MDWIWRILFLVGAATGAHSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMSAIGGYIPEAPRDGQAYVRKDGEWVLLSTFL

SEQ ID NO: 59 - protein, 1255 aa, Artificial Sequence
  Description of Artificial Sequence Synthetic
MDWIWRILFLVGAATGAHSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMSAIGGYIPEAPRDGQAYVRKDGEWVLLSTFL

SEQ ID NO: 60 - DNA, 20 bp, Artificial Sequence
  Description of Artificial Sequence Synthetic
taatacgactcactataggg

SEQ ID NO: 61 - DNA, 20 bp, Artificial Sequence
  Description of Artificial Sequence Synthetic
taatacgactcactatacgg

SEQ ID NO: 62 - DNA, 20 bp, Artificial Sequence
  Description of Artificial Sequence Synthetic
taatacgactcactataagg

SEQ ID NO: 63 - DNA, 20 bp, Artificial Sequence
  Description of Artificial Sequence Synthetic
attatgctgagtgatatccc

SEQ ID NO: 64 - DNA, 20 bp, Artificial Sequence
  Description of Artificial Sequence Synthetic
attatgctgagtgatatgcc

SEQ ID NO: 65 - DNA, 20 bp, Artificial Sequence
  Description of Artificial Sequence Synthetic
attatgctgagtgatattcc

SEQ ID NO: 66 - DNA, 17 bp, Artificial Sequence
  Description of Artificial Sequence Synthetic
taatacgactcactata